(19) United States
(12) Patent Application Publication
(10) Pub. No.: US 2026/0055385 A1
(43) Pub. Date: Feb. 26, 2026

(54) EVOLVED INTEGRASES AND METHODS OF USING THE SAME FOR GENOME EDITING

(71) Applicant: University of Hawaii, Honolulu, HI (US)

(72) Inventors: Jesse B. OWENS, Honolulu, HI (US); Brian HEW, Honolulu, HI (US)

(21) Appl. No.: 19/250,849

(22) Filed: Jun. 26, 2025

Related U.S. Application Data
(63) Continuation of application No. 18/915,656, filed on Oct. 15, 2024, now Pat. No. 12,378,539.
Application No. 18/915,656 is a continuation of application No. PCT/US2024/035414, filed on Jun. 25, 2024.
(60) Provisional application No. 63/510,171, filed on Jun. 26, 2023.
(60) Provisional application No. 63/592,945, filed on Oct. 25, 2023.

(51) Int. Cl. C12N 9/22

(52) U.S. Cl. CPC C12N 9/22

(57) ABSTRACT

Disclosed are evolved Bxb1 and PhiC31 integrases that exhibit increased insertion activity and efficiency as compared to their wild-type counterparts and use of the evolved integrases to modify any sequence within the genome of a cell or subject.

This application claims benefit from U.S. Provisional Patent Application Serial Nos. 63/510,171, filed Jun. 26, 2023, and 63/592,945, filed Oct. 25, 2023, the contents of which are incorporated herein by reference in their entireties.

### INTRODUCTION

This invention was made with government support under grant nos. GM103457, GM132779, GM125526 and EB031124 awarded by the National Institutes of Health. The government has certain rights in this invention.

### STATEMENT REGARDING ELECTRONIC FILING OF A SEQUENCE LISTING

A Sequence Listing in XML format, entitled “UH0010WO_ST26.XML,” 342,786 bytes in size, generated Jun. 3, 2024, and filed herewith, is hereby incorporated by reference into the specification.

### BACKGROUND

Many genetic diseases arise from disparate mutations differing from one patient to the next. For a common therapy to be effective, a full replacement of the coding sequence is often needed. As treatments for increasingly complex diseases are undertaken, multi-gene insertions will be necessary requiring larger cargos beyond the limits of current virus-based approaches. While a multitude of genetic edits are now possible through the use of CRISPR nuclease-induced non-homologous end-joining (NHEJ; deletion) and homology-directed repair (HDR; insertion), base editing (single base pair replacement), or prime editing (small insertion, replacement, or deletion), notably HDR insertion efficiency drops significantly for larger cargos and HDR is inefficient or nonfunctional in non-dividing cells which make up most therapeutically relevant cell types. NHEJ-based insertion methods exist but are generally inefficient and, like HDR, rely on unprotected double-strand breaks that can cause uncontrolled large deletions or chromosomal rearrangements. prime editing functions in both dividing and non-dividing cells and efficiently inserts sequences <50 base pairs while minimizing double stranded breaks. An improved version or prime editing, referred to as twin prime editing, has been developed, which uses the attachment (att) site from Bxb1 to insert cargos as large as 5.6 kb at the att site (Anzalone et al. (2022) Nat. Biotechnol. 40:731-740). This approach overcomes many of the hurdles of current gene insertion tools and holds great promise for correcting genetic diseases.

Nevertheless, there remains a need in the art to improve the efficiency of integrase-mediated integration. The present disclosure addresses this need in the art.

### SUMMARY

In one aspect is provided an evolved Bxb1 integrase comprising an amino acid sequence that is at least 90% identical to the amino acid sequence of SEQ ID NO:1, wherein the amino acid sequence of the evolved Bxb1 integrase comprises one or more mutations and exhibits at least a 2-fold increase in integration of exogenous nucleic acids into a genomic target site as compared to wild-type Bxb1 integrase

In another aspect, an evolved PhiC31 integrase is provided that comprises an amino acid sequence that is at least 90% identical to the amino acid sequence of SEQ ID NO:2, wherein the amino acid sequence of the evolved PhiC31 integrase comprises at least one mutation as described herein.

Also provided herein is a method of altering the sequence of a genome of a cell by contacting a cell with the evolved Bxb1 or PhiC31 integrase of the disclosure, wherein the cell includes in its genome at least one integrase recognition sequence recognized by the evolved integrase so that the evolved integrase binds to the integrase recognition sequence and alters the sequence of the genome of the cell.

### BRIEF DESCRIPTION OF THE DRAWINGS

FIG. 1. Amplicon sequencing comparison of twin prime editing using dual epegRNA at target sites AAVS1, Xq22.1, and human ROSA26 in HEK293 cells. Mean and s.d. bars represent 2 biological replicates transfected on separate days.

FIG. 2. Evolved integrase helper plasmids were co-transfected into the clonal HEK293 cell line containing a pre-installed attP landing pad with a 6.6 kb donor plasmid. Unevolved controls are shown for comparison. On-target integrations were measured with droplet digital PCR. n=3.

FIG. 3. Integration by PACE evolved PhiC31 mutants with twin prime editing at ROSA in HEK293 cells measured by droplet digital PCR. Unevolved controls are also shown. n=3.

FIG. 4. Comparison of twin prime editing and integration frequencies of 6.6 kb donor with PhiC31, evolved PhiC31, Pa01, and Bxb1 integrases in HEK293T cells measured by droplet digital PCR. n=3.

FIGS. 5A and 5B. Twin prime editing and integration frequency in HEK293 cells of PACE evolved Bxb1 mutants measured by droplet digital PCR. n≥4 on multiple days.

FIG. 6. Comparison of combination mutants on HEK293 ROSA attB cell line. Integration of a 6.6 kb donor plasmid measured by droplet digital PCR. n=4.

FIG. 7. Integration and twin-PE frequencies in HEK293T cells with selected PACE evolved clones and combination mutants. n=4 on two separate days.

FIG. 8. Optimized twin prime editing and integration in HEK293T. Cells were transfected and sorted 3 days post transfection for dsRed expression before lysis and measurement by droplet digital PCR. n=3.

FIG. 9. Optimized twin prime editing and integration in K562. Cells were transfected and sorted 3 days post transfection for dsRed expression before lysis and measurement by droplet digital PCR. n=3.

FIG. 10. Optimized twin prime editing and integration in HeLa. Cells were transfected and sorted 3 days post transfection for dsRed expression before lysis and measurement by droplet digital PCR. n=3.

FIG. 11. Droplet digital PCR detection of Human Factor VIII (FVIII) and Von Willebrand Factor (VWF) donor integration at ROSA26.

FIG. 12. Detection of secreted FVIII in culture media by ELISA and Factor X activation assay.

FIG. 13. Detection of secreted VWF in culture media by ELISA.

### DETAILED DESCRIPTION

Serine integrases such as PhiC31 and Bxb1 enable the insertion of large DNA cargos at attachment (att) sites. By targeting att sites to the genome using technologies such as prime editing (PE), integrases can deliver cargo to safe loci while avoiding double-strand breaks. Phage-assisted continuous evolution (PACE) was used to evolve the PhiC31 and Bxb1 serine integrases to increase their activity on their respective native attachment (att) sequence. The resulting sequence of the improved integrases was subcloned into mammalian expression vectors and tested for activity of insertion of large transgenes into the human genome of cell lines. The att site that each integrase uses for insertion was first inserted into the genome using twin prime editing. Next, each integrase inserted cargo DNA into the att site at a specific location in the genome. The efficiency was accurately measured using droplet digital PCR. During pre-optimized transfection conditions without sorting for transfected cells, the wild-type integrase inserted its cargo DNA at 3.7% of the haploid genomes. By comparison, the evolved integrases inserted their cargo DNA into up to 21.9% of haploid genomes. During optimized transfection conditions using cells sorted for complete transfection, the wild-type integrase inserted its cargo DNA at 32% of the haploid genomes. By comparison, the evolved integrases inserted their cargo DNA into up to 80% of haploid genomes following sorting. The improved integrases find use in replacing defective genes in the genome. By directing insertion to specific sites in the genome, the risk of off-target insertion and insertional mutagenesis is significantly reduced. The increase in efficiency afforded by the improved integrases described herein will dramatically improve the effectiveness of this technology as a therapy for treating a disease or disorder for which altering the sequence of a gene will provide a benefit.

Accordingly, some aspects provide evolved integrases that exhibit increased insertion activity and efficiency as compared to their wild-type counterparts and use of the evolved integrases to modify, for example, any sequence within the genome of a cell or subject. In particular, evolved Bxb1 or PhiC31 integrases are described that exhibit an increase in insertion efficiency as compared to wild-type Bxb1 or PhiC31 integrase. The term “wild-type (or native) integrase” refers to an integrase that is naturally occurring and/or has not been modified through human intervention.

The evolved Bxb1 and PhiC31 integrases described herein have at least one mutation, or at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 mutations. Mutations may include substitutions, deletions, or insertions without eliminating recombinase activity.

A wild-type Bxb1 integrase may have the following amino acid sequence:

(SEQ ID NO: 1)
MRALVVIRLSRVTDATTSPERQLESCQQLCAQRGWDVVGVAEDLD
VSGAVDPFDRKRRPNLARWLAFEEQPFDVIVAYRVDRLTRSIRHL
QQLVHWAEDHKKLVVSATEAHFDTTTPFAAVVIALMGTVAQMELE
AIKERNRSAAHFNIRAGKYRGSLPPWGYLPTRVDGEWRLVPDPVQ
RERILEVYHRVVDNHEPLHLVAHDLNRRGVLSPKDYFAQLQGREP
QGREWSATALKRSMISEAMLGYATLNGKTVRDDDGAPLVRAEPIL
TREQLEALRAELVKTSRAKPAVSTPSLLLRVLFCAVCGEPAYKFA
GGGRKHPRYRCRSMGFPKHCGNGTVAMAEWDAFCEEQVLDLLGDA
ERLEKVWVAGSDSAVELAEVNAELVDLTSLIGSPAYRAGSPQREA
LDARIAALAARQEELEGLEARPSGWEWRETGQRFGDWWREQDTAA
KNTWLRSMNVRLTFDVRGGLTRTIDFGDLQEYEQHLRLGSVVERL
HTGMS.

In one aspect is provided an evolved Bxb1 integrase having an amino acid sequence that is at least 70%, at least 80%, at least 90%, at least 95%, at least 97%, at least 98% or at least 99% identical to the amino acid sequence of SEQ ID NO:1, wherein the evolved Bxb1 integrase has at least one mutation and exhibits an increase in insertion efficiency as compared to wild-type Bxb1 integrase. In some aspects, the evolved Bxb1 integrase has between one and 8 mutations, e.g., between one and 3 mutations, between 2 and 4 mutations, between 3 and 5 mutations, between 4 and 6 mutations, between 5 and 7 mutations, between 6 and 8 mutations, between 2 and 7 mutations or between 4 and 8 mutations as compared to the wild-type Bxb1 integrase (e.g., SEQ ID NO:1). In other aspects, the evolved Bxb1 integrase has at least one mutation, at least 2 mutations, at least 3 mutations, at least 4 mutations, at least 5 mutations, at least 6 mutations, at least 7 mutations or at least 8 mutations as compared to the wild-type Bxb1 integrase (e.g., SEQ ID NO:1).

In further aspects, the evolved Bxb1 integrase has at least one mutation, the at least one mutation being at position 4, 5, 18, 24, 34, 36, 40, 42, 45, 46, 49, 50, 51, 57, 61, 62, 63, 67, 69, 70, 74, 75, 76, 79, 85, 87, 88, 89, 90, 92, 95, 99, 100, 105, 106, 110, 111, 119, 122, 130, 133, 137, 140, 145, 153, 156, 160, 164, 166, 168, 174, 175, 178, 179, 181, 187, 189, 191, 194, 203, 209, 218, 223, 229, 231, 239, 248, 251, 254, 261, 263, 264, 268, 272, 278, 280, 281, 282, 283, 285, 287, 288, 292, 295, 302, 306, 307, 311, 313, 319, 321, 328, 331, 332, 333, 334, 341, 347, 353, 355, 359, 360, 361, 362, 366, 369, 370, 375, 380, 387, 388, 397, 398, 405, 409, 411, 414, 415, 416, 419, 425, 426, 428, 434, 435, 444, 445, 453, 461, 463, 466, 468, 476, 479, 480, 483, 484, 487, 488, 489, 494, 496, and/or 499 relative to SEQ ID NO:1. In some aspects, the evolved Bxb1 integrase has at least one mutation, the at least one mutation being L4I, V5V, S18S, E24E, G34D, D36A, D36N, V40I, V40A, V40V, E42K, D45G, V46V, A49S, V50I, D51D, D51N, D51G, D51E, R57K, L61F, A62A, R63K, F67S, E69A, E69E, Q70H, V74I, I75V, V76I, R79R, R85R, I87L, I87V, R88R, H89G, H89Y, L90L, Q92H, Q92Q, H95Y, D99N, H100N, H100H, V105A, S106S, A110A, H111P, A119S, V122M, A130A, E133E, I137I, R140R, A145T, K153K, G156G, P160P, L164L, T166I, V168I, L174L, V175V, V175A, P178P, V179A, V179I, R181K, R181R, V187I, V187V, H189N, H189Y, Q191Q, N194D, H203Y, G209G, A218A, R223R, E229K, S231S, M239I, A248T, N251K, N251N, T254S, A261T, A261S, L263I, V264A, P268P, R272Q, L278L, A280T, A280A, E281E, L282L, V283V, T285A, R287R, R287H, A288V, A288T, P292P, P295P, L302L, V306V, V306A, C307C, A311V, K313R, K313K, R319K, R319G, H321P, H321N, S328S, F331S, P332H, K333K, H334R, A341D, A347E, V353I, D355N, D359N, D359D, D359A, A360V, A360T, E361E, R362K, V366I, A369P, A369E, A369T, A369S, G370G, V375V, V380I, L387L, T388M, R397R, A398S, A405A, R409H, A411V, A414A, A414V, A415S, R416K, E419E, A425T, A425A, R426K S428S, E434G, T435T, R444L, E445Q, T453I, R461R, T463I, V466M, V466V, V466L, G468D, G468G, F476F, L479I, Q480STOP, E483E, Q484K, R487R, del L488, G489G, R494S, R494R, H496N, and/or M499T relative to SEQ ID NO:1. In some aspects, the evolved Bxb1 integrase has a combination of mutations as described herein, e.g., the combination of mutations being any one of the combinations listed in Table 1 with SEQ ID NO:1 (wild-type Bxb1) as reference.

TABLE 1

| Combination of Mutations | Exemplary Combination of Mutations |
|---|---|
| H111X, E434X | H111P, E434G |
| G156X, A369X | G156G, A369P |
| R63X, R88X, A110X, P295X | R63K, R88R, A110A, P295P |
| D36X, R63X, P292X, L479X | D36A, R63K, P292P, L479I |
| V5X, S106X, E434X | V5V, S106S, E434G |
| D51X, H189X | D51D, H189N |
| L164X, H203X, Q480X | L164L, H203Y, Q480STOP |
| F67X, V187X, A261X | F67S, V187I, A261T |
| I87X, B14: B33V187X, A218X, E419X, | I87L, B14: B33V187V, A218A, E419E, |
| A425X, E483X | A425T, E483E |
| R63X, A119X, P295X, E361X, M499X | R63K, A119S, P295P, E361E, M499T |
| R63X, V122X, P295X | R63K, V122M, P295P |
| R63X, A347X | R63K, A347E |
| V40X, R63X, V122X, P295X, R319X, | V40I, R63K, V122M, P295P, R319K, |
| S428X | S428S |
| R63X, V122X, P295X, R487X | R63K, V122M, P295P, R487R |
| L278X, P295X, P332X, A369X, V380X | L278L, P295P, P332H, A369T, V380I |
| E42X, R63X, D99X, E133X, A145X, | E42K, R63K, D99N, E133E, A145T, |
| K153X, P295X, A369X, R494X | K153K, P295P, A369E, R494S |
| V40X, R63X, H89X, Q191X, P295X, | V40A, R63K, H89G, Q191Q, P295P, |
| L302X, C307C | L302L, C307C |
| V40X, R63X, P295X, R362X, G370X, | V40V, R63K, P295P, R362K, G370G, |
| G468X | G468G |
| G209X, A288X, A311X, A398X, | G209G, A288V, A311V, A398S, R416K, |
| R416X, T453X, H496X, E42X, G209X | T453I, H496N, E42K, G209G |
| A288X, A311X, A398X, R416X, T453X, | A288V, A311V, A398S, R416K, T453I, |
| H496X | H496N |
| S18X, R63X, E69X, I87X, P295X, | S18S, R63K, E69E, I87V, P295P, P332H, |
| P332X, H334X, A369X | H334R, A369E |
| S18X, E42X, R63X, E69X, E133X, | S18S, E42K, R63K, E69E, E133E, P295P, |
| P295X, P332X, H334X | P332H, H334R |
| P268X, A288X, A311X, D359X, T388X, | P268P, A288V, A311V, D359D, T388M, |
| T453X, V466X, H496X | T453I, V466V, H496N |
| A288X, A311X, R319X, A398X, R416X, | A288V, A311V, R319G, A398S, R416K, |
| T453X, H496X, E434G | T453I, H496N, E434G |
| E42X, A288X, A311X, A398X, R416X, | E42K, A288V, A311V, A398S, R416K, |
| T453X, H496X, E434X | T453I, H496N, E434G |
| H95X, R287X, K313X | H95Y, R287R, K313R |
| H95X, A280X | H95Y, A280T |
| H95X, V264X | H95Y, V264A |
| H95X, T254X | H95Y, T254S |
| V46X, H95X, V179X, R223X | V46V, H95Y, V179A, R223R |
| H95X, V264X, E434X | H95Y, V264A, E434G |
| H95X, A62X, V466X | H95Y, A62A, V466M |
| A280X, A360X | A280A, A360T |
| L61X, E434X | L61F, E434G |
| L174X, S231X, A288X, E434X, T463X | L174L, S231S, A288T, E434G, T463I |
| V40X, A130X, V283X, A288X, E434X, | V40V, A130A, V283V, A288T, E434G, |
| G468X | G468D |
| E229X, K313X, A405X, T453X | E229K, K313K, A405A, T453I |
| L90X, E229X, N251X, D359X, A360X, | L90L, E229K, N251K, D359N, A360T, |
| V375X, R494X | V375V, R494R |
| Q92X, V179X, R181X, E229X, M239X, | Q92Q, V179I, R181K, E229K, M239I, |
| A360X, R444X | A360T, R444L |
| E69X, I87X, N251X, H321X, D355X | E69A, I87L, N251N, H321P, D355N |
| D51X, I87X, R272X, G489X | D51N, I87L, R272Q, G489G |
| I87X, L488X | I87L, del L488 frameshift |
| I87X, V105X, H321X, S328X, R397X, | I87L, V105A, H321N, S328S, R397R, |
| A411X | A411V |
| R79X, I87X, Q484X | R79R, I87L, Q484K |
| I87X, I137X, F331X, R409X, L488X | I87L, I137I, F331S, R409H, del L488 |
| | frameshift |
| E69X, H95X, R461X | E69A, H95Y, R461R |
| H95X, L282X | H95Y, L282L |
| I87X, A369X, F476X | I87L, A369P, F476F |
| G34X, I87X, T166X, E229X, A369X, | G34D, I87L, T166I, E229K, A369P, |
| T435X, G489X | T435T, G489G |
| I87X, R88X, R140X, K333X, A369X, | I87L, R88R, R140R, K333K, A369P, |
| A414X, V466X, F476X | A414A, V466M, F476F |
| I87X, A369X, E434X | I87L, A369S, E434G |
| I87X, A369X, A414X, A425X, E434X | I87L, A369P, A414V, A425A, E434G |
| R85X, I87X, A248X, V306X, A369X, | R85R, I87L, A248T, V306V, A369P, |
| A415X, E434X | A415S, E434G |
| I87X, P160X, V175X, V187X, A218X, | I87L, P160P, V175V, V187V, A218A, |
| E281X, V353X, E419X, A425X, E483X | E281E, V353I, E419E, A425T, E483E |
| I87X, Q92X, P160X, V175X, P178X, | I87L, Q92H, P160P, V175V, P178P, |
| V187X, A218X, E281X, V353X, E419X, | V187V, A218A, E281E, V353I, E419E, |
| A425X, E483X | A425T, E483E |
| E24X, I87X, H100X, V187X, A218X, | E24E, I87L, H100N, V187V, A218A, |
| E419X, A425X, E483X | E419E, A425T, E483E |
| I87X, V187X, A218X, A369X, E419X, | I87L, V187V, A218A, A369E, E419E, |
| A425X, E483X | A425T, E483E |
| I87X, V187X, A218X, E419X, A425X, | I87L, V187V, A218A, E419E, A425T, |
| E434X, E483X | E434G, E483E |
| I87X, H95X | I87L, H95Y |
| I87X, V122X | I87L, V122M |
| I87X, A369X | I87L, A369P |
| I87X, E434X | I87L, E434G |
| H95X, V122X | H95Y, V122M |
| H95X, A369X | H95Y, A369P |
| H95X, E434X | H95Y, E434G |
| V122X, A369X | V122M, A369P |
| V122X, E434X | V122M, E434G |
| A369X, E434X | A369P, E434G |
| I87X, H95X, V122X | I87L, H95Y, V122M |
| I87X, H95X, A369X | I87L, H95Y, A369P |
| I87X, H95X, E434X | I87L, H95Y, E434G |
| I87X, V122X, A369X | I87L, V122M, A369P |
| I87X, V122X, E434X | I87L, V122M, E434G |
| I87X, A369X, E434X | I87L, A369P, E434G |
| H95X, V122X, A369X | H95Y, V122M, A369P |
| H95X, V122X, E434X | H95Y, V122M, E434G |
| H95X, A369X, E434X | H95Y, A369P, E434G |
| V122X, A369X, E434X | V122M, A369P, E434G |
| I87X, H95X, V122X, A369X | I87L, H95Y, V122M, A369P |
| I87X, H95X, V122X, E434X | I87L, H95Y, V122M, E434G |
| I87X, H95X, A369X, E434X | I87L, H95Y, A369P, E434G |
| I87X, V122X, A369X, E434X | I87L, V122M, A369P, E434G |
| H95X, V122X, A369X, E434X | H95Y, V122M, A369P, E434G |
| I87X, H95X, V122X, A369X, E434X | I87L, H95Y, V122M, A369P, E434G |
| L4X, R63X, I87X, H95X, H111X, | L4I, R63K, I87L, H95Y, H111P, V122M, |
| V122X, A280X, A369X, E434X, V466X | A280T, A369P, E434G, V466M |
| R63X, V122X | R63K, V122M |
| H89X, A360X | H89Y, A360V |
| R181X, A341X | R181R, A341D |
| R287X, A425X | R287H, A425A |
| V50X, V306X | V50I, V306A |
| V366X, R426X | V366I, R426K |
| D359X, L387X | D359A, L387L |
| R57X, R63X, P295X | R57K, R63K, P295P |
| R63X, I87X, H100X, N194X, P295X, | R63K, I87V, H100H, N194D, P295P, |
| C307X | C307C |

X may be any amino acid.

A wild-type PhiC31 integrase may have the following amino acid sequence:

(SEQ ID NO: 2)
MDTYAGAYDRQSRERENSSAASPATQRSANEDKAADLQREVERDG
GRFRFVGHFSEAPGTSAFGTAERPEFERILNECRAGRLNMIIVYD
VSRFSRLKVMDAIPIVSELLALGVTIVSTQEGVFRQGNVMDLIHL
IMRLDASHKESSLKSAKILDTKNLQRELGGYVGGKAPYGFELVSE
TKEITRNGRMVNVVINKLAHSTTPLTGPFEFEPDVIRWWWREIKT
HKHLPFKPGSQAAIHPGSITGLCKRMDADAVPTRGETIGKKTASS
AWDPATVMRILRDPRIAGFAAEVIYKKKPDGTPTTKIEGYRIQRD
PITLRPVELDCGPIIEPAEWYELQAWLDGRGRGKGLSRGQAILSA
MDKLYCECGAVMTSKRGEESIKDSYRCRRRKVVDPSAPGQHEGTC
NVSMAALDKFVAERIFNKIRHAEGDEETLALLWEAARRFGKLTEA
PEKSGERANLVAERADALNALEELYEDRAAGAYDGPVGRKHFRKQ
QAALTLRQQGAEERLAELEAAEAPKLPLDQWFPEDADADPTGPKS
WWGRASVDDKRVFVGLFVDKIVVTKSTTGRGQGTPIEKRASITWA
KPPTDDDEDDAQDGTEDVAA

In one aspect is provided an evolved PhiC31 integrase having an amino acid sequence that is at least 70%, at least 80%, at least 90%, at least 95%, at least 97%, at least 98% or at least 99% identical to the amino acid sequence of SEQ ID NO:2, wherein the evolved PhiC31 integrase has at least one mutation and exhibits an increase in insertion efficiency as compared to wild-type PhiC31 integrase. In some aspects, the evolved PhiC31 integrase has between one and 13 mutations, e.g., between one and 3 mutations, between 2 and 4 mutations, between 3 and 5 mutations, between 4 and 6 mutations, between 5 and 7 mutations, between 6 and 8 mutations, between 7 and 9 mutations, between 8 and 10 mutations, between 9 and 11 mutations, between 10 and 12 mutations or between 11 and 13 mutations as compared to the wild-type PhiC31 integrase (e.g., SEQ ID NO:2). In other aspects, the evolved PhiC31 integrase has at least one mutation, at least 2 mutations, at least 3 mutations, at least 4 mutations, at least 5 mutations, at least 6 mutations, at least 7 mutations, at least 8 mutations, at least 9 mutations, at least 10 mutations, at least 11 mutations, at least 12 mutations, or at least 13 mutations as compared to the wild-type PhiC31 integrase (e.g., SEQ ID NO:2).

In further aspects, the evolved PhiC31 integrase has at least one mutation, the at least one mutation being at position 1, 2, 12, 14, 18, 24, 32, 36, 41, 43, 44, 45, 49, 51, 55, 58, 74, 77, 96, 103, 107, 117, 153, 176, 196, 197, 199, 200, 228, 229, 230, 231, 235, 238, 240, 252, 254, 255, 260, 262, 264, 266, 269, 274, 278, 302, 319, 320, 322, 331, 333, 340, 344, 345, 346, 347, 351, 353, 355, 359, 362, 364, 378, 382, 393, 396, 397, 399, 406, 410, 424, 429, 431, 436, 438, 445, 448, 449, 450, 452, 457, 468, 472, 478, 498, 499, 501, 505, 512, 514, 516, 517, 520, 527, 535, 536, 549, 551, 552, 553, 563, 568, 580, 585, 586, 587, 590, 592, 594, 600, 603, 604, 609, 612, and/or 616 relative to SEQ ID NO:2. In some aspects, the evolved PhiC31 integrase has at least one mutation, the at least one mutation being M1E, M1V, D2V, D2M, S12N, E14G, S18N, A24A, D32A, D36A, V41I, R43R, D44A, G45G, R49R, V51M, S55S, P58P, I74I, E77E, R96R, I103I, S107S, V117V, I153I, I153V, E176D, N196N, K197K, A199T, H200H, H228Y, L229L, P230S, F231L, S235S, A238A, H240R, D252G, D254D, A255G, G260G, T262T, G264G, K266K, S269N, P274S, M278L, T302A, L319L, R320R, V322V, E331E, A333T, A333S, A333D, A340V, G344V, G344D, R345R, G346S, R347K, L351V, L351Q, R353R, Q355Q, S359S, D362N, D362G, L364M, E378K, K382K, V393V, S396N, S396R, A397T, G399G, N406S, A410T, I424I, G429S, E431V, L436L, W438R, G445G, W448R, E449D, A450D, E452K, R457R, L468L, E472D, R478R, A498A, L499L, L501L, G505V, G505G, G505S, E512K, E514E, A516T, E517E, K520K, F527F, P535L, P535P, T536A, D549D, R551R, V552V, F553F, V563V, T568T, A580V, A585A, K586Q, P587P, D590G, D592G, D594N, D594D, T600S, T600T, V603I, V603A, V603V, A604S, P609S, V612V, and/or A616V relative to SEQ ID NO:2. In some aspects, the evolved PhiC31 integrase has a combination of mutations, the combination of mutations being any one of the combinations listed in Table 2 with wild-type PhiC31 (SEQ ID NO:2) as reference.

TABLE 2

| Combination of Mutations | Exemplary Combinations of Mutations |
|---|---|
| M1X, D2X, V41X | M1E, D2V, V41I |
| D32X, D36X, D44X | D32A, D36A, D44A |
| M1X, D2X, D32X, D36X, V41X, D44X | M1V, D2V, D32A, D36A, V41I, D44A |
| M1X, D2X, V41X, R49X, M278X, | M1V, D2V, V41I, R49R, M278L, L319L, |
| L319X, F527X | F527F |
| M1X, D2X, V41X, L229X, G344X | M1V, D2V, V41I, L229L, G344V |
| M1X, D2X, V41X, E431X | M1V, D2V, V41I, E431V |
| M1X, D2X, V41X, R43X, P230X, | M1V, D2V, V41I, R43R, P230S, Q355Q, |
| Q355X, K520X | K520K |
| M1X, D2X, V41X, V117X, H228X, | M1V, D2V, V41I, V117V, H228Y, |
| A255X, A580X, D594X | A255G, A580V, D594D |
| M1X, D2X, V41X, G505X, P587X, | M1V, D2V, V41I, G505V, P587P, |
| D594X | D594N |
| D32X, A199X, P535X | D32A, A199T, P535P |
| D32X, D36X, D44X, S396X, R551X | D32A, D36A, D44A, S396N, R551R |
| D32X, D36X, D44X, N406X, E512X | D32A, D36A, D44A, N406S, E512K |
| D32X, D36X, D44X, K197X, D254X, | D32A, D36A, D44A, K197K, D254D, |
| G344X, A397X, R457X, A616X | G344D, A397T, R457R, A616V |
| D32X, D36X, D44X, S55X, E77X, | D32A, D36A, D44A, S55S, E77E, I153I, |
| I153X, G344X, S359X, V552X, A585X | G344D, S359S, V552V, A585A |
| D32X, D36X, D44X, K266X, D362X | D32A, D36A, D44A, K266K, D362G |
| D32X, D36X, D44X, F231X, G505X, | D32A, D36A, D44A, F231L, G505S, |
| D592X | D592G |
| D32X, D36X, D44X, I103X, T302X, | D32A, D36A, D44A, I103I, T302A, |
| D362X, G399X, E449X, E472X | D362G, G399G, E449D, E472D |
| D32X, D36X, D44X, E77X, I153X, | D32A, D36A, D44A, E77E, I153I, |
| H200X, P274X, G344X, K382X, T536X | H200H, P274S, G344D, K382K, T536A |
| D32X, D36X, D44X, R320X, A333X, | D32A, D36A, D44A, R320R, A333T, |
| G429X, A516X, A604X | G429S, A516T, A604S |
| D32X, D36X, D44X, A238X, R457X, | D32A, D36A, D44A, A238A, R457R, |
| P535X | P535L |
| E14X, D32X, D36X, D44X, E176X, | E14G, D32A, D36A, D44A, E176D, |
| A238X, S396X, R478X, F553X, K586X | A238A, S396R, R478R, F553F, K586Q |
| D32X, D36X, D44X, E176X, L229X, | D32A, D36A, D44A, E176D, L229L, |
| A238X, A333X, S359X | A238A, A333D, S359S |
| D32X, D36X, D44X, I74X, R96X, | D32A, D36A, D44A, I74I, R96R, E176D, |
| E176X, A238X, A333X, E378X, D590X | A238A, A333D, E378K, D590G |
| M1X, D2X, D32X, D36X, V41X, D44X | M1E, D2V, D32A, D36A, V41I, D44A |
| M1X, D2X, D32X, D36X, V41X, D44X, | M1E, D2V, D32A, D36A, V41I, D44A, |
| S235X, L364X | S235S, L364M |
| M1X, D2X, D32X, D36X, V41X, D44X, | M1E, D2V, D32A, D36A, V41I, D44A, |
| G346X, G505X | G346S, G505G |
| M1X, D2X, D32X, D36X, V41X, D44X, | M1E, D2V, D32A, D36A, V41I, D44A, |
| R345X, D362X, L468X, V603X | R345R, D362N, L468L, V603I |
| M1X, D2X, D32X, D36X, V41X, D44X, | M1E, D2V, D32A, D36A, V41I, D44A, |
| D362X, V612X | D362N, V612V |
| M1X, D2X, A24X, D36X, V41X, D44X, | M1E, D2V, A24A, D36A, V41I, D44A, |
| N196X, W448X, P609X | N196N, W448R, P609S |
| M1X, D2X, D32X, D36X, V41X, D44X, | M1E, D2V, D32A, D36A, V41I, D44A, |
| D362X, V563X, T600X | D362G, V563V, T600T |
| M1X, D2X, D32X, D36X, V41X, D44X, | M1E, D2V, D32A, D36A, V41I, D44A, |
| F231X, A410X, V603X | F231L, A410T, V603V |
| M1X, D2X, D32X, D36X, V41X, D44X, | M1E, D2V, D32A, D36A, V41I, D44A, |
| T262X, D362X, V393X, L436X | T262T, D362N, V393V, L436L |
| M1X, D2X, D32X, V41X, D44X, G45X, | M1E, D2M, D32A, V41I, D44A, G45G, |
| G264X, E331X, L351X, G445X, V563X, | G264G, E331E, L351V, G445G, V563V, |
| A580X, V603X | A580V, V603V |
| M1X, D2X, A24X, D32X, D36X, V41X, | M1E, D2V, A24A, D32A, D36A, V41I, |
| D44X, W438X, P609X | D44A, W438R, P609S |
| M1X, D2X, A24X, D32X, D36X, V41X, | M1E, D2V, A24A, D32A, D36A, V41I, |
| D44X, H240X, A333X, A340X, W448X, | D44A, H240R, A333S, A340V, W448R, |
| T600X, P609X | T600S, P609S |
| M1X, D2X, D32X, D36X, V41X, D44X, | M1E, D2V, D32A, D36A, V41I, D44A, |
| R347X, I424X | R347K, I424I |
| M1X, D2X, D32X, D36X, V41X, D44X, | M1E, D2V, D32A, D36A, V41I, D44A, |
| V322X, L351X, D549X | V322V, L351Q, D549D |
| M1X, D2X, D32X, D36X, V41X, D44X, | M1E, D2V, D32A, D36A, V41I, D44A, |
| S107X, D362X | S107S, D362N |
| M1X, D2X, S12X, D32X, D36X, V41X, | M1E, D2V, S12N, D32A, D36A, V41I, |
| D44X, P58X, I103X, E153X, E514X, | D44A, P58P, I103I, E153V, E514E, |
| A580X, V603X | A580V, V603A |
| M1X, D2X, D32X, D36X, V41X, D44X, | M1E, D2V, D32A, D36A, V41I, D44A, |
| R353X, D362X, L499X, T568X | R353R, D362N, L499L, T568T |
| M1X, D2X, S18X, D32X, D36X, V41X, | M1E, D2V, S18N, D32A, D36A, V41I, |
| D44X, V51X, I103X, I153X, A450X, | D44A, V51M, I103I, I153V, A450D, |
| V603X | V603A |
| M1X, D2X, D32X, D36X, V41X, D44X, | M1E, D2V, D32A, D36A, V41I, D44A, |
| I103X, I153X, S269X, A450X, E517X, | I103I, I153V, S269N, A450D, E517E, |
| V603X | V603A |
| M1X, D2X, D32X, D36X, V41X, D44X, | M1E, D2V, D32A, D36A, V41I, D44A, |
| I103X, I153X, G260X, L436X, A450X, | I103I, I153V, G260G, L436L, A450D, |
| L501X, V603X | L501L, V603A |
| M1X, D2X, D32X, D36X, V41X, D44X, | M1E, D2V, D32A, D36A, V41I, D44A, |
| I103X, I153X, A450X, E452X, V603X | I103I, I153V, A450D, E452K, V603A |

X may be any amino acid.

In some aspects, the evolved PhiC31 integrase may further include one or more of the following mutations: M1E, D2V, D32A, D36A, V41I, and/or D44A with respect to the wild-type sequence (SEQ ID NO:2). In other aspects, the evolved PhiC31 further includes the combination of V41I; D32A, D36A and D44A; or D32A, D36A, V41I, and D44A.

In still further aspects, the evolved PhiC31 integrase may have the N-terminal sequence as in SEQ ID NO:2 or may further include the N-terminal amino acid sequences listed in Table 3, which replace the methionine at position 1 of SEQ ID NO:2.

TABLE 3

| | SEQ |
|---|---|
| | ID |
| N-terminal Sequence | NO: |
| MIQGVVSG | 3 |
| MIQEVVSG | 4 |
| MTMITPSAQLTLTKGNKSWSSLVTAASVLEFATMIQGVAG | 5 |
| MTMIYPFPAQLTLTKGNKSWSSLVTAASVLEFATMIQGVAG | 6 |
| MTMITPSAQLTLTKSNKSWNSLVTAASVLEFATMIQGVAG | 7 |
| MTMITPSAQLTLTKGNKSWSSLVTAASVLEFATMIQGVAG | 8 |
| MTMITPSAQLTLTKSNKSWSSLVTAASVLEFATMIQGVTG | 9 |
| MTMITPSAQLTLTKDNKSWSSLVTAASVLEFATMIQGVAG | 10 |
| MTMITPSAQLTLTKSNKSWSSLVTAASVLEFATMIQGVAG | 11 |
| MTMITPSAQLTLTKSNKSWSSLVTAASVLEFATMIQGVAG | 12 |

In some aspects, one or more of the mutations in the evolved Bxb1 and PhiC31 integrases are amino acid substitutions. Substitutions, for example, those designated herein, e.g., in Tables 1 and 2, may be conservative or nonconservative, i.e., amino acid residues designated as “X” may conservative or nonconservative substitutions of the wild-type amino acid residue at the indicated position. In contrast to a nonconservative amino acid substitution, a conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine, and histidine; interchangeable acidic amino acids are aspartic acid and glutamic acid; and interchangeable small amino acids are alanine, serine, threonine, cysteine, and glycine. A mutation within the context of this disclosure may also include “silent mutations” that do not result in changes to the amino acid sequence.

In some aspects, the evolved Bxb1 integrase or evolved PhiC31 integrase described herein, referred to generally as the “evolved integrase,” recognizes (i.e., binds) an integrase recognition sequence that comprises a naturally occurring sequence, i.e., its canonical integrase recognition sequence. In some aspects, an evolved integrase recognizes an integrase recognition sequence that is in the genome of a mammal. In some aspects, the evolved integrase recognizes an integrase recognition sequence in the genome of a human. In some aspects, the evolved integrase recognizes an integrase recognition sequence that occurs only once in the genome of a mammal. In some aspects, the evolved integrase recognizes an integrase recognition sequence in the genome of a mammal that differs from any other site in the genome by at least 1, at least 2, at least 3, at least 4, at least 5, at least 6, at least 7, at least 8, at least 9, at least 10, at least 11, at least 12, at least 13, at least 14, or at least 15 nucleotide(s). Variants may share at least 65%, 70%, 75%, 80%, 85%, 90%, 91%, 92%, 93%, 94%, 95%, 96%, 97%, 98%, 99% or more sequence identity to the native recognition sequence, wherein the variants retain biological activity and hence facilitate a recombination event in the presence of the appropriate integrase. Assays to measure the biological activity of recombination sites are known in the art. See, for example, Senecoll et al. (1988) J. Mol. Biol. 201:406-421; Voziyanov et al. (2002) Nucleic Acid Research 30:7; U.S. Pat. No. 6,187,994; and Albert et al. (1995) The Plant Journal 7:649-659.

In some aspects, the evolved integrase recognizes (i.e., binds) an integrase recognition sequence located in a safe harbor genomic locus. In some aspects, the safe harbor genomic locus is a Rosa26 locus. In other aspects, the safe harbor genomic locus is an AAVS1 locus. In further aspects, the safe harbor genomic locus is an Xq22.1 locus. In some aspects, the evolved integrase recognizes an integrase recognition sequence located in a genomic locus associated with a disease or disorder. In other aspects, the present disclosure provides the use of prime editing, e.g., twin prime editing, to install one or more integrase recognition sequences into the genome at a specified location, such as a safe harbor locus, depending on the desired application. Introduction of two integrase recognition sequences could result in deletion of the intervening sequence, inversion of the intervening sequence, chromosomal translocation, or cassette exchange, depending on the identity and orientation of the targets. By choosing endogenous sequences that already closely resemble integrase recognition sequences, the scope of editing required to introduce the complete integrase recognition sequence would be reduced. Examples of integrase recognition sequences of use in combination with the evolved Bxb1 integrase described herein include, but are not limited to, those having the nucleotide sequence of SEQ ID NO:13-14. Examples of integrase recognition sequences of use in combination with the evolved PhiC31 integrase described herein include, but are not limited to, those having the nucleotide sequence of SEQ ID NO:15-16.

In other aspects, the evolved integrases exhibit at least a 2-fold, preferably at least 3-fold, at least 4-fold, at least 5-fold, at least 6-fold, at least 7-fold, at least 8-fold, at least 9-fold, or at least 10-fold improvement in integration of cargo DNA into a target site as compared to a wild-type integrase (e.g., SEQ ID NO:1 or SEQ ID NO:2).

In some aspects, nucleic acids (RNA, DNA, or a combination thereof) encoding any of the evolved integrases described herein are provided. In some aspects, the nucleic acids encoding the integrases are under the control of a heterologous promoter. In some aspects, the encoding nucleic acids are included in an expression construct, e.g., a plasmid, a viral vector, or a linear expression construct. In some aspects, the nucleic acid or expression construct is in a cell, tissue, or organism.

Nucleic acids encoding any of the evolved integrases described herein, may be in any number of nucleic acid “vectors” known in the art. As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Nonviral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also include the entire genome sequence or recombinant genome sequence of a virus. A vector can also include a portion of the genome that includes the functional sequences for production of a virus capable of infecting, entering, or being introduced to a cell to deliver nucleic acid therein.

Expression of any of the integrases described herein may be controlled by any regulatory sequence (e.g., a promoter sequence) known in the art. Regulatory sequences, as described herein, are nucleic acid sequences that regulate the expression of a nucleic acid sequence. A regulatory or control sequence may include sequences that are responsible for expressing a particular nucleic acid (e.g., a nucleic acid encoding an integrase) or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell, and introns. A heterologous regulatory region is not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

The term “operably linked” refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken β-actin (CBA) promoter, and a hybrid form of the CBA promoter (CBh).

Some aspects of this disclosure provide for delivery of the evolved integrases described herein (e.g., as an isolated protein or via nucleic acids encoding the evolved integrase) to host cells. Some aspects provide host cells expressing an evolved integrase provided herein, e.g., by virtue of harboring nucleic acids and/or an expression construct encoding the evolved integrase. In some aspects, cells contacted with an evolved integrase (e.g., protein or nucleic acids encoding the evolved integrase) as described herein are provided, e.g., cells contacted in vitro, in vivo, or ex vivo. In some aspects, the cell is a prokaryotic cell, for example, a bacterial cell. In some aspects, the cell is an E. coli cell. In some aspects, the cell is a eukaryotic cell, for example, a yeast cell, an insect cell, or a mammalian cell, e.g., hamster host cell, a human host cell, a rat host cell, or a mouse host cell. In some aspects, a host cell is a Chinese hamster ovary (CHO) host cell, a CHO K1 host cell, a CHO K1SV host cell, a DG44 host cell, a DUKXB-11 host cell, a CHOK1S host cell, a CHO K1M host cell, a monkey kidney CV1 line transformed by SV40 (COS-7), human embryonic kidney line (293 or HEK293T), baby hamster kidney cells (BHK), mouse sertoli cells (TM4 cells), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor (MMT 060562), TRI cells, MRC5 cells, FS4 cells, YO cells, NSO cells, Sp2/0 cells, or PER.C6® cells.

In some aspects, a host cell is a cell line that has been cultured for a certain number of generations. In certain aspects, a host cell is a primary cell. In some aspects, a host cell is a T cell.

Some aspects further provide pharmaceutical compositions comprising an evolved integrase as provided herein and a pharmaceutically acceptable carrier. In some aspects, the pharmaceutical composition is formulated for administration to a subject. In some aspects, the pharmaceutical composition comprises an effective amount of the evolved integrase for recombining an integrase recognition sequence in a cell of a subject in vivo, ex vivo, or in vitro. In some aspects, the composition further comprises a nucleic acid molecule comprising at least one integrase recognition sequence adjacent to a sequence to be inserted into a genetic locus within the genome of the subject.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative, and antioxidants can also be present in the formulation. The terms such as “excipient”, “carrier”, “pharmaceutically acceptable carrier” or the like are used interchangeably herein. In some aspects, the pharmaceutical composition is formulated for delivery to a subject, e.g., for gene editing.

The evolved integrases may be provided or packaged as a unit dose. The term “unit dose” when used in reference to a pharmaceutical composition refers to physically discrete units suitable as unitary dosage for the subject, each unit containing a predetermined quantity of active material calculated to produce the desired therapeutic effect in association with the required diluent, i.e., a carrier or vehicle.

In some aspects, a method of altering the sequence of a genome of a cell is provided. In accordance with this method, a cell is contacted with an evolved integrase described herein (e.g., an evolved Bxb1 or PhiC31 integrase protein or a nucleic acid molecule encoding an evolved Bxb1 or PhiC31 integrase), either in vivo, ex vivo, or in vitro, wherein the cell comprises in its genome at least one integrase recognition sequence recognized by the evolved integrase so that the evolved integrase binds to the integrase recognition sequence and alters the sequence of the genome of the cell. In some aspects, the alteration or change in the genome is a transversion, transition, deletion, or insertion. In some aspects, the alteration or change in the genome includes an excision of a sequence associated with a disease or disorder, such as, for example, a mutated version of a gene, or an integrated viral genome. In some aspects, the alteration or change in the genome may be a mutation-correcting change to a nucleotide sequence, insertion of a protein or RNA tag, insertion of immunoepitopes on proteins of interest, insertion of inducible dimerization domains in proteins, and/or introduce or remove sequences to alter that activity of a biomolecule, In some aspects, the contacting results in the integration of an exogenous nucleic acid sequence into the genome of the cell, e.g., at a safe harbor locus (e.g., Rosa26, AAVS1 or Xq22.1 locus) or in place of an undesired sequence.

In some aspects, the evolved integrases are used in combination with prime editing (PE), twin prime editing or multi-flap prime editing approaches to genome editing. See, e.g., PCT/US2021/31439 and PCT/US2022/078655. Twin prime editing and multi-flap prime editing involve the installation of one or more site specific integrase recognition sequences in a target genomic locus (e.g., a specific gene, exon, intron, or regulatory sequence of a target DNA, target gene, target genome, or target cell). The integrated one or more site-specific integrase recognition sequences may then undergo site-specific recombination in the presence of the integrase to effectuate a large-scale genetic alteration or change, such as an insertion, deletion, inversion, replacement, and/or chromosomal translocation.

In some aspects, insertion of a two or more integrase recognition sites can be accomplished with either prime editing having single PEgRNAs (multiplexed single flap prime editing) or twin prime editing. For example, in some aspects, a prime editor system including a single PEgRNA directs the prime editor system to introduce two or more integrase recognition sites in a target DNA. In some aspects, a prime editor system includes two or more PEgRNAs, wherein each of the two or more PEgRNAs include a DNA synthesis template that independently include an integrase recognition site. For example, a prime editor system may include a first PEgRNA and a second PEgRNA, wherein the first PEgRNA includes a first spacer that is complementary to a first target region in a target DNA, and a first DNA synthesis template that includes a first integrase recognition site, and wherein the second PEgRNA includes a second spacer that is complementary to a second target region in a target DNA, and a second DNA synthesis template that includes a second integrase recognition site, and wherein the first target region and the second target region are in different positions in the target DNA.

Integrase recognition sites introduced by prime editing can be used to generate an intended edit, including deletions, insertions, integrations, and replacement by donor sequences. In some aspects, an insertion interrupts the expression of a protein encoding gene, wherein the interruption of expression confers a therapeutic benefit in a cell. In some aspects, the insertion interrupts the expression of a protein coding gene, wherein the protein coding gene has deleterious effect to the cell or the subject, for example, a gene having a gain of function mutation. In some aspects, the insertion is at a genomic location that allows expression of the DNA sequence that is inserted. For example, in some aspects, the insertion is directly downstream of an endogenous gene promoter.

In some aspects, the inserted sequence encodes a protein, or is a portion of a gene that encodes a protein. For example, in some aspects, a target gene includes a 5′ portion and a 3′ portion, where the 3′ portion includes a mutation associated with a disease. In some aspects, the inserted DNA sequence can be downstream of the 5′ portion, e.g., at the 3′ end of an endogenous exon, wherein the inserted DNA sequence has a wild-type sequence corresponding to the 3′ portion of the gene, thereby restoring wild-type sequence of the gene. In some aspects, the inserted DNA sequence includes a stop codon at the 3′ end. In some aspects, prime editing with an evolved integrase results in replacement of an endogenous sequence in a target DNA by an exogenous nucleic acid sequence, or replacement of a target gene by an exogeneous nucleic acid sequence.

For example, in some embodiments, the target DNA in the genome of a cell is a target gene that includes one or more mutations associated with a disease. In some embodiments, a fragment of the target gene that includes the one or more mutations can be replaced with an exogenous nucleic acid sequence that has wild-type sequence of the same gene corresponding to the endogenous portion that comprises the one or more disease associated mutations.

In some aspects, the inserted exogenous nucleic acid sequence encodes a therapeutic protein. In some aspects, the inserted exogenous nucleic acid sequence is a therapeutic gene or a portion of the therapeutic gene that encodes a therapeutic protein, wherein the target genome includes one or more endogenous copies of the same gene that has a mutation. In some aspects, the mutation is a loss of function mutation, and expression of the inserted exogenous nucleic acid sequence restores or partially restores wild-type expression of the protein. For example, the therapeutic protein is Factor VIII. In some aspects, the therapeutic protein is adult hemoglobin or fetal hemoglobin. Other therapeutic genes encoding therapeutic proteins include genes encoding, e.g., steroids, growth factors, insulin, neurotransmitters, dopamine, norepinephrine, epinephrine, histamine, serotonin, checkpoint inhibitor proteins, antibodies, antigen receptor polypeptides, cell surface marker recognition polypeptides, a chimeric antigen receptor (CAR), a T-cell receptor, a protein involved in protein process (e.g., a degron or ubiquitin). Other sequences that may be inserted include a tissue specific promoters, enhancers, repressors, or insulators.

In some aspects, the evolved integrases are used in the treatment of a disease or disorder, i.e., site-specific recombination via the evolved integrase effectuates a large-scale genetic alteration or change to delay the development or progression of the disease or reduce disease severity. Treating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

Many large-scale genomic alterations or changes, such as gene insertions, deletions, inversions, replacements, or chromosomal translocations, are implicated in genetic disease. For example, microdeletions of chromosomes can lead to disease, and replacement of these deletions by insertions of critical DNA elements could lead to a permanent amelioration of disease. In addition, diseases resulting from inversions, gene copy number changes, or chromosomal translocations could be addressed by restoring the previous gene structure in affected cells. Examples of genetic diseases linked to large-scale genomic modifications that may be treated using the evolved integrases include, but are not limited to, Trisomy 17p (a gene duplication), Charcot-Marie-Tooth disease type I (a gene duplication), Smith-Magenis syndrome (a gene deletion), Williams-Beuren syndrome (a gene deletion), De la Chapelle syndrome (a chromosomal translocation), some forms of Down Syndrome (a chromosomal translocation), Hemophilia A (a gene inversion), and Hunter syndrome (a gene inversion).

The following non-limiting examples are provided to further illustrate the present invention.

### Example 1: Materials and Methods

General Methods. Oligonucleotides and synthetic DNA fragments were purchased from Integrated DNA Technologies. PCR amplification of DNA fragments was performed with KOD One™ PCR Mastermix (Toyobo). Plasmids were constructed using NEBuilder® HiFi DNA Assembly or USER® cloning (New England Biolabs) and purified with the Zyppy™ Plasmid Miniprep Kit (Zymo). DNA for transfection was purified by Zymopure™ Plasmid Miniprep Kit (Zymo), Zymopure™ Express Plasmid Midiprep Kit (Zymo), or Qiagen® Plasmid Plus Midiprep Kit (Qiagen).

Mammalian expression plasmids. Helper plasmids for mammalian expression called pCMV2 PhiC31 wt, pCMV2 HuOpt Bxb1, and pCMV2 HuOpt Pa01 encode wild-type PhiC31, Bxb1, and Pa01 integrases, respectively. The wild-type protein sequences for Bxb1 and PhiC31 are provided herein as SEQ ID NO:1 and SEQ ID NO:2, respectively. Hyperactive mutations in PhiC31 and Bxb1 integrases were incorporated into pCMV2 PhiC31 wt, and pCMV2 HuOpt Bxb1 for use in genome targeting experiments with mammalian cells. Hyperactive mutations originally identified by Keravala et al ((2009) Mol. Ther. 17(1):112-20) were also incorporated into pCMV2 PhiC31 wt to generate plasmids called pCMV2 PhiC31 P1, pCMV2 PhiC31 P2, and pCMV2 PhiC31 P3. Donor plasmids contained an att site and cargo DNA for genomic insertion. Donor plasmids pCMV PhiC31 attB DsRed Hygro, pCMV Bxb1 attP DsRed Hygro, and pCMV Pa01 attP DsRed Hygro contained att sites for PhiC31, Bxb1, and Pa01 integrases, respectively. The donor plasmids each contained the DsRed fluorescent protein and hygromycin resistance gene. In addition to DsRed and hygromycin, donor plasmids pCDNA4 FVIII Bxb1 attP HIR Donor and pCDNA3.1-WT-VWF Bxb1 attP HIR Donor contained the therapeutic Factor VIII and Von Willebrand Factor (vWF) genes, respectively. The PE5max plasmid encodes an S. pyogenes Cas9 (SpCas9) nickase fused to a reverse transcriptase used for PE (Chen et al. (2021) Cell 184:5635-5652). Plasmids used for expressing the epegRNA included a spacer sequence that directs the SpCas9 nickase to the genomic target sequence (Anzalone et al. (2022) Nat. Biotechnol. 40:731-740; Nelson et al. Nat. Biotechnol. 402-410). The epegRNA expression plasmids included a reverse transcription template (RTT) for the att site that became incorporated at the target sequence by PE. The epegRNA expression plasmids were used for PE insertion of the att site at human genomic target sequences AAVS1, Xq22.1, and ROSA26. Helper, donor and epegRNA plasmids will be made available through Addgene.

Mammalian Cell Culture. HEK293 (ATCC CRL-1573) and HEK293T (Thermo Fisher Scientific) cells were maintained in Dulbecco's Modified Eagle Medium (DMEM)+GlutaMAX™ (Gibco), HeLa cells (Sigma) were maintained in Eagle's Minimum Essential Medium (EMEM) with L-glutamine (ATCC), and K562 cells (Sigma) were maintained in RPMI-1640 medium (Sigma). All media were supplemented with 10% heat inactivated fetal bovine serum (FBS; Neuromics), lx Penicillin/Streptomycin (Gibco), and 0.75 μg/mL Amphotericin B (Gibco). Phosphate buffered saline (PBS), trypsin, and Opti-MEM™ were purchased from Gibco. Primary hepatocytes (Xenotech) were thawed using the OptiThaw Hepatocyte Kit, plated in OptiPlate medium, and cultured in OptiCulture medium (Xenotech).

Transfection. HEK293, HEK293T, and K562 cells were transfected with TransIT®-2020 (Mirus Bio). Primary hepatocytes were transfected with TransIT®-X2 dynamic (Mirus Bio). HeLa cells were transfected with HeLaMONSTER® (Mirus Bio).

Fluorescence-Activated Cell Sorting (FACS). All donor plasmids constitutively expressed DsRed. For experiments requiring sorting of transfected cells and exclusion of non-transfected cells, including select HEK293T transfections as well as Hela and K562 transfections, a FACSAria™ IIu flow cytometer (BD Biosciences) was used to sort cells based on DsRed fluorescent intensity (excitation 488 nm, emission 610 nm). Cells were immediately pelleted and lysed for droplet digital PCR analysis.

Cell Lysis. Cell pellets were lysed by addition of 90 μl DirectPCR® Cell lysis reagent (Viagen Biotech) with 1.0 mg/mL freshly prepared proteinase K (Meridian Bioscience) and incubation in a hybridization oven for 3 hours at 55° C. with rotation. The cell lysate was heat-inactivated for 45 minutes at 85° C. and stored at −20° C. prior to assaying.

Clonal HEK293 Cell Lines. Twin prime editing was used to generate single cell clones containing an att site at the adeno-associated virus integration site 1 (AAVS1) locus or Reverse Orientation Splice Acceptor 26 (ROSA26) locus. HEK293 cells were transfected with 450 ng PE5max and 150 ng of each epegRNA plasmid (a and b) in 24-well plates. Cells were serially diluted and visually confirmed to have a single colony per well. The presence of the att site was confirmed by droplet digital PCR. Transfections with clonal lines containing a preinserted att site used 360 ng of helper and 240 ng of donor. After 3 days, cells were lysed for analysis by droplet digital PCR.

Simultaneous Twin Prime Editing and Integration Assays in HEK293, HEK293T, HeLa, K562, and Primary Hepatocyte Cell Lines. HEK293, HEK293T, and HeLa cells (1×10⁵) were seeded in 24-well plates. The next day, wells were transfected with 300 ng of PE5max, 30 ng of each epegRNA, 120 ng of helper (integrase), and 120 ng of donor. K562 cells (4×10⁶) were seeded in 6-well plates. After 3 days, cells were lysed for analysis. All donor plasmids expressed DsRed. To reduce the influence of transfection efficiency, HEK293T, K562, and HeLa cells were sorted for DsRed to ensure uptake of the DNA.

Sample Preparation for Amplicon Sequencing. HEK293 cells were transfected with 600 ng of PE5max and 60 ng of each epegRNA plasmid (a and b). After 3 days, cells were lysed by incubating the pellets with 90 μL of Direct Lysis buffer supplemented with freshly prepared 1.0 mg/mL Proteinase K (Viagen Biotech) and incubation for 3 hours at 55° C. with rotation. The cell lysate was heat-inactivated for 45 minutes at 85° C. To determine editing outcomes using amplicon sequencing, a first round of PCR (PCR1) was performed using KOD One™ Blue polymerase (Toyobo), 0.3 μM of locus-specific barcoded primers containing a Nextera™ P5- and P7-tag (Table 4) and 1 μL of cell lysate in a total volume of 20 μL. An 8 bp barcode sequence was inserted between the Nextera™ tag sequence and the annealing sequence of the primers. PCR cycle conditions were 98° C. for 2 min followed by 30 cycles of 98° C. for 10 sec, 60° C. for 5 sec, and 68° C. for 5 sec with a final extension at 68° C. for 30 sec. Products from PCR1 were separated on a 2% gel in TAE (Tris-acetate-EDTA) buffer and fragments were extracted using the Zymo™ Gel DNA recovery Kit (Zymo). Fluted products were pooled in equimolar amounts and were submitted to the UC Davis DNA Technologies and Expression Analysis Core lab for a second round of PCR (PCR2) and amplicon sequencing.

TABLE 4

| | | SEQ ID |
|---|---|---|
| Primer name | Sequence | NO: |
| NGS-AAV-1F | TCGTCGGCAGCGTCAGATGTGTATAAGAGACA | 17 |
| | GGATGACCTaactgcttctcctcttgggaag | |
| NGS-AAV-2F | TCGTCGGCAGCGTCAGATGTGTATAAGAGACA | 18 |
| | GCTGTCTGTaactgcttctcctcttgggaag | |
| NGS-AAV-3F | TCGTCGGCAGCGTCAGATGTGTATAAGAGACA | 19 |
| | GCACACAGTaactgcttctcctcttgggaag | |
| NGS-AAV-4F | TCGTCGGCAGCGTCAGATGTGTATAAGAGACA | 20 |
| | GAACCGGTTaactgcttctcctcttgggaag | |
| NGS-AAV-5F | TCGTCGGCAGCGTCAGATGTGTATAAGAGACA | 21 |
| | GTCTCTCAGaactgcttctcctcttgggaag | |
| NGS-AAV-6F | TCGTCGGCAGCGTCAGATGTGTATAAGAGACA | 22 |
| | GGAGTCACTaactgcttctcctcttgggaag | |
| NGS-AAV-25F | TCGTCGGCAGCGTCAGATGTGTATAAGAGACA | 23 |
| | GAGACTGAGaactgcttctcctcttgggaag | |
| NGS-AAV-26F | TCGTCGGCAGCGTCAGATGTGTATAAGAGACA | 24 |
| | GGGAATACGaactgcttctcctcttgggaag | |
| NGS-AAV-27F | TCGTCGGCAGCGTCAGATGTGTATAAGAGACA | 25 |
| | GAGACCTGTaactgcttctcctcttgggaag | |
| NGS-AAV-28F | TCGTCGGCAGCGTCAGATGTGTATAAGAGACA | 26 |
| | GTAGGCGTTaactgcttctcctcttgggaag | |
| NGS-AAV-29F | TCGTCGGCAGCGTCAGATGTGTATAAGAGACA | 27 |
| | GTGACGACTaactgcttctcctcttgggaag | |
| NGS-AAV-7F | TCGTCGGCAGCGTCAGATGTGTATAAGAGACA | 28 |
| | GAGAGACTGaactgcttctcctcttgggaag | |
| NGS-AAV-8F | TCGTCGGCAGCGTCAGATGTGTATAAGAGACA | 29 |
| | GATATGCCGaactgcttctcctcttgggaag | |
| NGS-AAV-9F | TCGTCGGCAGCGTCAGATGTGTATAAGAGACA | 30 |
| | GCCTACGAAcactaaggcaattggggtgc | |
| NGS-AAV-10F | TCGTCGGCAGCGTCAGATGTGTATAAGAGACA | 31 |
| | GCGTAGGAAcactaaggcaattggggtgc | |
| NGS-AAV-11F | TCGTCGGCAGCGTCAGATGTGTATAAGAGACA | 32 |
| | GCTCTGACTgccaggacggggctg | |
| NGS-AAV-12F | TCGTCGGCAGCGTCAGATGTGTATAAGAGACA | 33 |
| | GGTCATCAGgccaggacggggctg | |
| NGS-Xq22-1F | TCGTCGGCAGCGTCAGATGTGTATAAGAGACA | 37 |
| | GTCACTCTGaaggagaatgacagacaatagtatatatgaaat | |
| NGS-Xq22-2F | TCGTCGGCAGCGTCAGATGTGTATAAGAGACA | 35 |
| | GCTGACAGTaaggagaatgacagacaatagtatatatgaaat | |
| NGS-Xq22-3F | TCGTCGGCAGCGTCAGATGTGTATAAGAGACA | 36 |
| | GAGTGACAGaaggagaatgacagacaatagtatatatgaaat | |
| NGS-Xq22-4F | TCGTCGGCAGCGTCAGATGTGTATAAGAGACA | 37 |
| | GACCTACCTaaggagaatgacagacaatagtatatatgaaat | |
| NGS-Xq22-5F | TCGTCGGCAGCGTCAGATGTGTATAAGAGACA | 38 |
| | GCATGTGGTaaggagaatgacagacaatagtatatatgaaat | |
| NGS-Xq22-6F | TCGTCGGCAGCGTCAGATGTGTATAAGAGACA | 39 |
| | GTTCGTAGGaaggagaatgacagacaatagtatatatgaaat | |
| NGS-Xq22-7F | TCGTCGGCAGCGTCAGATGTGTATAAGAGACA | 40 |
| | GGTCTTGAGaaggagaatgacagacaatagtatatatgaaat | |
| NGS-Xq22-8F | TCGTCGGCAGCGTCAGATGTGTATAAGAGACA | 41 |
| | GACAGAGTGaaggagaatgacagacaatagtatatatgaaat | |
| NGS-ROSA-1F | TCGTCGGCAGCGTCAGATGTGTATAAGAGACA | 42 |
| | GTTCGAACGgtgaatgactaagctccatttccc | |
| NGS-ROSA-2F | TCGTCGGCAGCGTCAGATGTGTATAAGAGACA | 43 |
| | GGATGAGGTgtgaatgactaagctccatttccc | |
| NGS-ROSA-3F | TCGTCGGCAGCGTCAGATGTGTATAAGAGACA | 44 |
| | GCCTACGTTgtgaatgactaagctccatttccc | |
| NGS-ROSA-4F | TCGTCGGCAGCGTCAGATGTGTATAAGAGACA | 45 |
| | GGAGTTGAGgtgaatgactaagctccatttccc | |
| NGS-ROSA-1F_2 | TCGTCGGCAGCGTCAGATGTGTATAAGAGACA | 46 |
| | GTTCGAACGctccatttccctacccca | |
| NGS-ROSA-2F_2 | TCGTCGGCAGCGTCAGATGTGTATAAGAGACA | 47 |
| | GGATGAGGTctccatttccctacccca | |
| NGS-ROSA-3F_2 | TCGTCGGCAGCGTCAGATGTGTATAAGAGACA | 48 |
| | GCCTACGTTctccatttccctacccca | |
| NGS-ROSA-4F_2 | TCGTCGGCAGCGTCAGATGTGTATAAGAGACA | 49 |
| | GGAGTTGAGctccatttccctacccca | |
| NGS-ROSA-5F | TCGTCGGCAGCGTCAGATGTGTATAAGAGACA | 50 |
| | GGGTTCGATgggagggaagcactggttt | |
| NGS-ROSA-6F | TCGTCGGCAGCGTCAGATGTGTATAAGAGACA | 51 |
| | GATCGTTGGgggagggaagcactggttt | |
| NGS-ROSA-7F | TCGTCGGCAGCGTCAGATGTGTATAAGAGACA | 52 |
| | GAGACTCTGgagagggagaaagctagtgctat | |
| NGS-ROSA-8F | TCGTCGGCAGCGTCAGATGTGTATAAGAGACA | 53 |
| | GCAGTACTGgagagggagaaagctagtgctat | |
| NGS-AAV-13R | GTCTCGTGGGCTCGGAGATGTGTATAAGAGAC | 54 |
| | AGCATCCAAGcccccatttcctggagcc | |
| NGS-AAV-14R | GTCTCGTGGGCTCGGAGATGTGTATAAGAGAC | 55 |
| | AGCGTTGGATcccccatttcctggagcc | |
| NGS-AAV-15R | GTCTCGTGGGCTCGGAGATGTGTATAAGAGAC | 56 |
| | AGGATCTGGTcccccatttcctggagcc | |
| NGS-AAV-16R | GTCTCGTGGGCTCGGAGATGTGTATAAGAGAC | 57 |
| | AGGTTCCTTGcccccatttcctggagcc | |
| NGS-AAV-17R | GTCTCGTGGGCTCGGAGATGTGTATAAGAGAC | 58 |
| | AGCTAGTGGTcccccatttcctggagcc | |
| NGS-AAV-18R | GTCTCGTGGGCTCGGAGATGTGTATAAGAGAC | 59 |
| | AGCAGTCAGTcccccatttcctggagcc | |
| NGS-AAV-30R | GTCTCGTGGGCTCGGAGATGTGTATAAGAGAC | 60 |
| | AGGTGTACTGcccccatttcctggagcc | |
| NGS-AAV-31R | GTCTCGTGGGCTCGGAGATGTGTATAAGAGAC | 61 |
| | AGGTTGTCCTcccccatttcctggagcc | |
| NGS-AAV-32R | GTCTCGTGGGCTCGGAGATGTGTATAAGAGAC | 62 |
| | AGACCATGGTcccccatttcctggagcc | |
| NGS-AAV-33R | GTCTCGTGGGCTCGGAGATGTGTATAAGAGAC | 63 |
| | AGCACAACTGcccccatttcctggagcc | |
| NGS-AAV-1F | TCGTCGGCAGCGTCAGATGTGTATAAGAGACA | 64 |
| | GGATGACCTaactgcttctcctcttgggaag | |
| NGS-AAV-2F | TCGTCGGCAGCGTCAGATGTGTATAAGAGACA | 65 |
| | GCTGTCTGTaactgcttctcctcttgggaag | |
| NGS-AAV-3F | TCGTCGGCAGCGTCAGATGTGTATAAGAGACA | 66 |
| | GCACACAGTaactgcttctcctcttgggaag | |
| NGS-AAV-4F | TCGTCGGCAGCGTCAGATGTGTATAAGAGACA | 67 |
| | GAACCGGTTaactgcttctcctcttgggaag | |
| NGS-AAV-5F | TCGTCGGCAGCGTCAGATGTGTATAAGAGACA | 68 |
| | GTCTCTCAGaactgcttctcctcttgggaag | |
| NGS-AAV-6F | TCGTCGGCAGCGTCAGATGTGTATAAGAGACA | 69 |
| | GGAGTCACTaactgcttctcctcttgggaag | |
| NGS-AAV-25F | TCGTCGGCAGCGTCAGATGTGTATAAGAGACA | 70 |
| | GAGACTGAGaactgcttctcctcttgggaag | |
| NGS-AAV-26F | TCGTCGGCAGCGTCAGATGTGTATAAGAGACA | 71 |
| | GGGAATACGaactgcttctcctcttgggaag | |
| NGS-AAV-27F | TCGTCGGCAGCGTCAGATGTGTATAAGAGACA | 72 |
| | GAGACCTGTaactgcttctcctcttgggaag | |
| NGS-AAV-28F | TCGTCGGCAGCGTCAGATGTGTATAAGAGACA | 73 |
| | GTAGGCGTTaactgcttctcctcttgggaag | |
| NGS-AAV-29F | TCGTCGGCAGCGTCAGATGTGTATAAGAGACA | 74 |
| | GTGACGACTaactgcttctcctcttgggaag | |
| NGS-AAV-7F | TCGTCGGCAGCGTCAGATGTGTATAAGAGACA | 75 |
| | GAGAGACTGaactgcttctcctcttgggaag | |
| NGS-AAV-8F | TCGTCGGCAGCGTCAGATGTGTATAAGAGACA | 76 |
| | GATATGCCGaactgcttctcctcttgggaag | |
| NGS-AAV-9F | TCGTCGGCAGCGTCAGATGTGTATAAGAGACA | 77 |
| | GCCTACGAAcactaaggcaattggggtgc | |
| NGS-AAV-10F | TCGTCGGCAGCGTCAGATGTGTATAAGAGACA | 78 |
| | GCGTAGGAAcactaaggcaattggggtgc | |
| NGS-AAV-11F | TCGTCGGCAGCGTCAGATGTGTATAAGAGACA | 79 |
| | GCTCTGACTgccaggacggggctg | |
| NGS-AAV-12F | TCGTCGGCAGCGTCAGATGTGTATAAGAGACA | 80 |
| | GGTCATCAGgccaggacggggctg | |
| NGS-Xq22-1F | TCGTCGGCAGCGTCAGATGTGTATAAGAGACA | 81 |
| | GTCACTCTGaaggagaatgacagacaatagtatatatgaaat | |
| NGS-Xq22-2F | TCGTCGGCAGCGTCAGATGTGTATAAGAGACA | 82 |
| | GCTGACAGTaaggagaatgacagacaatagtatatatgaaat | |
| NGS-Xq22-3F | TCGTCGGCAGCGTCAGATGTGTATAAGAGACA | 83 |
| | GAGTGACAGaaggagaatgacagacaatagtatatatgaaat | |
| NGS-Xq22-4F | TCGTCGGCAGCGTCAGATGTGTATAAGAGACA | 84 |
| | GACCTACCTaaggagaatgacagacaatagtatatatgaaat | |
| NGS-Xq22-5F | TCGTCGGCAGCGTCAGATGTGTATAAGAGACA | 85 |
| | GCATGTGGTaaggagaatgacagacaatagtatatatgaaat | |
| NGS-Xq22-6F | TCGTCGGCAGCGTCAGATGTGTATAAGAGACA | 86 |
| | GTTCGTAGGaaggagaatgacagacaatagtatatatgaaat | |
| NGS-Xq22-7F | TCGTCGGCAGCGTCAGATGTGTATAAGAGACA | 87 |
| | GGTCTTGAGaaggagaatgacagacaatagtatatatgaaat | |
| NGS-Xq22-8F | TCGTCGGCAGCGTCAGATGTGTATAAGAGACA | 88 |
| | GACAGAGTGaaggagaatgacagacaatagtatatatgaaat | |
| NGS-ROSA-1F | TCGTCGGCAGCGTCAGATGTGTATAAGAGACA | 89 |
| | GTTCGAACGgtgaatgactaagctccatttccc | |
| NGS-ROSA-2F | TCGTCGGCAGCGTCAGATGTGTATAAGAGACA | 90 |
| | GGATGAGGTgtgaatgactaagctccatttccc | |
| NGS-ROSA-3F | TCGTCGGCAGCGTCAGATGTGTATAAGAGACA | 91 |
| | GCCTACGTTgtgaatgactaagctccatttccc | |
| NGS-ROSA-4F | TCGTCGGCAGCGTCAGATGTGTATAAGAGACA | 92 |
| | GGAGTTGAGgtgaatgactaagctccatttccc | |
| NGS-ROSA-1F_2 | TCGTCGGCAGCGTCAGATGTGTATAAGAGACA | 93 |
| | GTTCGAACGctccatttccctacccca | |
| NGS-ROSA-2F_2 | TCGTCGGCAGCGTCAGATGTGTATAAGAGACA | 94 |
| | GGATGAGGTctccatttccctacccca | |
| NGS-ROSA-3F_2 | TCGTCGGCAGCGTCAGATGTGTATAAGAGACA | 95 |
| | GCCTACGTTctccatttccctacccca | |
| NGS-ROSA-4F_2 | TCGTCGGCAGCGTCAGATGTGTATAAGAGACA | 96 |
| | GGAGTTGAGctccatttccctacccca | |
| NGS-ROSA-5F | TCGTCGGCAGCGTCAGATGTGTATAAGAGACA | 97 |
| | GGGTTCGATgggagggaagcactggttt | |
| NGS-ROSA-6F | TCGTCGGCAGCGTCAGATGTGTATAAGAGACA | 98 |
| | GATCGTTGGgggagggaagcactggttt | |
| NGS-ROSA-7F | TCGTCGGCAGCGTCAGATGTGTATAAGAGACA | 99 |
| | GAGACTCTGgagagggagaaagctagtgctat | |
| NGS-ROSA-8F | TCGTCGGCAGCGTCAGATGTGTATAAGAGACA | 100 |
| | GCAGTACTGgagagggagaaagctagtgctat | |
| NGS-AAV-13R | GTCTCGTGGGCTCGGAGATGTGTATAAGAGAC | 101 |
| | AGCATCCAAGcccccatttcctggagcc | |
| NGS-AAV-14R | GTCTCGTGGGCTCGGAGATGTGTATAAGAGAC | 102 |
| | AGCGTTGGATcccccatttcctggagcc | |
| NGS-AAV-15R | GTCTCGTGGGCTCGGAGATGTGTATAAGAGAC | 103 |
| | AGGATCTGGTcccccatttcctggagcc | |
| NGS-AAV-16R | GTCTCGTGGGCTCGGAGATGTGTATAAGAGAC | 104 |
| | AGGTTCCTTGcccccatttcctggagcc | |
| NGS-AAV-17R | GTCTCGTGGGCTCGGAGATGTGTATAAGAGAC | 105 |
| | AGCTAGTGGTcccccatttcctggagcc | |
| NGS-AAV-18R | GTCTCGTGGGCTCGGAGATGTGTATAAGAGAC | 106 |
| | AGCAGTCAGTcccccatttcctggagcc | |
| NGS-AAV-30R | GTCTCGTGGGCTCGGAGATGTGTATAAGAGAC | 107 |
| | AGGTGTACTGcccccatttcctggagcc | |
| NGS-AAV-31R | GTCTCGTGGGCTCGGAGATGTGTATAAGAGAC | 108 |
| | AGGTTGTCCTcccccatttcctggagcc | |
| NGS-AAV-32R | GTCTCGTGGGCTCGGAGATGTGTATAAGAGAC | 109 |
| | AGACCATGGTcccccatttcctggagcc | |
| NGS-AAV-33R | GTCTCGTGGGCTCGGAGATGTGTATAAGAGAC | 110 |
| | AGCACAACTGcccccatttcctggagcc | |
| NGS-AAV-34R | GTCTCGTGGGCTCGGAGATGTGTATAAGAGAC | 111 |
| | AGCTCTCTCTcccccatttcctggagcc | |
| NGS-AAV-19R | GTCTCGTGGGCTCGGAGATGTGTATAAGAGAC | 112 |
| | AGTAGGAACGcccccatttcctggagcc | |
| NGS-AAV-20R | GTCTCGTGGGCTCGGAGATGTGTATAAGAGAC | 113 |
| | AGTCTCCAGTcccccatttcctggagcc | |
| NGS-AAV-21R | GTCTCGTGGGCTCGGAGATGTGTATAAGAGAC | 114 |
| | AGAGTCGACTgcctagttttagcactgaaaccc | |
| NGS-AAV-22R | GTCTCGTGGGCTCGGAGATGTGTATAAGAGAC | 115 |
| | AGAACGTAGGgcctagttttagcactgaaaccc | |
| NGS-AAV-23R | GTCTCGTGGGCTCGGAGATGTGTATAAGAGAC | 116 |
| | AGACTGGACTggccaccctgcgctac | |
| NGS-AAV-24R | GTCTCGTGGGCTCGGAGATGTGTATAAGAGAC | 117 |
| | AGACTCACAGggccaccctgcgctac | |
| NGS-Xq22-9R | GTCTCGTGGGCTCGGAGATGTGTATAAGAGAC | 118 |
| | AGCTACGTAGcccaggttacatttggttgatatgtc | |
| NGS-Xq22-10R | GTCTCGTGGGCTCGGAGATGTGTATAAGAGAC | 119 |
| | AGTTCCGGTTcccaggttacatttggttgatatgtc | |
| NGS-Xq22-11R | GTCTCGTGGGCTCGGAGATGTGTATAAGAGAC | 120 |
| | AGGACATGAGcaggtattttaaatggtccccagg | |
| NGS-Xq22-12R | GTCTCGTGGGCTCGGAGATGTGTATAAGAGAC | 121 |
| | AGCAACCATGcaggtattttaaatggtccccagg | |
| NGS-Xq22-13R | GTCTCGTGGGCTCGGAGATGTGTATAAGAGAC | 122 |
| | AGACACCTCTcaggtattttaaatggtccccagg | |
| NGS-Xq22-14R | GTCTCGTGGGCTCGGAGATGTGTATAAGAGAC | 123 |
| | AGCTCAACAGcaggtattttaaatggtccccagg | |
| NGS-Xq22-15R | GTCTCGTGGGCTCGGAGATGTGTATAAGAGAC | 124 |
| | AGACGTTGGTcaggtattttaaatggtccccagg | |
| NGS-Xq22-16R | GTCTCGTGGGCTCGGAGATGTGTATAAGAGAC | 125 |
| | AGAACGATGGcaggtattttaaatggtccccagg | |
| NGS-ROSA-9R | GTCTCGTGGGCTCGGAGATGTGTATAAGAGAC | 126 |
| | AGACTGTGTGagaagaggtcagaaagccagtc | |
| NGS-ROSA-10R | GTCTCGTGGGCTCGGAGATGTGTATAAGAGAC | 127 |
| | AGTGGTGAAGagaagaggtcagaaagccagtc | |
| NGS-ROSA-11R | GTCTCGTGGGCTCGGAGATGTGTATAAGAGAC | 128 |
| | AGCTTCTCCTagaagaggtcagaaagccagtc | |
| NGS-ROSA-12R | GTCTCGTGGGCTCGGAGATGTGTATAAGAGAC | 129 |
| | AGGAGTGTCTagaagaggtcagaaagccagtc | |
| NGS-R_2OSA- | GTCTCGTGGGCTCGGAGATGTGTATAAGAGAC | 130 |
| 9R_2 | AGACTGTGTGgtcagaaagccagtcgcg | |
| NGS-R_20SA- | GTCTCGTGGGCTCGGAGATGTGTATAAGAGAC | 131 |
| 10R_2 | AGTGGTGAAGgtcagaaagccagtcgcg | |
| NGS-R_2OSA- | GTCTCGTGGGCTCGGAGATGTGTATAAGAGAC | 132 |
| 11R 2 | AGCTTCTCCTgtcagaaagccagtcgcg | |
| NGS-R_2OSA- | GTCTCGTGGGCTCGGAGATGTGTATAAGAGAC | 133 |
| 12R_2 | AGGAGTGTCTgtcagaaagccagtcgcg | |
| NGS-ROSA-13R | GTCTCGTGGGCTCGGAGATGTGTATAAGAGAC | 134 |
| | AGTCTCAGAGggttattgtaataagggtggggtagg | |
| NGS-ROSA-14R | GTCTCGTGGGCTCGGAGATGTGTATAAGAGAC | 135 |
| | AGCAGTTCAGggttattgtaataagggtggggtagg | |
| NGS-ROSA-15R | GTCTCGTGGGCTCGGAGATGTGTATAAGAGAC | 136 |
| | AGAGTGCACTcctgagttttataccatttgagaccc | |
| NGS-ROSA-16R | GTCTCGTGGGCTCGGAGATGTGTATAAGAGAC | 137 |
| | AGACCTAGGTcctgagttttataccatttgagaccc | |
| NGS-ROSA-1F | TCGTCGGCAGCGTCAGATGTGTATAAGAGACA | 138 |
| | GTTCGAACGgtgaatgactaagctccatttccc | |
| NGS-ROSA-2F | TCGTCGGCAGCGTCAGATGTGTATAAGAGACA | 139 |
| | GGATGAGGTgtgaatgactaagctccatttccc | |
| NGS-ROSA-3F | TCGTCGGCAGCGTCAGATGTGTATAAGAGACA | 140 |
| | GCCTACGTTgtgaatgactaagctccatttccc | |
| NGS-ROSA-4F | TCGTCGGCAGCGTCAGATGTGTATAAGAGACA | 141 |
| | GGAGTTGAGgtgaatgactaagctccatttccc | |
| NGS-ROSA-1F_2 | TCGTCGGCAGCGTCAGATGTGTATAAGAGACA | 142 |
| | GTTCGAACGctccatttccctacccca | |
| NGS-ROSA-2F_2 | TCGTCGGCAGCGTCAGATGTGTATAAGAGACA | 143 |
| | GGATGAGGTctccatttccctacccca | |
| NGS-ROSA-3F_2 | TCGTCGGCAGCGTCAGATGTGTATAAGAGACA | 144 |
| | GCCTACGTTctccatttccctacccca | |
| NGS-ROSA-4F_2 | TCGTCGGCAGCGTCAGATGTGTATAAGAGACA | 145 |
| | GGAGTTGAGctccatttccctacccca | |
| NGS-ROSA-9R | GTCTCGTGGGCTCGGAGATGTGTATAAGAGAC | 146 |
| | AGACTGTGTGagaagaggtcagaaagccagtc | |
| NGS-ROSA-10R | GTCTCGTGGGCTCGGAGATGTGTATAAGAGAC | 147 |
| | AGTGGTGAAGagaagaggtcagaaagccagtc | |
| NGS-ROSA-11R | GTCTCGTGGGCTCGGAGATGTGTATAAGAGAC | 148 |
| | AGCTTCTCCTagaagaggtcagaaagccagtc | |
| NGS-ROSA-12R | GTCTCGTGGGCTCGGAGATGTGTATAAGAGAC | 149 |
| | AGGAGTGTCTagaagaggtcagaaagccagtc | |
| NGS-R_2OSA- | GTCTCGTGGGCTCGGAGATGTGTATAAGAGAC | 150 |
| 9R_2 | AGACTGTGTGgtcagaaagccagtcgcg | |
| NGS-R 2OSA- | GTCTCGTGGGCTCGGAGATGTGTATAAGAGAC | 151 |
| 10R_2 | AGTGGTGAAGgtcagaaagccagtcgcg | |
| NGS-R_2OSA- | GTCTCGTGGGCTCGGAGATGTGTATAAGAGAC | 152 |
| 11R_2 | AGCTTCTCCTgtcagaaagccagtcgcg | |
| NGS-R_2OSA- | GTCTCGTGGGCTCGGAGATGTGTATAAGAGAC | 153 |
| 12R 2 | AGGAGTGTCTgtcagaaagccagtcgcg | |
| SG Rosa 1a + 1b | gtgaatgactaagctccatttccc | 154 |
| For | | |
| SG Rosa 1a + 1b | agaagaggtcagaaagccagtc | 155 |
| rev | | |
| SG Rosa 1a + 1b | ctccatttccctacccca | 156 |
| For 2 | | |
| SG Rosa 1a + 1b | gtcagaaagccagtcgcg | 157 |
| rev2 | | |
| SG Rosa 1a + 1b | ccctaccccacccttattaca | 158 |
| For 3 | | |
| SG Rosa 1a + 1b | gaggtcagaaagccagtcgc | 159 |
| rev 3 | | |

Analysis of Amplicon Sequencing Data. Amplicon sequencing results were first demultiplexed using Ultraplex (Wilkins et al. (2021) Wellcome Open Res. 6:141) based on barcodes allowing one barcode mismatch. The barcode sequences were trimmed, and low-quality reads were discarded using a q-score of 20 (default is 30). To sort the forward and reverse reads in the same order, the sequences were re-paired with the repair function in BBtools. Editing and indel frequencies were determined as previously described (Doman et al. (2022) Nat. Protoc. 17:2431-2468). Briefly, CRISPResso2 was run in HDR mode, discard indel reads flag was on and quantification window coordinates were set to 10 base pairs upstream of each nick site. Unedited template and PE sequences were input. Data from the “CRISPResso_quantification_of_editing_frequency” file was used to determine editing frequency by dividing “Read_aligned” (in the HDR tab) by “Reads_aligned_all_amplicons”. The indel frequency was determined by dividing total discarded reads (Reference+HDR) by “Reads_aligned_all_amplicons”. Examples of prime editing sequences as well as primers are provided in Table 5.

TABLE 5

| Target | Sequence | SEQ ID NO: |
|---|---|---|
| AAV 1a + 1b (Un- | CACTAAGGCAATTGGGGTGCAGGAATGGGGGCAGGG | 160 |
| edited) | TACCAGCCTCACCAAGTGGTTGATAAACCCACGTGG | |
| | GGTACCCTAAGAACTTGGGAACAGCCACAGCAGGGG | |
| | GGCGATGCTTGGGGACCTGCCTGGAGAAGGATGCAG | |
| | GACGAGAAACACAGCCCCAGGTGGAGAAACTGGCC | |
| | GGGAATCAAGAGTCACCCAGAGACAGTGACCAACCA | |
| | TCCCTGTTTTCCTAGGACTGAGGGTTTCAGTGCTAAA | |
| | ACTAGGC | |
| AAV 1a + 1b | CACTAAGGCAATTGGGGTGCAGGAATGGGGGCAGGG | 161 |
| attB35 | TACCAGCCTCACCAAGTGGTTGATAAACCCGTGCCA | |
| | GGGCGTGCCCTTGGGCTCCCCGGGCGCGTGTGGAGA | |
| | AACTGGCCGGGAATCAAGAGTCACCCAGAGACAGTG | |
| | ACCAACCATCCCTGTTTTCCTAGGACTGAGGGTTTCA | |
| | GTGCTAAAACTAGGC | |
| AAV 1a + 1b | CACTAAGGCAATTGGGGTGCAGGAATGGGGGCAGGG | 162 |
| attP41 | TACCAGCCTCACCAAGTGGTTGATAAACCCGCCCCC | |
| | AACTGAGAGAACTCAAAGGTTACCCCAGTTGGGGCG | |
| | TGGAGAAACTGGCCGGGAATCAAGAGTCACCCAGAG | |
| | ACAGTGACCAACCATCCCTGTTTTCCTAGGACTGAGG | |
| | GTTTCAGTGCTAAAACTAGGC | |
| AAV 2a + 2b (Un- | GCCAGGACGGGGCTGGCTACTGGCCTTATCTCACAG | 163 |
| edited) | GTAAAACTGACGCACGGAGGAACAATATAAATTGGG | |
| | GACTAGAAAGGTGAAGAGCCAAAGTTAGAACTCAGG | |
| | ACCAACTTATTCTGATTTTGTTTTTCCAAACTGCTTCT | |
| | CCTCTTGGGAAGTGTAAGGAAGCTGCAGCACCAGGA | |
| | TCAGTGAAACGCACCAGACAGCCGCGTCAGAGCAGC | |
| | TCAGGTTCTGGGAGAGGGTAGCGCAGGGTGGCC | |
| AAV 2a + 2b | GCCAGGACGGGGCTGGCTACTGGCCTTATCTCACAG | 164 |
| attB35 | GTAAAACTGACGCAGTGCCAGGGCGTGCCCTTGGGC | |
| | TCCCCGGGCGCGTGGGAAGTGTAAGGAAGCTGCAGC | |
| | ACCAGGATCAGTGAAACGCACCAGACAGCCGCGTCA | |
| | GAGCAGCTCAGGTTCTGGGAGAGGGTAGCGCAGGGT | |
| | GGCC | |
| AAV 2a + 2b | GCCAGGACGGGGCTGGCTACTGGCCTTATCTCACAG | 165 |
| attP41 | GTAAAACTGACGCAGCCCCCAACTGAGAGAACTCAA | |
| | AGGTTACCCCAGTTGGGGGGGGAAGTGTAAGGAAGC | |
| | TGCAGCACCAGGATCAGTGAAACGCACCAGACAGCC | |
| | GCGTCAGAGCAGCTCAGGTTCTGGGAGAGGGTAGCG | |
| | CAGGGTGGCC | |
| AAV 3a + 3b (Un- | AACTGCTTCTCCTCTTGGGAAGTGTAAGGAAGCTGCA | 166 |
| edited) | GCACCAGGATCAGTGAAACGCACCAGACAGCCGCGT | |
| | CAGAGCAGCTCAGGTTCTGGGAGAGGGTAGCGCAGG | |
| | GTGGCCACTGAGAACCGGGCAGGTCACGCATCCCCC | |
| | CCTTCCCTCCCACCCCCTGCCAAGCTCTCCCTCCCAG | |
| | GATCCTCTCTGGCTCCATCGTAAGCAAACCTTAGAGG | |
| | TTCTGGCAAGGAGAGAGATGGCTCCAGGAAATGGGGG | |
| AAV 3a + 3b (Liu | AACTGCTTCTCCTCTTGGGAAGTGTAAGGAAGCTGCA | 167 |
| lab) attP | GCACCAGGATCAGTGAAACGCACCAGGTTTGTCTGG | |
| | TCAACCACCGCGGTCTCAGTGGTGTACGGTACAAAC | |
| | CTCCTCTCTGGCTCCATCGTAAGCAAACCTTAGAGGT | |
| | TCTGGCAAGGAGAGAGATGGCTCCAGGAAATGGGGG | |
| AAV 3a + 3b | AACTGCTTCTCCTCTTGGGAAGTGTAAGGAAGCTGCA | 168 |
| attB35 | GCACCAGGATCAGTGAAACGCACCGTGCCAGGGCGT | |
| | GCCCTTGGGCTCCCCGGGCGCGTTCCTCTCTGGCTCC | |
| | ATCGTAAGCAAACCTTAGAGGTTCTGGCAAGGAGAG | |
| | AGATGGCTCCAGGAAATGGGGG | |
| AAV 3a + 3b | AACTGCTTCTCCTCTTGGGAAGTGTAAGGAAGCTGCA | 169 |
| attP41 | GCACCAGGATCAGTGAAACGCACCGCCCCCAACTGA | |
| | GAGAACTCAAAGGTTACCCCAGTTGGGGCTCCTCTCT | |
| | GGCTCCATCGTAAGCAAACCTTAGAGGTTCTGGCAA | |
| | GGAGAGAGATGGCTCCAGGAAATGGGGG | |
| AAV 4a + 3b (Un- | AACTGCTTCTCCTCTTGGGAAGTGTAAGGAAGCTGCA | 170 |
| edited) | GCACCAGGATCAGTGAAACGCACCAGACAGCCGCGT | |
| | CAGAGCAGCTCAGGTTCTGGGAGAGGGTAGCGCAGG | |
| | GTGGCCACTGAGAACCGGGCAGGTCACGCATCCCCC | |
| | CCTTCCCTCCCACCCCCTGCCAAGCTCTCCCTCCCAG | |
| | GATCCTCTCTGGCTCCATCGTAAGCAAACCTTAGAGG | |
| | TTCTGGCAAGGAGAGAGATGGCTCCAGGAAATGGGGG | |
| AAV 4a + 3b (Liu | AACTGCTTCTCCTCTTGGGAAGTGTAAGGAAGCTGCA | 171 |
| lab) attP | GCACCAGGATCAGTGAAACGCACCAGACAGCCGCGT | |
| | CAGAGCAGCTCAGGTTCTGGGAGGTTTGTCTGGTCA | |
| | ACCACCGCGGTCTCAGTGGTGTACGGTACAAACCTC | |
| | CTCTCTGGCTCCATCGTAAGCAAACCTTAGAGGTTCT | |
| | GGCAAGGAGAGAGATGGCTCCAGGAAATGGGGG | |
| AAV 4a + 3b | AACTGCTTCTCCTCTTGGGAAGTGTAAGGAAGCTGCA | 172 |
| attB35 | GCACCAGGATCAGTGAAACGCACCAGACAGCCGCGT | |
| | CAGAGCAGCTCAGGTTCTGGGGTGCCAGGGCGTGCC | |
| | CTTGGGCTCCCCGGGCGCGTTCCTCTCTGGCTCCATC | |
| | GTAAGCAAACCTTAGAGGTTCTGGCAAGGAGAGAGA | |
| | TGGCTCCAGGAAATGGGGG | |
| AAV 4a + 3b | AACTGCTTCTCCTCTTGGGAAGTGTAAGGAAGCTGCA | 173 |
| attP41 | GCACCAGGATCAGTGAAACGCACCAGACAGCCGCGT | |
| | CAGAGCAGCTCAGGTTCTGGGGCCCCCAACTGAGAG | |
| | AACTCAAAGGTTACCCCAGTTGGGGCTCCTCTCTGGC | |
| | TCCATCGTAAGCAAACCTTAGAGGTTCTGGCAAGGA | |
| | GAGAGATGGCTCCAGGAAATGGGGG | |
| Rosa 3a + 3b (Un- | GGGAGGGAAGCACTGGTTTCTCAAGCAAAAGCTAAA | 174 |
| edited) | ATTTTTCTATTAAGATTTAACCTGATGCTACACTTTGGT | |
| | GGTGCAGCAAGGGTCTCAAATGGTATAAAACTCAGGT | |
| | GATCATGCTTTATGTCTGTCTCTAGAAAAATGCTCCAA | |
| | AAATGATAAGTAGTGATAATCCGCAGTCTCGTTGCATA | |
| | AAATCAGCCCCAGGTGAATGACTAAGCTCCATTTCCC | |
| | TACCCCACCCTTATTACAATAACC | |
| Rosa 3a + 3b | GGGAGGGAAGCACTGGTTTCTCAAGCAAAAGCTAAA | 175 |
| attB35 | ATTTTTCTATTAAGATTTAACCTGATGCTACACTTTGGT | |
| | GGTGCAGCAAGGGTCTCAAATGGTATAAAAGTGCCA | |
| | GGGCGTGCCCTTGGGCTCCCCGGGCGCGTGTGAATG | |
| | ACTAAGCTCCATTTCCCTACCCCACCCTTATTACAATA | |
| | ACC | |
| Rosa 3a + 3b | GGGAGGGAAGCACTGGTTTCTCAAGCAAAAGCTAAA | 176 |
| attP41 | ATTTTTCTATTAAGATTTAACCTGATGCTACACTTTGGT | |
| | GGTGCAGCAAGGGTCTCAAATGGTATAAAAGCCCCCA | |
| | ACTGAGAGAACTCAAAGGTTACCCCAGTTGGGGCGT | |
| | GAATGACTAAGCTCCATTTCCCTACCCCACCCTTATTA | |
| | CAATAACC | |
| Rosa 4a + 4b (Un- | GAGAGGGAGAAAGCTAGTGCTATGTCTGAATACTAGA | 177 |
| edited) | GGAGCAAGTACAACAAATGGAAAATGGGATCAAGTA | |
| | TGAGTGAGAGTTGCTAAGATGCCTGGTAGGGATGCAA | |
| | AGGGGTAGAGAGCCTGGGGAGAGAGGGTGAGGGAG | |
| | GGAAGCACTGGTTTCTCAAGCAAAAGCTAAAATTTTT | |
| | CTATTAAGATTTAACCTGATGCTACACTTTGGTGGTGC | |
| | AGCAAGGGTCTCAAATGGTATAAAACTCAGG | |
| Rosa 4a + 4b | GAGAGGGAGAAAGCTAGTGCTATGTCTGAATACTAGA | 178 |
| attB35 | GGAGCAAGTACAACAAATGGAAAATGGGATCAAGTA | |
| | TGAGTGAGAGTTGCTAAGATGCCTGGTAGGGATGCGT | |
| | GCCAGGGCGTGCCCTTGGGCTCCCCGGGCGCGTGCTA | |
| | CACTTTGGTGGTGCAGCAAGGGTCTCAAATGGTATAA | |
| | AACTCAGG | |
| Rosa 4a + 4b | GAGAGGGAGAAAGCTAGTGCTATGTCTGAATACTAGA | 179 |
| attP41 | GGAGCAAGTACAACAAATGGAAAATGGGATCAAGTA | |
| | TGAGTGAGAGTTGCTAAGATGCCTGGTAGGGATGCGC | |
| | CCCCAACTGAGAGAACTCAAAGGTTACCCCAGTTGG | |
| | GGCGCTACACTTTGGTGGTGCAGCAAGGGTCTCAAAT | |
| | GGTATAAAACTCAGG | |
| Xq22 1a + 1b (Un- | AAGGAGAATGACAGACAATAGTATATATGAAATATTCT | 180 |
| edited) | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAATGTGGGAAA | |
| | CTTAGGAAGACACCCAGTTTCTTCAACAAAAAAATTG | |
| | CAAAAATAAAAAGGAACAAGGAGAACCTATAGATTA | |
| | AATGAGGCATTAAAGACATATCAACCAAATGTAACCT | |
| | GGG | |
| Xq22 1a + 1b | AAGGAGAATGACAGACAATAGTATATATGAAATATTCT | 181 |
| attB35 | TATCAAAAGATAAGATCTACTCTAATTAAACCTGTGCC | |
| | AGGGCGTGCCCTTGGGCTCCCCGGGCGCGTGATTAAA | |
| | TGAGGCATTAAAGACATATCAACCAAATGTAACCTGGG | |
| Xq22 1a + 1b | AAGGAGAATGACAGACAATAGTATATATGAAATATTCT | 182 |
| attP41 | TATCAAAAGATAAGATCTACTCTAATTAAACCTGCCCC | |
| | CAACTGAGAGAACTCAAAGGTTACCCCAGTTGGGGC | |
| | GATTAAATGAGGCATTAAAGACATATCAACCAAATGTA | |
| | ACCTGGG | |
| Xq22 1a + 2b (Un- | AAGGAGAATGACAGACAATAGTATATATGAAATATTCT | 183 |
| edited) | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAATGTGGGAAA | |
| | CTTAGGAAGACACCCAGTTTCTTCAACAAAAAAATTG | |
| | CAAAAATAAAAAGGAACAAGGAGAACCTATAGATTA | |
| | AATGAGGCATTAAAGACATATCAACCAAATGTAACCT | |
| | GGGGACCATTTAAAATACCTG | |
| Xq22 1a + 2b | AAGGAGAATGACAGACAATAGTATATATGAAATATTCT | 184 |
| attB35 | TATCAAAAGATAAGATCTACTCTAATTAAACCTGTGCC | |
| | AGGGCGTGCCCTTGGGCTCCCCGGGCGCGTGTAACCT | |
| | GGGGACCATTTAAAATACCTG | |
| Xq22 1a + 2b | AAGGAGAATGACAGACAATAGTATATATGAAATATTCT | 185 |
| attP41 | TATCAAAAGATAAGATCTACTCTAATTAAACCTGCCCC | |
| | CAACTGAGAGAACTCAAAGGTTACCCCAGTTGGGGC | |
| | GTAACCTGGGGACCATTTAAAATACCTG | |
| Xq22 2a + 1b (Un- | AAGGAGAATGACAGACAATAGTATATATGAAATATTCT | 186 |
| edited) | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAATGTGGGAAA | |
| | CTTAGGAAGACACCCAGTTTCTTCAACAAAAAAATTG | |
| | CAAAAATAAAAAGGAACAAGGAGAACCTATAGATTA | |
| | AATGAGGCATTAAAGACATATCAACCAAATGTAACCT | |
| | GGGGACCATTTAAAATACCTG | |
| Xq22 2a + 1b | AAGGAGAATGACAGACAATAGTATATATGAAATATTCT | 187 |
| attB35 | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAGTGCCAGGGC | |
| | GTGCCCTTGGGCTCCCCGGGCGCGTGATTAAATGAGG | |
| | CATTAAAGACATATCAACCAAATGTAACCTGGGGACC | |
| | ATTTAAAATACCTG | |
| Xq22 2a + 1b | AAGGAGAATGACAGACAATAGTATATATGAAATATTCT | 188 |
| attP41 | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAGCCCCCAACTG | |
| | AGAGAACTCAAAGGTTACCCCAGTTGGGGCGATTAA | |
| | ATGAGGCATTAAAGACATATCAACCAAATGTAACCTG | |
| | GGGACCATTTAAAATACCTG | |
| Xq22 2a + 2b (Un- | AAGGAGAATGACAGACAATAGTATATATGAAATATTCT | 189 |
| edited) | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAATGTGGGAAA | |
| | CTTAGGAAGACACCCAGTTTCTTCAACAAAAAAATTG | |
| | CAAAAATAAAAAGGAACAAGGAGAACCTATAGATTA | |
| | AATGAGGCATTAAAGACATATCAACCAAATGTAACCT | |
| | GGGGACCATTTAAAATACCTG | |
| Xq22 2a + 2b | AAGGAGAATGACAGACAATAGTATATATGAAATATTCT | 190 |
| attB35 | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAGTGCCAGGGC | |
| | GTGCCCTTGGGCTCCCCGGGCGCGTGTAACCTGGGGA | |
| | CCATTTAAAATACCTG | |
| Xq22 2a + 2b | AAGGAGAATGACAGACAATAGTATATATGAAATATTCT | 191 |
| attP41 | TATCAAAAGATAAGATCTACTCTAATTAAACCTGTAGG | |
| | GAAAAAAGGGACAAAGGAGCATGTTAAACAATATCA | |
| | TGAGAATAGAATGAGCAAACTCTAGAGCCCCCAACTG | |
| | AGAGAACTCAAAGGTTACCCCAGTTGGGGCGTAACC | |
| | TGGGGACCATTTAAAATACCTG | |

Droplet Digital PCR (ddPCR) Protocol. ddPCR was performed according to manufacturer's instructions (BioRad Laboratories). Briefly, each reaction (22 μL) contained 1.0 μL of cell lysate (1:1 dilution in water), 11 μL of 2×ddPCR Supermix for Probes (no dUTP), 10U XhoI (New England Biolabs), primers (Table 6) for target and reference at 900 nM and probes at 250 nM. The plate was sealed with PX1 PCR Plate Sealer (BioRad), vortexed thoroughly to mix, and centrifuged 1 minute at 1000 rpm prior to droplet generation using the QX200 AutoDG Droplet Generator System (BioRad). PCR was performed as follows: denaturation at 95° C. for 10 min, 45 cycles of 95° C. for 30 sec, 60° C. for 2 min and 72° C. for 2 min, followed by a final incubation at 98° C. for 10 min. Droplets were read on a QX200 Droplet Reader (BioRad) and analyzed using QX Manager 1.2 Standard Edition software (BioRad).

TABLE 6

| Primer | Sequence | SEQ ID NO: |
|---|---|---|
| ddPCR pCMV Rev | gtttgtccaaactcagcggc | 192 |
| ddPCR pCMV OPP Rev | gaaggtacgcctgcaggtac | 193 |
| ddPCR pCMV OPP Rev2 | atccagcctccggactctag | 194 |
| ddPCR attB35 Rev | ccggggagcccaaggg | 195 |
| ddPCR attP41 Rev | gagttctctcagttgggggc | 196 |
| ddPCR attP41 Rev2 | ccaactggggtaacctttgagt | 197 |
| ddPCR Rosa1a For | tccgcagtctcgttgcataa | 198 |
| ddPCR Rosa3a For | acaacaaatggaaaatgggatca | 199 |
| ddPCR Rosa3a For2 | agaacgtgaactagggagga | 200 |
| ddPCR Xq1a For | agcctctatattctaatccacttgt | 201 |
| ddPCR Xq1a For2 | ctgtgcctagcctaagcctc | 202 |
| ddPCR Xq1a For3 | tgctggaattacaggcgtga | 203 |
| ddPCR AAV3a For | gcttctcctcttgggaagtgtaa | 204 |
| ddPCR AAV2a For | aagggagttttccacacgga | 205 |
| ddPCR AAV1a For | cactaaggcaattggggtgc | 206 |
| ddPCR BxBI attB38 Rev | gtcgacgacggcggtctc | 207 |
| ddPCR BxBI attB38 Rev2 | ctccgtcgtcaggatcatcc | 208 |
| ddPCR BxBI attP48 Rev | gtacaccactgagaccgcg | 209 |
| ddPCR Pa01 attB33 Rev | ccgtgacctacatgctcgc | 210 |
| ddPCR Pa01 attB33 Rev2 | ctcgaagggcgtatgcgc | 211 |
| ddPCR Rosa1a Probe | ccccaggtgaatgactaagctccatttccctac | 212 |
| Opti Rosa1a Probe | ggggagtgagcagctgtaag | 213 |
| ddPCR Rosa3a Probe | agtgagagttgctaagatgcctggtagggatgc | 214 |
| Opti Rosa3a Probe | tgctgcaccaccaaagtgta | 215 |
| ddPCR Xq1a Probe | tagggccctgatatgggcacccaaatgtagctt | 216 |
| Opti Xq1a Probe | aacatgctcctttgtccctt | 217 |
| ddPCR AAV3a Probe | ctgcagcaccaggatcagtgaaacgcac | 218 |
| Opti AAV3a Probe | gttctcagtggccaccctg | 219 |
| ddPCR AAV2a Probe | cccctcctcaccacagccctgcca | 220 |
| Opti AAV2a Probe | ggctcttcacctttctagtccc | 221 |
| ddPCR AAV1a Probe | taccagcctcaccaagtggttgataaacccacg | 222 |
| Opti AAV1a Probe | ctcgtcctgcatccttctcc | 223 |
| ACTB For | acactgtgcccatctac | 224 |
| ACTB Rev | aatgtcacgcacgatttc | 225 |
| RPP30 For | agatttggacctgcgagcg | 226 |
| RPP30 Rev | gagcggctgtctccacaagt | 227 |
| UBE2D2 For | gtactcttgtccatctgttctctg | 228 |
| UBE2D2 Rev2 | ggccgatattcagcccttaaac | 229 |

Phage-Assisted Continuous Evolution. PACE achieves selection by linking the desired activity of the gene of interest to the production of infectious phage progeny. Expression of the phage's gene III (gIII) is proportionally related to proliferation. Using an accessory plasmid (AP), gIII expression is solely driven by the desired activity of the evolving gene. Each selection phage (SP) encodes the gene to be evolved. Phage encoding active variants of the gene produce infectious progeny, and phage encoding less-active variants fail to produce as many progeny and become outcompeted. Beneficial mutations multiply with successful phage life cycles. An inducible mutagenesis plasmid (MP) increases the mutation rate by decreasing proofreading and increasing error-prone bypass during replication.

During PACE, host cells containing the AP, XP, and MP are continually added to a fixed-volume flask (the ‘lagoon’) where they encounter phage. After infection, host cells begin expressing the integrase protein from the SP. A successfully evolved integrase, bearing beneficial mutations enhancing its activity on the attP site, aligns the gIII promoter on the AP. Activated gIII expression leads to propagation of the SP that encoded the improved integrase. These SP are again mutated during the next cycle of DNA replication, effectively generating a new library from the fittest variants of the previous library. Fresh host cells are then infected by this new library within the lagoon and the process is repeated. The proliferation of the phage scales with increasing levels of gIII expression over a range of two orders of magnitude. This establishes a competition between mutation library members resulting in the most active variants outcompeting the others.

PACE strategy and Selection Stringency. In addition to the MP for mutagenesis, host cells contained the AP and CP to enable selection for integrases with improved activity. The AP contained the attP site upstream of the C-terminus of a split gIII. The CP contained a promoter aligned with the N-terminus of gIII (the leader sequence) and the attB site. Successful crossover of the att sites on the AP and CP by an active integrase variant forms a single plasmid with a complete gIII. The resulting attL site is encoded and resides in a linker region between the leader sequence and N1 domain to prevent disruption of gene III activity. Stringency was periodically increased during each PACE experiment by decreasing promoter strength, decreasing plasmid copy number, or increasing flow rate.

Phage Preparation and Mutagenesis. The SP backbone was modified by replacing the gene VI promoter with RecA to decrease generation of recombinant wild-type M13 phage. Lagoons were infected with 10⁸ PFU of starter phage. Mutagenesis via MP6 plasmid was induced at concentrations of 100 mM arabinose. Lagoons were sampled every 1-4 hours throughout each PACE experiment.

PACE procedures were as described by Miller et al. ((2020) Nat. Protoc. 15(12):4101-4127) with modifications. In particular, the modified PACE setup included two chemostats that were maintained at volumes ranging from 300-400 mL in 2 L bottles, in which host cell cultures were changed every 2 days to insure arabinose sensitivity.

S2060 cells were transformed with AP, XP, and MP6 plasmids and cultured in Davis Rich Media (DRM) (Harvard Custom Media A and C—US Biological) supplemented with trace metals, 0.1% polysorbate 80, and appropriate antibiotics to maintain plasmids (AP—50 μg/mL Carbenicillin, CP—50 μg/mL Kanamycin, MP—25 μg/mL Chloramphenicol) in addition to 10 μg/mL Tetracycline, 10 μg/mL Flucconazole, and 10 μg/mL Amphotericin B). Host cell colonies were tested for sensitivity to arabinose before expansion to chemostats. A fixed volume 100 mL mixer in a 500 mL bottle was used to mix the two cultures before distribution to 2-6 lagoons which were generally maintained at 40 mL in a 100 mL bottle (except when extremely high flow rates were used to increase stringency) and flow rates ranged from 1-10 V/hr. L-arabinose was supplied to the lagoons at a constant rate of 0.5 μL/min (concentration was dependent on flow rate to maintain 100 mM L-arabinose in the lagoon). In experiments with 6 lagoons, a second chemostat was added along with a mixer prior to distribution to the lagoons which was maintained at 100 mL (Tables 7 and 8).

TABLE 7

| PACE | Time | | AP | CP | Flow Rate | |
|---|---|---|---|---|---|---|
| PhiC31 | 0-72 | hr | AP pUC PhiC31 attP | CP RSF1030 PhiC31 attB | 2 | V/hr |
| PACE | | | | ProB | | |
| | 72-120 | hr | AP pUC PhiC31 attP | CP RSF1030 PhiC31 attB | 2 | V/hr |
| | | | | ProA | | |
| | 120-168 | hr | AP pUC PhiC31 attP | CP RSF1030 PhiC31 attB | 2 | V/hr |
| | | | | Pro3 | | |
| | 168-192 | hr | AP pUC PhiC31 attP | CP RSF1030 PhiC31 attB | 2 | V/hr |
| | | | | Pro1 | | |
| | 192-212 | hr | AP pUC PhiC31 attP | CP RSF1030 PhiC31 attB | 3 | V/hr |
| | | | | Pro1 | | |
| Bxb1 | 0-24 | hr | AP pUC Bxb1 attP | CP RSF1030 Bxb1 attB | 2 | V/hr |
| PACE 1 | | | | ProB | | |
| | 24-48 | hr | AP pUC Bxb1 attP | CP RSF1030 Bxb1 attB | 2 | V/hr |
| | | | | ProA | | |
| | 48-72 | hr | AP pUC Bxb1 attP | CP RSF1030 Bxb1 attB Pro3 | 2 | V/hr |
| | 72-124 | hr | AP pUC Bxb1 attP | CP RSF1030 Bxb1 attB Pro1 | 2 | V/hr |
| Bxb1 | 0-24 | hr | AP pUC Bxb1 attP | CP RSF1030 Bxb1 attB Pro1 | 3 | V/hr |
| PACE 2 | 24-48 | hr | AP pUC Bxb1 attP | CP RSF1030 Bxb1 attB Pro1 | 4 | V/hr |
| | 48-72 | hr | AP pUC Bxb1 attP | CP RSF1030 Bxb1 attB Pro1 | 6 | V/hr |
| Bxb1 | 0-24 | hr | AP pUC Bxb1 attP | CP RSF1030 Bxb1 attB Pro1 | 2 | V/hr |
| PACE 3 | 24-48 | hr | AP pUC Bxb1 attP | CP RSF1030 Bxb1 attB Pro1 | 4 | V/hr |
| | 48-72 | hr | AP pUC Bxb1 attP | CP RSF1030 Bxb1 attB Pro1 | 6 | V/hr |
| | 72-96 | hr | AP pUC Bxb1 attP | CP RSF1030 Bxb1 attB Pro1 | 8 | V/hr |
| | 96-98 | hr | AP pUC Bxb1 attP | CP RSF1030 Bxb1 attB Pro1 | 10 | V/hr |
| Bxb1 | 0-48 | hr | AP R6k Bxb1 attP | CP RSF1030 Bxb1 attB Pro1 | 2 | V/hr |
| PACE 4 | 48-72 | hr | AP R6k Bxb1 attP | CP RSF1030 Bxb1 attB Pro1 | 3 | V/hr |
| | 72-96 | hr | AP R6k Bxb1 attP | CP RSF1030 Bxb1 attB Pro1 | 4 | V/hr |

TABLE 8

| | |
|---|---|
| PACE | Starting Material |

| | | |
|---|---|---|
| PhiC31 | Lagoon 1 | SP RecA PhiC31 Int605 (wild-type) |
| PACE | Lagoon 2 | SP RecA PhiC31 P2 |
| | Lagoon 3 | SP RecA PhiC31 P1 |
| | Lagoon 4 | SP RecA PhiC31 P1 |
| | Lagoon 5 | SP RecA PhiC31 P3 |
| | Lagoon 6 | SP RecA PhiC31 P3 |
| Bxb1 | Lagoon 1 | SP RecA Bxb1 (wild-type) |
| PACE 1 | Lagoon 2 | SP RecA Bxb1 (wild-type) |
| | Lagoon 3 | SP RecA Bxb1 (wild-type) |
| | Lagoon 4 | SP RecA Bxb1 (wild-type) |
| | Lagoon 5 | SP RecA Bxb1 (wild-type) |
| | Lagoon 6 | SP RecA Bxb1 (wild-type) |
| Bxb1 | Lagoon 1 | SP RecA Bxb1 PACE1 L4-100 hr |
| PACE 2 | Lagoon 2 | SP RecA Bxb1 PACE1 L6-100 hr |
| Bxb1 | Lagoon 1 | SP RecA Bxb1 L2-9 |
| PACE 3 | Lagoon 2 | SP RecA Bxb1 PACE2 pool |
| Bxb1 | Lagoon 1 | SP RecA Bxb1 (wild-type) |
| PACE 4 | Lagoon 2 | SP RecA Bxb1-c2 |
| | Lagoon 3 | SP RecA Bxb1-c3 |
| | Lagoon 4 | SP RecA Bxb1-c9 |
| | Lagoon 5 | SP RecA Bxb1-c22 |
| | Lagoon 6 | SP RecA Bxb1 L7-3 |

Phage Preparation and Mutagenesis. The SP backbone was modified by replacing the gene VI promoter with RecA to decrease generation of recombinant wild-type phage. Lagoons were infected with 10⁸ PFU of starter phage. Mutagenesis via MP6 plasmid was induced at concentrations of 100 mM arabinose. Lagoons were sampled every 1-4 hours throughout each PACE experiment.

Phage Enrichment Assay. E. coli cultures containing both AP and CP (2 mL, OD₆₀₀=0.3-0.6) were inoculated with 10⁵ PFU/mL of purified M13 phage and incubated at 37° C. at 180 rpm for 18 hours. Cultures were centrifuged at 12,000×g for 2 minutes and supernatants were collected. Phage counts were obtained by activity-independent plaque assay in S2208 cells (Badran et al. (2016) Nature 533:58-63).

Flipper Flow Cytometry Assay. Helper plasmids encoding integrase variants (100 ng) and a flipper plasmid containing ZsGreen flanked by attB and attP sites (500 ng; Table 9) were transfected into 24-well plates seeded with HEK293T cells as described above. Following 3 days of culture, cells were resuspended using cold Live Cell Imaging Solution (Thermo Fisher Scientific) and fluorescein-5-isothiocyanate (FITC) emission was assessed by flow cytometry (excitation 488 nm, emission 530 nm (BL1)) of >20,000 live, single cells per condition using an Attune NXT flow cytometer (Thermo Fisher Scientific).

TABLE 9

| Phage | Minimal | | SEQ ID |
|---|---|---|---|
| Integrase | att | Sequence | NO: |
| PhiC31¹ | attB | gtgccagggcgtgcccttgggctccccgggcgcgt | 15 |
| | attP | tgccccaactggggtaacctttgagttctctcagttgggggc | 16 |
| Bxb1² | attB | ggcttgtcgacgacggcggtctccgtcgtcaggatcat | 13 |
| | attP | ggtttgtctggtcaaccaccgcggtctcagtggtgtacggta | 14 |
| | | caaacc | |
| PaO1³ | attB | ccgtgacctacatgctcgaagggcgtatgcgcc | 230 |
| | attP | gtaacgctcttcgagaaagcagattctcatatccatcttgagt | 231 |
| | | cttctttctcgcaagacaacacgaaatagacacagtctcttc | |
| | | cctagctgtacactgagcc | |

¹Groth et al. (2009) Proc. Natl. Acad. Sci. USA 97:5995-6000;
²Ghosh et al. (2003) Mol. Cell 12(5):1101-11;
³Durrant et al. (2023) Nat. Biotechnol. 41:488-499,

Rationale for Normalization Using the Product/Reference Ratio (PRR). Two options exist for the location of the target probe: (1) The probe can be placed in the donor, with the forward primer in the donor and the reverse primer in the genome, or (2) The probe can be placed in the genome, with the forward primer in the donor and the reverse primer in the genome. An issue with the donor-located probe is that insertion at an off-target sequence positions the probe and reverse primer downstream of an unknown genomic sequence. Mispriming from the genome could generate a false signal. A second issue is that a donor-located probe cannot be used to determine the ratio of potential target sequences to reference sequences. If a greater number of target sequences exist than reference sequences, then a greater number of opportunities exist for targeting, and the final targeting efficiency will be over-reported. Accounting for this issue is especially important when assaying cell lines with abnormal numbers of chromosomes and where the reference sequence often is located on a different chromosome than the target sequence. To prevent ddPCR over- or under-reporting, the ratio of target product to reference signal was defined as the product/reference ratio (PRR). Targeting efficiencies for unknown samples were divided by the PRR to normalize for differences in available targets and references. To determine the PRR, untransfected cell lines were assayed using FAM probes designed to bind the genomic target sequence. The reverse primer is located in the genome and a second PRR optimization primer is also located in the genome and flanks the probe. The ddPCR is run with standard HEX probes designed to bind the genomic reference sequence that are flanked by genomic primers. PRR values for target sequences used herein are provided in Table 10. Templates were lysates isolated from wells of untransfected cells from the indicated cell lines (n=3). HEK293 clones were first edited by prime editing for insertion of the indicated att site. Probes were designed to bind the genomic target sequence. Primers for the HEK293 clones included the forward primer in the att site and the reverse primer in the genome. Primers for the remaining cell lines included the forward primer and reverse primer in the genome.

TABLE 10

| ref | cell line | target | ave PRR | stdev |
|---|---|---|---|---|
| RPP30 | HEK293 | ROSA 1a | 0.72 | 0.034 |
| | HEK293 | ROSA 3a | 0.76 | 0.037 |
| | HEK293 | Xq22 1a | 1.01 | 0.087 |
| | HEK293 | AAV 3a | 0.47 | 0.007 |
| | HEK293 | AAV 2a | 0.43 | 0.007 |
| | HEK293 | AAV 1a | 0.42 | 0.017 |
| UBE2D2 | HEK293 | ROSA 3a | 1.01 | 0.006 |
| | HEK293 | AAV 3a | 0.63 | 0.017 |
| | HEK293T | ROSA 3a | 1.22 | 0.011 |
| | HeLa | ROSA 3a | 0.92 | 0.008 |
| | K562 | ROSA 3a | 1.24 | 0.004 |
| | HEK293 clone | PhiC31 attP41 ROSA 3a | 0.67 | 0.004 |
| | HEK293 clone | PhiC31 attP41 AAV 3a | 0.65 | 0.004 |
| | HEK293 clone | Bxb1 attB38 ROSA 3a | 0.38 | 0.011 |

Off Target Analysis. Genomic PCR primers (Table 11) were generated to previously predicted Bxb1 pseudo sites (Anzalone et al. (2022) Nat. Biotechnol. 40:731-740; Yarnall et al. (2023) Nat. Biotechnol. 41:500-512). For each site, two primer sets were designed. The first (control) set was made to amplify the genome to verify ample template was used. The second set was made to amplify the junction of the genome and donor. Additional primers were generated to amplify successful insertion at ROSA26. Lysates from HEK293T transfections performed on two separate days were used as template for genomic PCR using the Q5 polymerase (NEB) and visualized on a 20 agarose gel.

TABLE 11

| | | SEQ |
|---|---|---|
| | | ID |
| Primer | Sequence | NO: |
| Rosa OnT Rev | ttctagagacagacataaagcatgatca | 232 |
| DL OT1 fwd | GGAAATAAGTTATCACAATGGGAAAT | 233 |
| DL OT1 rev | TCGCGATTCTTAAAAGGAGAGG | 234 |
| DL OT2 fwd | CCATTCATATTTTGAAACAAAAGG | 235 |
| DL OT2 rev | GCATTGCACTCCTACATACAACA | 236 |
| DL OT3 fwd | GCTGTGGTTATTCCCAGCTC | 237 |
| DL OT3 rev | CTGGGAACACTGGACAAAATCC | 238 |
| DL OT4 fwd | GGAAAGCTTTGACAAGTGGAA | 239 |
| DL OT4 rev | GCCTACTTGCCCTTCTTCCT | 240 |
| PASTE ch2 off For | AGGGACCTTTGCCTGTGTGAGTC | 241 |
| PASTE ch2 off Rev | cactcacacagacagaggcc | 242 |
| PASTE ch3 off For | CCAGGTGAGAGTCAGGGTAGTGTTCA | 243 |
| PASTE ch3 off Rev | gcttgcgcggcctacgt | 244 |
| PASTE ch9 off For | TCAGCTCTGTGCTGAGGCGAA | 245 |
| PASTE ch9 off Rev | GCACAACCCTGGCTGTCC | 246 |
| ddPCR pCMV Rev2 | tgaatgcaattgttgttgttaacttgt | 247 |

Factor VIII and Von Willebrand Factor. To measure expression and export of recombinant proteins, HEK293T cells were transfected with 300 ng of PE5max, 30 ng of each epegRNA plasmid, 120 ng of helper (integrase), 120 ng of donor, and 40 ng of DsRed expression plasmid (for sorting). Media was collected three days post transfection and Factor VIII protein expression was assayed using the Human Coagulation Factor VIII Total Antigen ELISA kit (Innovative Research) and Factor VIII protein activity was assayed using the Chromogenix Coamatic® Factor VIII kit (DiaPharma). Von Willebrand Factor was detected using the VWF Human ELISA Kit (Invitrogen).

### Example 2: Directed Evolution of Serine Integrases Improves Site Specific Insertion of Transgenes Using Prime Editing

Previously, directed evolution was used to improve PhiC31 integrase activity on its native att sites (Keravala et al. (2009) Mol. Ther. 17(1):112-20). It was posited that PhiC31 and its improved variants could increase targeted integrase insertion efficiency. Initially, previously characterized safe harbor loci were selected that were distant from cancer-related genes and shown to permit expression of inserted genes. These included AAVS1 (Oceguera-Yanez et al. (2016) Methods 101:43-55), Xq22.1 (Chalberg et. al. (2006) J. Mol. Biol. 357 (1):28-48), and ROSA26 (Irion et al. (2007) Nat. Biotechnol. 25:1477-1482). PE uses a fusion protein consisting of a Cas9 nickase and reverse transcriptase called PE5, as well as a prime editing guide RNA (pegRNA). The pegRNA is a modified version of a single guide RNA (sgRNA) that both directs PE5 to the target genomic sequence and encodes the desired edit. The pegRNA contains a 5′ spacer sequence that directs PE5 to nick the target sequence. The primer binding site (PBS) found on the 3′ end of the pegRNA anneals to the non-target DNA strand following the nick in the target strand. The pegRNA contains a reverse transcriptase template (RTT) that is reverse transcribed to generate a 3′ single-strand flap of DNA that encodes the inserted DNA, such as the att site. During twin PE, a second pegRNA directs the insertion of an adjacent flap that includes overlapping homologous sequence so that the two flaps form a double-stranded intermediate. Upon resolution of the intermediate, the new sequence becomes permanently inserted.

Because prime editing efficiency is generally higher for shorter inserts, pegRNA were designed for inserting the minimal PhiC31 attB site (35 bp) or attP site (41 bp). To maximize efficiency, an epegRNA containing a structured RNA motif was used to prevent degradation (Nelson et al. (2022) Nat. Biotechnol. 40 (3):402-410). In addition, an optimized cr772 scaffold (Jost et al. (2020) Nat. Biotechnol. 38(3):355-364) and PE5max which includes a dominant negative mutant of human mutL homolog 1 (MLH1) involved in DNA mismatch repair with an optimized editor architecture were used (Chen et al. (2021) Cell 184 (22):5635-5652.e29) (Tables 15-16). PE5max and two twin epegRNA expression plasmids for each target site were transfected into HEK293 cells. Droplet digital PCR (ddPCR) is a form of quantitative PCR that reports highly accurate copy number of genomic inserts over a wide range of template concentrations.

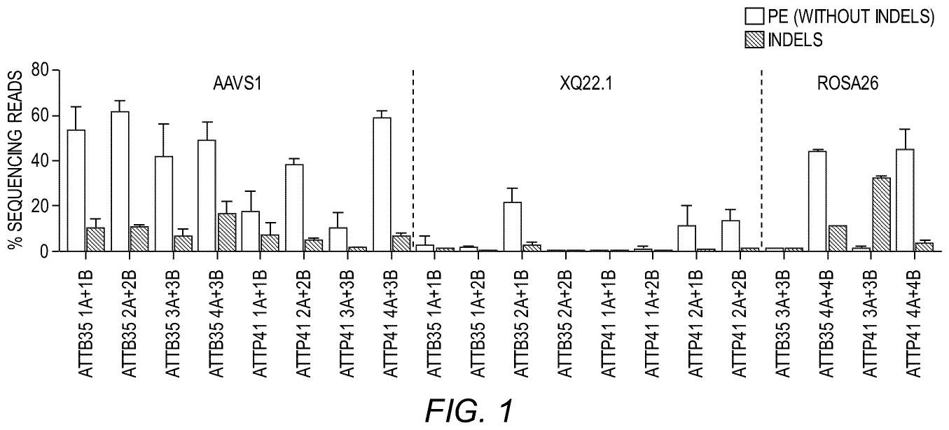

Prime editing insertion of att sites ranged from 0.3% to 62.1% as measured by amplicon sequencing using genomic primers flanking the insert (FIG. 1) and confirmed by ddPCR. Clonal HEK293 cell lines were generated, which included the most efficient and accurate epegRNA pairs targeted to AAVS1 and ROSA26, which were used to compare integration efficiency of PhiC31 integrase variants. Wild-type PhiC31 integrase was compared to three hyperactive variants called P1, P2, and P3 (Keravala et al. (2009) Mol. Ther. 17 (1):112-20). Two of these three variants (P1 and P3) demonstrated increased activity measured by ddPCR relative to the wild-type integrase at both loci with integration efficiencies for P3 reaching 11.1% and 3.0% at ROSA26 and AAVS1, respectively (FIG. 1). In place of using a cell line containing a pre-inserted att site, PE and integrase expression plasmids were combined in a single transfection. This strategy allowed for the insertion of an att site using PE followed by the insertion of the donor plasmid by PhiC31 integrase at the target sequence. PE5max and epegRNA plasmids were co-transfected to insert the 41 bp PhiC31 attP site at ROSA26 along with a 6.6 kb donor containing the attB site and a helper expressing PhiC31 integrase. Notably, PhiC31 insertion of the donor plasmid was detected for all four integrase variants with a favorable efficiency of 1.9% for P3 compared to 0.54% for wild-type. This demonstrated that hyperactive variants outperform wild-type PhiC31 integrase during PE-mediated gene insertion. Based upon these results, it was reasoned that directed evolution of PhiC31 variants may further increase the efficiency of gene insertion.

The prior directed evolution campaigns that generated P1, P2, and P3 required laborious steps of mutagenesis, gene expression, screening, and replication. Alternatively, PACE rapidly and iteratively selects, replicates, and mutates genes at rates of >70 generations per day without human intervention (Esvelt et al. (2011) Nature 472:499-503). PACE achieves selection by linking the desired activity of the gene of interest to the production of infectious phage progeny.

Expression of M13 phage's gene III (gIII) is proportionally related to proliferation. Using an accessory plasmid (AP), gIII expression is solely driven by the desired activity of the evolving gene. Each selection phage (SP) encodes the gene to be evolved. Phage encoding active variants of the gene produce infectious progeny, and phage encoding less-active variants fail to produce as many progeny and become outcompeted. Beneficial mutations multiply with successful phage life cycles. An inducible mutagenesis plasmid (MP) increases the mutation rate by decreasing proofreading and increasing error-prone bypass during replication. During PACE, host cells containing the AP, CP, and MP are continually added to a fixed-volume flask (called the ‘lagoon’) where they encounter phage. After infection, host cells begin expressing the integrase protein from the SP. A successfully evolved integrase, bearing beneficial mutations enhancing its activity on the attP site, aligns the gIII promoter on the AP. Activated gIII expression leads to propagation of the SP that encoded the improved integrase. These SP are again mutated during the next cycle of DNA replication, effectively generating a new library from the fittest variants of the previous library. Fresh host cells are then infected by this new library within the lagoon and the process is repeated. The proliferation of the phage scales with increasing levels of gIII expression over a range of two orders of magnitude. This establishes a competition between mutation library members resulting in the most active variants outcompeting the others.

To adapt PACE to improve integrase activity, the integrase was encoded on a selection phage (SP) and pIII expression was linked to a successful crossover event between the attB and attP sites. A promoter and the amino-terminus of gene III (gIII encodes pIII), called the leader sequence, followed by the attP site were placed on a on a complementary plasmid (CP). An accessory plasmid (AP) contained the attB site and the remaining portion of gIII (N1-C domains). During a crossover event between the attB and attP, the promoter and leader sequence become aligned with gIII that subsequently leads to expression. The resulting attL site is encoded within a flexible loop so as not to disrupt the function of pIII. Successful events require precise and directional recombination of the attL site.

PACE experiments were initiated with SP encoding PhiC31, P1, P2, and P3 and run for 212 hours (222 cycles of evolution) as stringency was increased incrementally by decreasing gIII promoter strength and increasing the flow rate. Different host cells containing CP with four gIII promoters were sequentially added to the lagoon. In order from strongest to weakest, promoters ProB, ProA, Pro3, and Prol were used to challenge the evolving integrases. Because each recombination event produces low levels of pIII when using a weak gIII promoter, only highly active integrase variants capable of recombining more plasmids than less active variants can generate enough pIII to make sufficient infectious progeny to maintain in the system. Less active integrase variants fail to perform enough recombination events to produce sufficient numbers of progeny and are drained from the system. To further challenge the evolving integrases, the flow rate was increased from 2 to 3 lagoon volumes per hour, thereby reducing the time the integrase variants had to generate progeny. The presence of the phage in each lagoon was monitored by collecting the media outflow and performing the activity-independent plaque assay. Dilutions of media containing phage were plated on the S2208 E. coli strain that produces blue plaques that were counted to calculate phage concentration. For each of the four host cell transfers, outflow media was collected at 0, 1, 2, and 4 hour time points and every 4 hours afterwards. The expected pattern of an initial drop in titer was observed as many of the early phage were drained. This was followed by a gradual increase in titer, presumably due to the integrase variants accumulating beneficial mutations. Next, phage numbers plateaued, likely due to the integrase variants reaching a point of hyperactivity that they were no longer challenged by the current level of stringency, necessitating increases in stringency for continued productive evolution. Isolated phage were selected at time points throughout the IntePACE experiment and recombination activity was separately measured using the overnight phage enrichment assay. Cells containing the AP and CP using the ProA promoter to drive gIII were inoculated with 10⁵ PFU/ml of purified phage isolated from IntePACE. The cells were cultured in the presence of the phage overnight and the next day the phage concentration was measured using the activity-independent plaque assay, similar to above. Time points containing phage encoding hyperactive integrase variants were expected to recombine more AP and CP and generate more phage progeny than phage encoding unevolved integrase. This method allows for individual time points to be directly compared. Evolved libraries increased efficiency by 15.4-, 70.2-, and 16.4-fold over P1, P2, and P3, respectively, as measured by overnight phage enrichment assay. To test activity in human cells, evolved integrase variants were isolated from single plaques and cloned into expression plasmids. A plasmid-based ‘flipper’ assay was designed in which a crossover event between the attP and attB sites flips the orientation of ZsGreen to align with the Hi promoter and activate expression. In agreement with results from E. coli, increased numbers of ZsGreen expressing cells were observed for evolved integrases, with the greatest improvement by P2 from 9.9% to 44.9%.

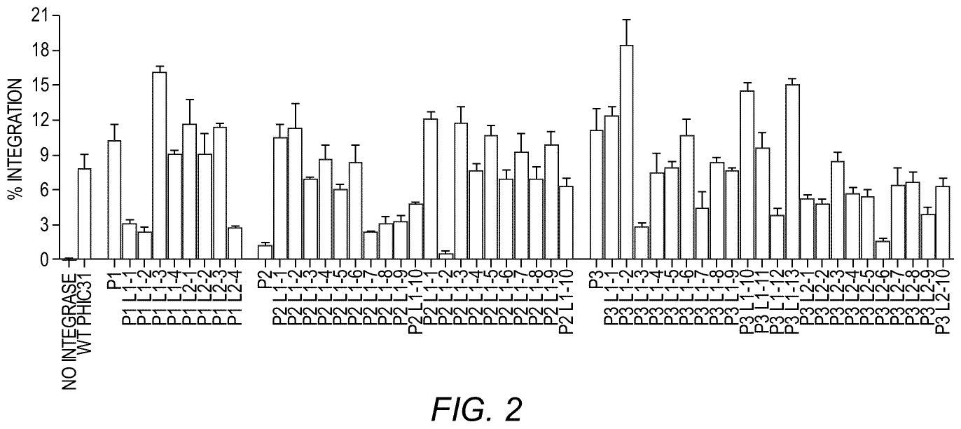

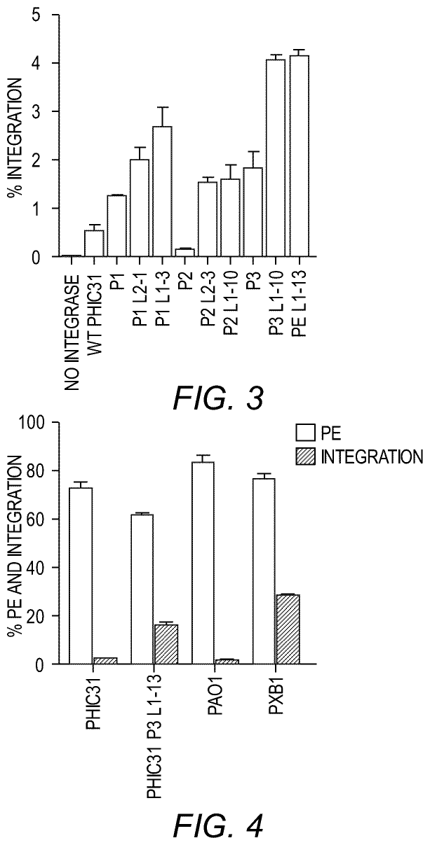

Evolved variants (Table 12) were compared in clonal HEK293 cell lines with a pre-inserted PhiC31 attP site at the AAVS1 or ROSA26 locus. Notably, gene insertion by evolved integrases outcompeted their starting variants. The greatest improvement was between P2 (1.3%) and a 36-hour evolved variant P2-L2-3 (11.8%). The highest integration rates came from a P3 variant (L1-2) that integrated 18.4% at the ROSA26 site (FIG. 2). Similarly, evolved integrases co-transfected with prime editing components for attP insertion resulted in higher gene targeting efficiencies. Specifically, a 36-hour variant evolved from P2 (L1-10) increased by 10.5-fold and a 184-hour variant from P3 (L1-13) reached 4.2% efficiency at the ROSA26 target sequence (up from 1.9% from the P3 starting variant) (FIG. 3).

TABLE 12

| Mutant | N-terminal Addition¹ | Internal Mutations² |
|---|---|---|
| Wild-Type | — | — |
| PhiC31 | | |
| P1 | MIQGVVSG (SEQ ID | M1E, D2V, V41I |
| | NO: 3) | |
| P2 | | D32A, D36A, D44A |
| P3 | MTMITPSAQLTLTK | M1V, D2V, D32A, D36A, V41I, D44A |
| | GNKSWSSLVTAAS | |
| | VLEFATMIQGVAG | |
| | (SEQ ID NO: 5) | |
| P1 L1-1 | MIQEVVSG (SEQ ID | M1V, D2V, V41I, R49R, M278L, L319L, |
| | NO: 4) | F527F |
| P1 L1-2 | MIQGVVSG (SEQ ID | M1V, D2V, V41I, L229L, G344V |
| | NO: 3) | |
| P1 L1-3 | MIQGVVSG (SEQ ID | M1V, D2V, V41I, E431V |
| | NO: 3) | |
| P1 L2-1 | MIQGVVSG (SEQ ID | M1V, D2V, V41I, R43R, P230S, Q355Q, |
| | NO: 3) | K520K |
| P1 L2-2 | MIQGVVSG (SEQ ID | M1V, D2V, V41I, V117V, H228Y, A255G, |
| | NO: 3) | A580V, D594D |
| P1 L2-3 | MIQGVVSG (SEQ ID | M1V, D2V, V41I, G505V, P587P, D594N |
| | NO: 3) | |
| P2 L1-1 | | D32A, A199T, P535P |
| P2 L1-2 | | D252G |
| P2 L1-3 | | D32A, D36A, D44A, S396N, R551R |
| P2 L1-4 | | D32A, D36A, D44A, N406S, E512K |
| P2 L1-5 | | D32A, D36A, D44A, K197K, D254D, |
| | | G344D, A397T, R457R, A616V |
| P2 L1-6 | | D32A, D36A, D44A, S55S, E77E, I153I, |
| | | G344D, S359S, V552V, A585A |
| P2 L1-7 | | D32A, D36A, D44A, K266K, D362G |
| P2 L1-8 | | D32A, D36A, D44A, F231L, G505S, D592G |
| P2 L1-9 | | D32A, D36A, D44A, I103I, T302A, D362G, |
| | | G399G, E449D, E472D |
| P2 L1-10 | | D32A, D36A, D44A, E77E, I153I, H200H, |
| | | P274S, G344D, K382K, T536A |
| P2 L2-1 | | A498A |
| P2 L2-2 | | D32A, D36A, D44A, R320R, A333T, G429S, |
| | | A516T, A604S |
| P2 L2-3 | | D32A, D36A, D44A, A238A, R457R, P535L |
| P2 L2-4 | | E14G, D32A, D36A, D44A, E176D, A238A, |
| | | S396R, R478R, F553F, K586Q |
| P2 L2-5 | | D32A, D36A, D44A, E176D, L229L, A238A, |
| | | A333D, S359S |
| P2 L2-6 | | D32A, D36A, D44A, I74I, R96R, E176D, |
| | | A238A, A333D, E378K, D590G |
| P3 L1-1 | MTMITPSAQLTLTK | M1E, D2V, D32A, D36A, V41I, D44A |
| | GNKSWSSLVTAAS | |
| | VLEFATMIQGVAG | |
| | (SEQ ID NO: 5) | |
| P3 L1-2 | MTMITPSAQLTLTK | M1E, D2V, D32A, D36A, V41I, D44A, |
| | GNKSWSSLVTAAS | S235S, L364M |
| | VLEFATMIQGVAG | |
| | (SEQ ID NO: 5) | |
| P3 L1-3 | MTMITPSAQLTLTK | M1E, D2V, D32A, D36A, V41I, D44A, |
| | GNKSWSSLVTAAS | G346S, G505G |
| | VLEFATMIQGVAG | |
| | (SEQ ID NO: 5) | |
| P3 L1-4 | MTMIYPFPAQLTLT | MIE, D2V, D32A, D36A, V41I, D44A, |
| | KGNKSWSSLVTAA | R345R, D362N, L468L, V603I |
| | SVLEFATMIQGVAG | |
| | (SEQ ID NO: 6) | |
| P3 L1-5 | MTMITPSAQLTLTK | M1E, D2V, D32A, D36A, V41I, D44A, |
| | GNKSWSSLVTAAS | D362N, V612V |
| | VLEFATMIQGVAG | |
| | (SEQ ID NO: 5) | |
| P3 L1-6 | MTMITPSAQLTLTK | M1E, D2V, A24A, D36A, V41I, D44A, |
| | GNKSWSSLVTAAS | N196N, W448R, P609S |
| | VLEFATMIQGVAG | |
| | (SEQ ID NO: 5) | |
| P3 L1-7 | MTMITPSAQLTLTK | M1E, D2V, D32A, D36A, V41I, D44A, |
| | SNKSWNSLVTAAS | D362G, V563V, T600T |
| | VLEFATMIQGVAG | |
| | (SEQ ID NO: 7) | |
| P3 L1-8 | MTMITPSAQLTLTK | M1E, D2V, D32A, D36A, V41I, D44A, |
| | GNKSWSSLVTAAS | F231L, A410T, V603V |
| | VLEFATMIQGVAG | |
| | (SEQ ID NO: 8) | |
| P3 L1-9 | MTMITPSAQLTLTK | M1E, D2V, D32A, D36A, V41I, D44A, |
| | GNKSWSSLVTAAS | T262T, D362N, V393V, L436L |
| | VLEFATMIQGVAG | |
| | (SEQ ID NO: 5) | |
| P3 L1-10 | MTMITPSAQLTLTK | M1E, D2M, D32A, V41I, D44A, G45G, |
| | SNKSWSSLVTAAS | G264G, E331E, L351V, G445G, V563V, |
| | VLEFATMIQGVTG | A580V, V603V |
| | (SEQ ID NO: 9) | |
| P3 L1-11 | MTMITPSAQLTLTK | M1E, D2V, A24A, D32A, D36A, V41I, |
| | GNKSWSSLVTAAS | D44A, W438R, P609S |
| | VLEFATMIQGVAG | |
| | (SEQ ID NO: 5) | |
| P3 L1-12 | MTMITPSAQLTLTK | M1E, D2V, A24A, D32A, D36A, V41I, |
| | GNKSWSSLVTAAS | D44A, H240R, A333S, A340V, W448R, |
| | VLEFATMIQGVAG | T600S, P609S |
| | (SEQ ID NO: 5) | |
| P3-L2-1 | MTMITPSAQLTLTK | M1E, D2V, D32A, D36A, V41I, D44A, |
| | GNKSWSSLVTAAS | R347K, I424I |
| | VLEFATMIQGVAG | |
| | (SEQ ID NO: 5) | |
| P3-L2-2 | MTMITPSAQLTLTK | M1E, D2V, D32A, D36A, V41I, D44A, |
| | DNKSWSSLVTAAS | V322V, L351Q, D549D |
| | VLEFATMIQGVAG | |
| | (SEQ ID NO: 10) | |
| P3-L2-3 | MTMITPSAQLTLTK | M1E, D2V, D32A, D36A, V41I, D44A, |
| | GNKSWSSLVTAAS | S107S, D362N |
| | VLEFATMIQGVAG | |
| | (SEQ ID NO: 5) | |
| P3-L2-4 | MTMITPSAQLTLTK | MIE, D2V, S12N, D32A, D36A, V41I, D44A, |
| | SNKSWSSLVTAAS | P58P, I103I, E153V, E514E, A580V, V603A |
| | VLEFATMIQGVAG | |
| | (SEQ ID NO: 11) | |
| P3-L2-5 | MTMITPSAQLTLTK | M1E, D2V, D32A, D36A, V41I, D44A, |
| | GNKSWSSLVTAAS | R353R, D362N, L499L, T568T |
| | VLEFATMIQGVAG | |
| | (SEQ ID NO: 5) | |
| P3-L2-6 | MTMITPSAQLTLTK | M1E, D2V, S18N, D32A, D36A, V41I, D44A, |
| | SNKSWSSLVTAAS | V51M, I103I, I153V, A450D, V603A |
| | VLEFATMIQGVAG | |
| | (SEQ ID NO: 11) | |
| P3-L2-7 | MTMITPSAQLTLTK | M1E, D2V, D32A, D36A, V41I, D44A, I103I, |
| | SNKSWSSLVTAAS | I153V, S269N, A450D, E517E, V603A |
| | VLEFATMIQGVAG | |
| | (SEQ ID NO: 11) | |
| P3-L2-8 | MTMITPSAQLTLTK | M1E, D2V, D32A, D36A, V41I, D44A, I103I, |
| | SNKSWSSLVTAAS | I153V, G260G, L436L, A450D, L501L, |
| | VLEFATMIQGVAG | V603A |
| | (SEQ ID NO: 11) | |
| P3-L2-9 | MTMITPSAQLTLTK | M1E, D2V, D32A, D36A, V41I, D44A, I103I, |
| | SNKSWSSLVTAAS | I153V, A450D,E452K, V603A |
| | VLEFATMIQGVAG | |
| | (SEQ ID NO: 12) | |

¹Keravala et al. (2009) Mol. Ther. 17(1):112-120.
²Amino acid positions are with reference to the wild-type 605 amino acid PhiC31 protein (SEQ ID NO: 2).

Using the optimized prime editing parameters for inserting att sites, evolved PhiC31 integration rates were compared with previously described integrases Bxb1 (Anzalone et al. (2022) Nat. Biotechnol. 40:731-740) and Pa01 (Durrant et al. (2023) Nat. Biotechnol. 41:488-499). Surprisingly, wild-type Bxb1 had greater efficiencies compared to evolved PhiC31 mutants (FIG. 4). It was reasoned that Bxb1 might serve as a superior starting point for evolution. Encouraged by the results with PhiC31, the same PACE strategy was adapted to increase Bxb1 integrase activity on its native att sites. The full Bxb1 attP site and minimal attB site (Ghosh et al. (2003) Mol. Cell 12(5):1101-11; Anzalone et al. (2022) Nat. Biotechnol. 40:731-740) were substituted for the PhiC31 att sites in the AP and CP, respectively (Tables 7 and 8). In total, phage were evolved for 9 days (approximately 276 cycles of mutagenesis) (Esvelt et al. (2011) Nature 472:499-503).

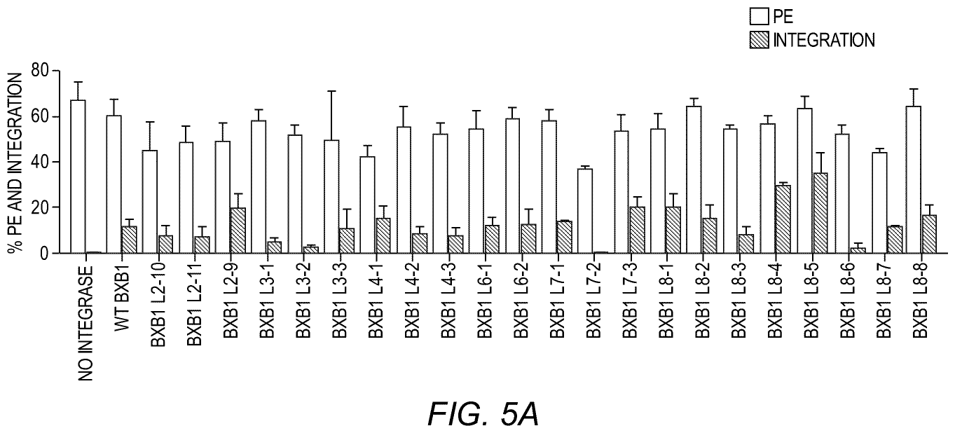

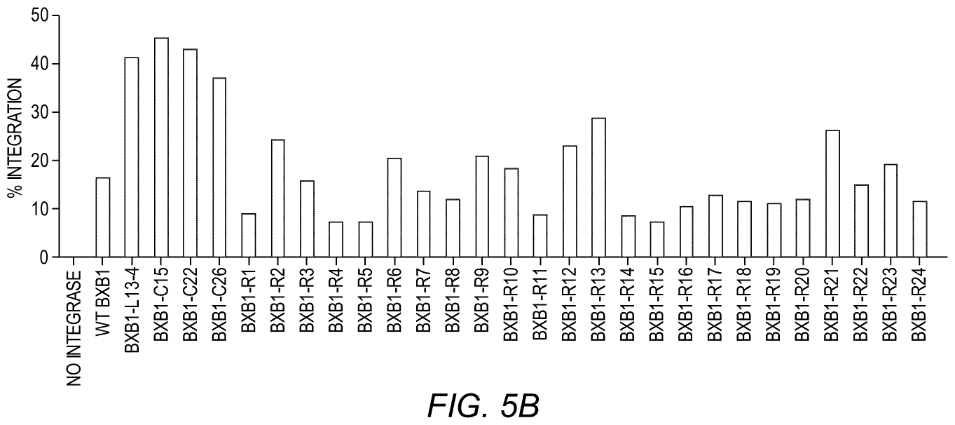

Similar to PACE with PhiC31, enrichment assays demonstrated that evolved variants of Bxb1 vastly improved in the bacterial system. New variants (Table 13) were cloned into mammalian expression plasmids which were then co-transfected with a donor into a clonal HEK293 cell line containing a pre-inserted Bxb1 attB site at ROSA26. It was observed that efficiencies up to 21% with the highest activity variant (Bxb1-L8-5) were 2.5-fold over wild-type Bxb1 integrase. Simultaneous prime editing and evolved Bxb1 integrase-mediated gene insertion resulted in efficiencies ranging from 2.1%-35.4%, up to a 3.1-fold increase in efficiency over wild-type Bxb1 (FIG. 5A and FIG. 5B). Additional rounds of evolution did not result in variants with increased efficiency.

TABLE 13

| Bxb1 Mutant | Mutations |
|---|---|
| Wild-Type Bxb1 | — |
| Bxb1-L4-1 | H111P, E434G |
| Bxb1-L6-1 | T285A |
| Bxb1-L6-2 | G156G, A369P |
| Bxb1-L4-2 | R63K, R88R, A110A, P295P |
| Bxb1-L4-3 | D36A, R63K, P292P, L479I |
| Bxb1-L2-9 | H95Y |
| Bxb1-L2-10 | V5V, S106S, E434G |
| Bxb1-L2-11 | D51D, H189N |
| Bxb1-L3-1 | L164L, H203Y, Q480STOP |
| Bxb1-L3-2 | F67S, V187I, A261T |
| Bxb1-L3-3 | L4I |
| Bxb1-L7-3 | I87L, V187V, A218A, E419E, A425T, E483E |
| Bxb1-L8-1 | R63K, A119S, P295P, E361E, M499T |
| Bxb1-L8-2 | R63K, V122M, P295P |
| Bxb1-L8-3 | R63K, A347E |
| Bxb1-L8-4 | V40I, R63K, V122M, P295P, R319K, S428S |
| Bxb1-L8-5 | R63K, V122M, P295P, R487R |
| Bxb1-L8-7 | L278L, P295P, P332H, A369T, V380I |
| Bxb1-L8-8 | E42K, R63K, D99N, E133E, A145T, K153K, P295P, A369E, |
| | R494S |
| Bxb1-L9-1 | V40A, R63K, H89G, Q191Q, P295P, L302L, C307C |
| Bxb1-L9-2 | V40V, R63K, P295P, R362K, G370G, G468G |
| Bxb1-L9-3 | G209G, A288V, A311V, A398S, R416K, T453I, H496N, E42K, |
| | G209G |
| Bxb1-L9-4 | A288V, A311V, A398S, R416K, T453I, H496N |
| Bxb1-L9-5 | S18S, R63K, E69E, I87V, P295P, P332H, H334R, A369E |
| Bxb1-L9-6 | S18S, E42K, R63K, E69E, E133E, P295P, P332H, H334R |
| Bxb1-L9-7 | P268P, A288V, A311V, D359D, T388M, T453I, V466V, H496N |
| Bxb1-L9-8 | A288V, A311V, R319G, A398S, R416K, T453I, H496N, E434G |
| Bxb1-L9-9 | E42K, A288V, A311V, A398S, R416K, T453I, H496N, E434G |
| Bxb1-L10-1 | H95Y, R287R, K313R |
| Bxb1-L10-2 | H95Y, A280T |
| Bxb1-L10-3 | H95Y, V264A |
| Bxb1-L10-4 | H95Y, T254S |
| Bxb1-L10-5 | V46V, H95Y, V179A, R223R |
| Bxb1-L10-6 | H95Y, V264A, E434G |
| Bxb1-L10-7 | H95Y, A62A, V466M |
| Bxb1-L11-1 | A280A, A360T |
| Bxb1-L11-2 | L61F, E434G |
| Bxb1-L11-3 | L174L, S231S, A288T, E434G, T463I |
| Bxb1-L11-4 | V40V, A130A, V283V, A288T, E434G, G468D |
| Bxb1-L11-5 | E229K, K313K, A405A, T453I |
| Bxb1-L11-6 | L90L, E229K, N251K, D359N, A360T, V375V, R494R |
| Bxb1-L11-7 | Q92Q, V179I, R181K, E229K, M239I, A360T, R444L |
| Bxb1-L12-1 | E69A, I87L, N251N, H321P, D355N |
| Bxb1-L12-2 | D51N, I87L, R272Q, G489G |
| Bxb1-L12-3 | I87L, del L488 frameshift |
| Bxb1-L12-4 | I87L, V105A, H321N, S328S, R397R, A411V |
| Bxb1-L12-5 | R79R, I87L, Q484K |
| Bxb1-L12-6 | I87L, I137I, F331S, R409H, del L488 frameshift |
| Bxb1-L13-1 | E69A, H95Y, R461R |
| Bxb1-L13-2 | H95Y |
| Bxb1-L13-3 | H95Y, L282L |
| Bxb1-L14-1 | I87L, A369P, F476F |
| Bxb1-L14-2 | G34D, I87L, T166I, E229K, A369P, T435T, G489G |
| Bxb1-L14-3 | I87L, R88R, R140R, K333K, A369P, A414A, V466M, F476F |
| Bxb1-L15-1 | I87L, A369S, E434G |
| Bxb1-L15-2 | I87L, A369P, A414V, A425A, E434G |
| Bxb1-L15-3 | R85R, I87L, A248T, V306V, A369P, A415S, E434G |
| Bxb1-L16-1 | I87L, P160P, V175V, V187V, A218A, E281E, V353I, E419E, |
| | A425T, E483E |
| Bxb1-L16-2 | I87L, Q92H, P160P, V175V, P178P, V187V, A218A, E281E, |
| | V353I, E419E, A425T, E483E |
| Bxb1-L16-3 | E24E, I87L, H100N, V187V, A218A, E419E, A425T, E483E |
| Bxb1-L16-4 | I87L, V187V, A218A, A369E, E419E, A425T, E483E |
| Bxb1-L16-5 | I87L, V187V, A218A, E419E, A425T, E434G, E483E |
| Bxb1-c1 | — |
| Bxb1-c2 | I87L |
| Bxb1-c3 | H95Y |
| Bxb1-c4 | V122M |
| Bxb1-c5 | A369P |
| Bxb1-c6 | E434G |
| Bxb1-c7 | I87L, H95Y |
| Bxb1-c8 | I87L, V122M |
| Bxb1-c9 | I87L, A369P |
| Bxb1-c10 | I87L, E434G |
| Bxb1-c11 | H95Y, V122M |
| Bxb1-c12 | H95Y, A369P |
| Bxb1-c13 | H95Y, E434G |
| Bxb1-c14 | V122M, A369P |
| Bxb1-c15 | V122M, E434G |
| Bxb1-c16 | A369P, E434G |
| Bxb1-c17 | I87L, H95Y, V122M |
| Bxb1-c18 | I87L, H95Y, A369P |
| Bxb1-c19 | I87L, H95Y, E434G |
| Bxb1-c20 | I87L, V122M, A369P |
| Bxb1-c21 | I87L, V122M, E434G |
| Bxb1-c22 | I87L, A369P, E434G |
| Bxb1-c23 | H95Y, V122M, A369P |
| Bxb1-c24 | H95Y, V122M, E434G |
| Bxb1-c25 | H95Y, A369P, E434G |
| Bxb1-c26 | V122M, A369P, E434G |
| Bxb1-c27 | I87L, H95Y, V122M, A369P |
| Bxb1-c28 | I87L, H95Y, V122M, E434G |
| Bxb1-c29 | I87L, H95Y, A369P, E434G |
| Bxb1-c30 | I87L, V122M, A369P, E434G |
| Bxb1-c31 | H95Y, V122M, A369P, E434G |
| Bxb1-c32 | I87L, H95Y, V122M, A369P, E434G |
| Bxb1-c33 | L4I, R63K, I87L, H95Y, H111P, V122M, A280T, A369P, E434G, |
| | V466M |
| Bxb1-c34 | R63K, V122M |
| Bxb1-R1 | H189Y |
| Bxb1-R2 | D45G |
| Bxb1-R3 | V466L |
| Bxb1-R4 | V76I |
| Bxb1-R5 | R63K, I87V, H100H, N194D, P295P, C307C |
| Bxb1-R6 | R57K, R63K, P295P |
| Bxb1-R7 | V168I |
| Bxb1-R8 | H89Y, A360V |
| Bxb1-R9 | D36N |
| Bxb1-R10 | E445Q |
| Bxb1-R11 | D51G |
| Bxb1-R12 | R181R, A341D |
| Bxb1-R13 | R287H, A425A |
| Bxb1-R14 | V50I, V306A |
| Bxb1-R15 | V366I, R426K |
| Bxb1-R16 | A261S |
| Bxb1-R17 | V74I |
| Bxb1-R18 | D359A, L387L |
| Bxb1-R19 | L263I |
| Bxb1-R20 | Q70H |
| Bxb1-R21 | A49S |
| Bxb1-R22 | D51E |
| Bxb1-R23 | I75V |
| Bxb1-R24 | V175A |

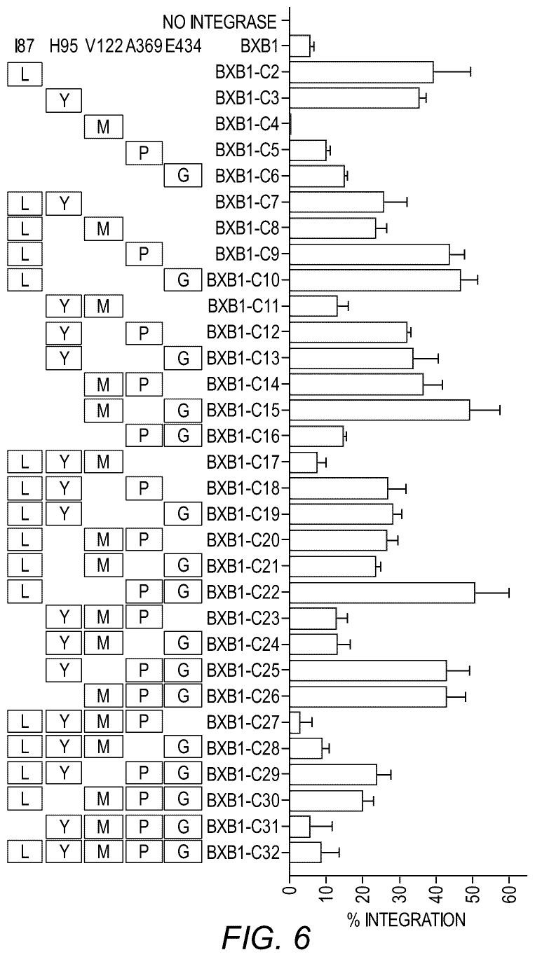

Five mutations from top-performing Bxb1 integrase variants isolated from PACE were assembled in all combinations to generate new mutants. To compare activity between mutants independent of PE, helper plasmids encoding combination mutants were co-transfected with a donor plasmid into a clonal HEK293 cell line with a pre-inserted attB site at ROSA26. Notably, combination mutants increased activity over wild-type Bxb1 integrase by up to 9.2-fold (FIG. 6). A variety of synergistic and antagonistic effects were observed. Of the five mutations, I87L and H95Y had the greatest individual effect on integration efficiency (6.4 and 7.1-fold higher than wild-type Bxb1 integrase, respectively), but when combined they were less effective (4.7-fold higher than wild-type Bxb1 integrase). Mutations A369P and E434G had a modest effect on integration activity on their own but exhibited a synergistic effect with every other mutation tested, including V122M, which decreased activity relative to wild-type Bxb1 integrase on its own.

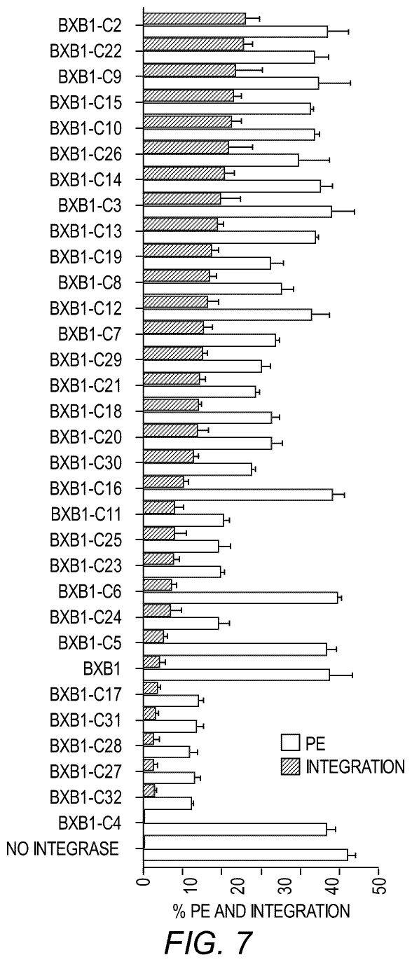

PE was subsequently used to insert the attB site at ROSA26 in HEK293 cells by co-transfecting PE plasmids with combination mutant helper plasmids and a donor plasmid. When combined with PE, combination mutants demonstrated increased activity over wild-type Bxb1 integrase up to 6-fold (FIG. 7). Different patterns of activity were observed for the combination mutants between experiments performed with and without PE (Table 14). For example, Bxb1-c25 had high activity on the clonal cell line and low activity when paired with PE, indicating that the mutations had a negative effect on PE which in turn decreased the integrase's insertion efficiency despite being hyperactive. Similarly, in all cases where H95Y and V122M were combined, PE efficiency decreased significantly (2.3- to 4-fold relative to wild-type Bxb1 integrase). It has been previously shown that the att site found on the epegRNA expression plasmid could recombine the att site on the donor plasmid, rendering both plasmids nonfunctional. This is expected to reduce the amount of epegRNA available for PE as well as reduce the number of donor plasmids available for integrase insertion. To reduce the effect of Bxb1 integrase recombining these plasmids, the epegRNA expression plasmids herein contained a partial att site. During twin PE, although each inserted 3′ single strand flap contains only part of the att site, there is enough overlapping sequence to form a double-stranded intermediate. The remaining single-stranded sequence of the att sites can subsequently be filled in to promote insertion of the full att site. While this strategy appears sufficient for preventing unwanted wild-type Bxb1 integrase recombination, it is speculated that certain hyperactive integrase mutants gained activity on the partial att site sequence and recombined the epegRNA expression plasmids, which may explain the reduction in PE efficiency.

AlphaFold modeling of Bxb1 integrase as a dimer revealed that three mutations I87L, H95Y and V122M are in close proximity to each other on the catalytic domain. Mutations A369P and E434G are located together proximal to a predicted leucine zipper motif suggesting they may affect dimerization.

Higher transfection rates were observed for HEK293T compared to HEK293 cells. Top variants were re-assayed using HEK293T cells resulting in insertion efficiencies up to 44.1%, with combination mutants outperforming parent variants (FIG. 7). Furthermore, No off-target insertions were detected by genomic PCR at predicted Bxb1 pseudo sites (Anzalone et al. (2022) Nat. Biotechnol. 40:731-740; Yarnall et al. (2023) Nat. Biotechnol. 41:500-512) Combination mutants were evolved using PACE but variants with further increases in efficiency were not recovered.

The programmable addition via site-specific targeting elements (PASTE) system has been described, which uses single PEG prime editing and a fusion of Bxb1 to the nCas9-RT (Yarnall et al. (2023) Nat. Biotechnol. 41:500-512). Similar to others, lower prime editing efficiencies were observed using a single pegRNA (Wang et al. (2022) Nat. Methods 19(3):331-340; Anzalone et al. (2022) Nat. Biotechnol. 40:731-740), with PASTE prime editing and integration into the ROSA26 locus reaching 11.2% and 2.4%, respectively. PASTE was improved by incorporating twin prime editing pegRNA and it was observed that the fused integrase architecture indeed outperformed traditional twin prime editing with wild-type Bxb1. However, neither traditional PASTE nor twin prime editing PASTE reached the levels of the evolved Bxb1 integrase. Several hyperactive mutations were introduced into both PASTE and twin prime editing PASTE but a further increase in efficiency was not observed.

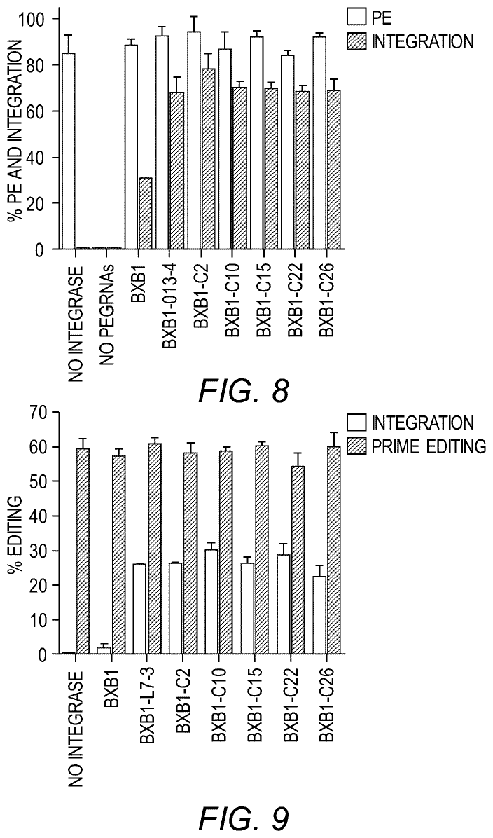

To exclude the influence of incomplete transfection efficiency, top evolved variants were assayed in HEK293T cells and sorted for DsRed expression. These optimized conditions resulted in insertion rates greater than 80% of target sites (FIG. 8). In a therapeutic context, given that normal cells contain a target site on each of two chromosomes, this efficiency equates to a >96% probability that a cell will receive at least one insertion.

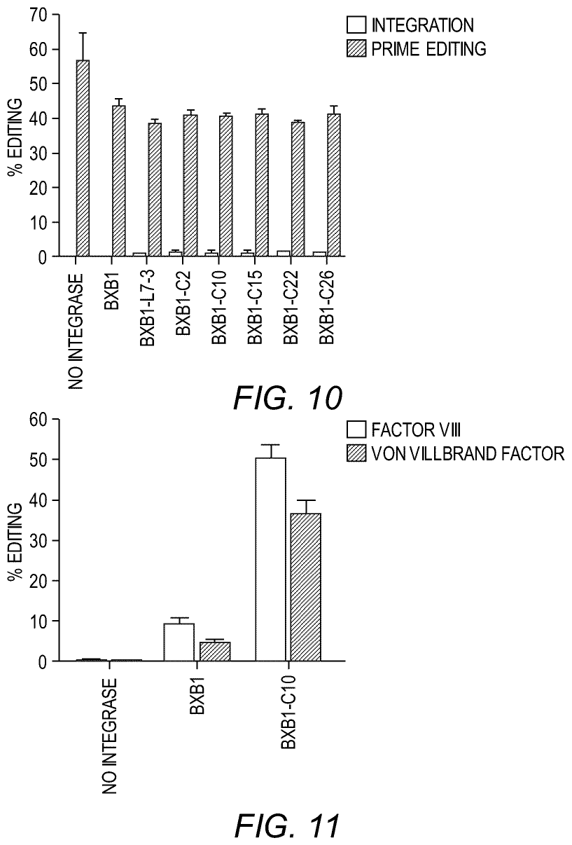

Evolved Bxb1 variants were active on HeLa and K562 cells. Surprisingly, although prime editing was efficient in HeLa cells (>40%), integration was not detected using wild-type Bxb1 integrase. However, evolved integrases successfully performed integration, albeit at low levels. In K562 cells, evolved integrases outperformed wild-type Bxb1 by 11.2-fold (2.7% vs 30.3%; FIG. 9 and FIG. 10).

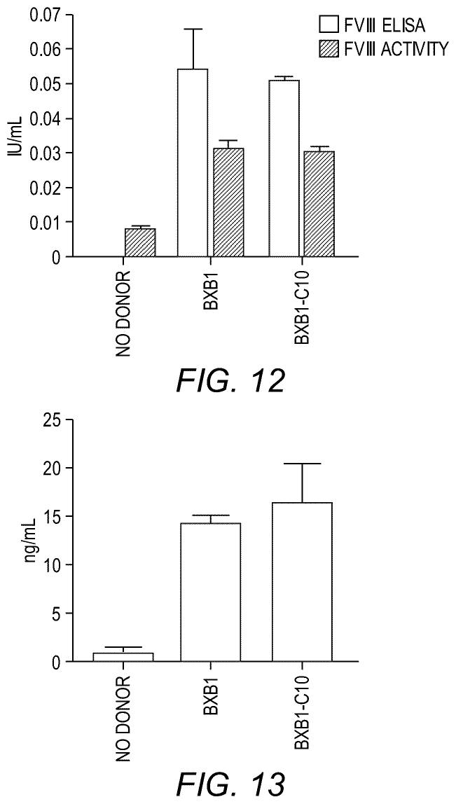

Integrase insertion of large cargos would enable therapies inaccessible to current methods. Specifically, the full-length Human Factor VIII (FVIII) gene (7 kb cDNA) and Von Willebrand Factor (VWF) gene (8.5 kb cDNA) are not amenable for HDR, AAV, or lentiviral delivery. Prime editing and integrase insertion of 14 kb FVIII (50.5%) and 15.7 kb VWF (36.4%) expression cargos were measured, and expression was verified by ELISA and Factor X activation (FIG. 11 to FIG. 13).

Active insertion by integrases does not rely on host repair and is highly specific for the att sequence that is not present in the unmodified human genome. Importantly, it has been shown that the efficiency of targeted insertion using evolved integrases and prime editing exceeds current approaches. A key finding was that evolved integrases outcompeted wild-type Bxb1 particularly well during pre-optimized transfection conditions (>6 fold) (Table 14) indicating that this technology is suitable for in-vivo gene delivery where tissue transfection efficiency can be challenging.

TABLE 14

| | |
|---|---|
| Prime Editing | Integration |

| | Editing | | Editing | |
|---|---|---|---|---|
| Integrase | Frequency | Stdev. | Frequency | Stdev. |
| No Integrase | 43.51% | 1.49% | 0.01% | 0.01% |
| Bxb1-c4 | 38.97% | 1.94% | 0.04% | 0.01% |
| Bxb1-c32 | 10.47% | 0.38% | 2.41% | 0.60% |
| Bxb1-c27 | 11.15% | 1.11% | 2.42% | 0.61% |
| Bxb1-c28 | 10.06% | 1.62% | 2.45% | 1.02% |
| Bxb1-c31 | 11.49% | 1.29% | 2.80% | 0.48% |
| Bxb1-c17 | 11.95% | 0.84% | 3.26% | 0.70% |
| WT Bxb1 | 39.57% | 4.85% | 3.66% | 1.19% |
| Bxb1-c5 | 39.05% | 1.91% | 4.45% | 0.68% |
| Bxb1-c24 | 16.13% | 2.13% | 6.08% | 2.04% |
| Bxb1-c6 | 41.33% | 0.63% | 6.17% | 0.97% |
| Bxb1-c23 | 16.57% | 0.75% | 6.57% | 1.21% |
| Bxb1-c25 | 15.97% | 2.50% | 6.88% | 2.43% |
| Bxb1-c11 | 17.20% | 0.94% | 6.92% | 1.77% |
| Bxb1-c16 | 40.27% | 2.49% | 8.74% | 0.90% |
| Bxb1-c30 | 22.93% | 0.78% | 10.89% | 0.98% |
| Bxb1-c20 | 27.17% | 2.41% | 11.68% | 2.15% |
| Bxb1-c18 | 27.25% | 1.74% | 11.94% | 0.49% |
| Bxb1-c21 | 23.90% | 0.98% | 12.22% | 1.04% |
| Bxb1-c29 | 25.26% | 1.68% | 12.73% | 0.88% |
| Bxb1-c7 | 28.25% | 0.75% | 13.05% | 1.87% |
| Bxb1-c12 | 35.80% | 3.68% | 13.83% | 2.20% |
| Bxb1-c8 | 29.40% | 2.63% | 14.39% | 1.20% |
| Bxb1-c19 | 27.07% | 2.75% | 14.76% | 1.26% |
| Bxb1-c13 | 36.53% | 0.68% | 16.07% | 1.12% |
| Bxb1-c3 | 39.99% | 4.86% | 16.66% | 4.04% |
| Bxb1-c14 | 37.80% | 2.45% | 17.59% | 1.93% |
| Bxb1-c26 | 33.05% | 6.52% | 18.35% | 4.97% |
| Bxb1-c10 | 36.48% | 0.89% | 19.09% | 1.78% |
| Bxb1-c15 | 35.55% | 0.60% | 19.19% | 1.66% |
| Bxb1-c9 | 37.34% | 6.71% | 19.91% | 5.51% |
| Bxb1-c22 | 36.49% | 2.85% | 21.44% | 1.89% |
| Bxb1-c2 | 39.05% | 4.50% | 21.85% | 2.94% |

TABLE 15

| | | | | | RT |
|---|---|---|---|---|---|
| | | Spacer | | | Template |
| | | SEQ ID | | | SEQ ID |
| Name | Spacer | NO: | Scaffold | RT Template | NO: |
| aav peg | aagtggttgataaacccacg | 248 | cr772 | ggggagcccaagggcacg | 289 |
| spacer1a | | | | ccctggcac | |
| attB35 | | | | | |
| aav peg | ccggccagtttctccacctg | 249 | cr772 | ggcgtgcccttgggctccc | 290 |
| spacer1b | | | | cgggcgcgt | |
| attB35 | | | | | |
| aav peg | caggtaaaactgacgcacgg | 250 | cr772 | ggggagcccaagggcacg | 291 |
| spacer2a | | | | ccctggcac | |
| attB35 | | | | | |
| aav peg | gcttccttacacttcccaag | 251 | cr772 | ggcgtgcccttgggctccc | 292 |
| spacer2b | | | | cgggcgcgt | |
| attB35 | | | | | |
| aav peg | gatcagtgaaacgcaccaga | 252 | cr772 | ggggagcccaagggcacg | 293 |
| spacer3a | | | | ccctggcac | |
| attB35 | | | | | |
| aav peg | gatggagccagagaggatcc | 253 | cr772 | ggcgtgcccttgggctccc | 294 |
| spacer3b | | | | cgggcgcgt | |
| attB35 | | | | | |
| aav peg | gcagctcaggttctgggaga | 254 | cr772 | ggggagcccaagggcacg | 295 |
| spacer4a | | | | ccctggcac | |
| attB35 | | | | | |
| aav peg | aagtggttgataaacccacg | 255 | cr772 | tggggtaacctttgagttctc | 296 |
| spacer1a | | | | tcagttgggggc | |
| attP41 | | | | | |
| aav peg | ccggccagtttctccacctg | 256 | cr772 | actgagagaactcaaaggtt | 297 |
| spacer1b | | | | accccagttggggc | |
| attP41 | | | | | |
| aav peg | caggtaaaactgacgcacgg | 257 | cr772 | tggggtaacctttgagttctc | 298 |
| spacer2a | | | | tcagttgggggc | |
| attP41 | | | | | |
| aav peg | gcttccttacacttcccaag | 258 | cr772 | actgagagaactcaaaggtt | 299 |
| spacer2b | | | | accccagttggggc | |
| attP41 | | | | | |
| aav peg | gatcagtgaaacgcaccaga | 259 | cr772 | tggggtaacctttgagttctc | 300 |
| spacer3a | | | | tcagttgggggc | |
| attP41 | | | | | |
| aav peg | gatggagccagagaggatcc | 260 | cr772 | actgagagaactcaaaggtt | 301 |
| spacer3b | | | | accccagttggggc | |
| attP41 | | | | | |
| xq22 peg | ctactctaattaaacctgta | 261 | cr772 | ggggagcccaagggcacg | 302 |
| spacer1a | | | | ccctggcac | |
| attB35 | | | | | |
| xq22 peg | taatgcctcatttaatctat | 262 | cr772 | ggcgtgcccttgggctccc | 303 |
| spacer1b | | | | cgggcgcgt | |
| attB35 | | | | | |
| xq22 peg | aatgagcaaactctagaatg | 263 | cr772 | ggggagcccaagggcacg | 304 |
| spacer2a | | | | ccctggcac | |
| attB35 | | | | | |
| xq22 peg | aatggtccccaggttacatt | 264 | cr772 | ggcgtgcccttgggctccc | 305 |
| spacer2b | | | | cgggcgcgt | |
| attB35 | | | | | |
| xq22 peg | ctactctaattaaacctgta | 265 | cr772 | tggggtaacctttgagttctc | 306 |
| spacer1a | | | | tcagttgggggc | |
| attP41 | | | | | |
| xq22 peg | taatgcctcatttaatctat | 266 | cr772 | actgagagaactcaaaggtt | 307 |
| spacer1b | | | | accccagttggggc | |
| attP41 | | | | | |
| xq22 peg | aatgagcaaactctagaatg | 267 | cr772 | tggggtaacctttgagttctc | 308 |
| spacer2a | | | | tcagttgggggc | |
| attP41 | | | | | |
| xq22 peg | aatggtccccaggttacatt | 268 | cr772 | actgagagaactcaaaggtt | 309 |
| spacer2b | | | | accccagttggggc | |
| attP41 | | | | | |
| rosa peg | togacaccaactctagtccg | 269 | cr772 | ggggagcccaagggcacg | 310 |
| spacer1a | | | | ccctggcac | |
| attB35 | | | | | |
| rosa peg | agtcgcttctogattatggg | 270 | cr772 | ggcgtgcccttgggctccc | 311 |
| spacer1b | | | | cgggcgcgt | |
| attB35 | | | | | |
| rosa peg | ccctgggogttgccctgcag | 271 | cr772 | ggcgtgcccttgggctccc | 312 |
| spacer2b | | | | cgggcgcgt | |
| attB35 | | | | | |
| rosa peg | tctcaaatggtataaaactc | 272 | cr772 | ggggagcccaagggcacg | 313 |
| spacer3a | | | | ccctggcac | |
| attB35 | | | | | |
| rosa peg | ggagcttagtcattcacctg | 273 | cr772 | ggcgtgcccttgggctccc | 314 |
| spacer3b | | | | cgggcgcgt | |
| attB35 | | | | | |
| rosa peg | atgcctggtagggatgcaaa | 274 | cr772 | ggggagcccaagggcacg | 315 |
| spacer4a | | | | ccctggcac | |
| attB35 | | | | | |
| rosa peg | caccaccaaagtgtagcatc | 275 | cr772 | ggcgtgcccttgggctccc | 316 |
| spacer4b | | | | cgggcgcgt | |
| attB35 | | | | | |
| rosa peg | togacaccaactctagtccg | 276 | cr772 | tggggtaacctttgagttctc | 317 |
| spacer1a | | | | tcagttgggggc | |
| attP41 | | | | | |
| rosa peg | agtcgcttctcgattatggg | 277 | cr772 | actgagagaactcaaaggtt | 318 |
| spacer1b | | | | accccagttggggc | |
| attP41 | | | | | |
| rosa peg | ccctgggogttgccctgcag | 278 | cr772 | actgagagaactcaaaggtt | 319 |
| spacer2b | | | | accccagttggggc | |
| attP41 | | | | | |
| rosa peg | tctcaaatggtataaaactc | 279 | cr772 | tggggtaacctttgagttctc | 320 |
| spacer3a | | | | tcagttgggggc | |
| attP41 | | | | | |
| rosa peg | ggagcttagtcattcacctg | 280 | cr772 | actgagagaactcaaaggtt | 321 |
| spacer3b | | | | accccagttggggc | |
| attP41 | | | | | |
| rosa peg | atgcctggtagggatgcaaa | 281 | cr772 | tggggtaacctttgagttctc | 322 |
| spacer4a | | | | tcagttgggggc | |
| attP41 | | | | | |
| rosa peg | caccaccaaagtgtagcatc | 282 | cr772 | actgagagaactcaaaggtt | 323 |
| spacer4b | | | | accccagttggggc | |
| attP41 | | | | | |
| rosa peg | atgcctggtagggatgcaaa | 283 | cr772 | taccgtacaccactgagac | 324 |
| spacer4a | | | | cgcggtggttgaccagaca | |
| Bxb1attP48 | | | | aacct | |
| rosa peg | caccaccaaagtgtagcatc | 284 | cr772 | gtctggtcaaccaccgcgg | 325 |
| spacer4b | | | | tctcagtggtgtacggtaca | |
| Bxb1attP48 | | | | aacct | |
| rosa peg | atgcctggtagggatgcaaa | 285 | cr772 | acgacggcggtctccgtcg | 326 |
| spacer4a | | | | tcaggatcat | |
| Bxb1attB38 | | | | | |
| rosa peg | caccaccaaagtgtagcatc | 286 | cr772 | acgacggagaccgccgtc | 327 |
| spacer4b | | | | gtcgacaagcc | |
| Bxb1attP38 | | | | | |
| rosa peg | atgcctggtagggatgcaaa | 287 | cr772 | CCTACATGCTCG | 328 |
| spacer 4a pa1 | | | | AAGGGCGTATGC | |
| attB | | | | GCC | |
| rosa peg | caccaccaaagtgtagcatc | 288 | cr772 | ACGCCCTTCGAG | 329 |
| spacer 4b pa1 | | | | CATGTAGGTCAC | |
| attB | | | | GG | |

TABLE 16

| | | PBS SEQ | | |
|---|---|---|---|---|
| Name | PBS | ID NO: | Linker | TevopreQ1 |
| aav peg spacer1a attB35 | gggtttatcaacc | 330 | GGAATGCC | tevopreQ1 |
| aav peg spacer1b attB35 | gtggagaaactgg | 331 | AATTAATG | tevopreQ1 |
| aav peg spacer2a attB35 | tgcgtcagtttta | 332 | AGGTTCAA | tevopreQ1 |
| aav peg spacer2b attB35 | gggaagtgtaagg | 333 | TCAAATGA | tevopreQ1 |
| aav peg spacer3a attB35 | ggtgcgtttcact | 334 | CTATAAGA | tevopreQ1 |
| aav peg spacer3b attB35 | tcctctctggctc | 335 | GTAATAAT | tevopreQ1 |
| aav peg spacer4a attB35 | cccagaacctgag | 336 | AATTAATT | tevopreQ1 |
| aav peg spacer1a attP41 | gggtttatcaacc | 337 | TTCAGACC | tevopreQ1 |
| aav peg spacer1b attP41 | gtggagaaactgg | 338 | AATTATAA | tevopreQ1 |
| aav peg spacer2a attP41 | tgcgtcagtttta | 339 | AAGGTCAC | tevopreQ1 |
| aav peg spacer2b attP41 | gggaagtgtaagg | 340 | CCTCAATT | tevopreQ1 |
| aav peg spacer3a attP41 | ggtgcgtttcact | 341 | TTATAATA | tevopreQ1 |
| aav peg spacer3b attP41 | tcctctctggctc | 342 | AACTGAAA | tevopreQ1 |
| xq22 peg spacer1a attB35 | aggtttaattaga | 343 | TGCCATAA | tevopreQ1 |
| xq22 peg spacer1b attB35 | gattaaatgaggc | 344 | CCGAAGAA | tevopreQ1 |
| xq22 peg spacer2a attB35 | tctagagtttgct | 345 | GCCCGGAT | tevopreQ1 |
| xq22 peg spacer2b attB35 | gtaacctggggac | 346 | GTCCGACA | tevopreQ1 |
| xq22 peg spacer1a attP41 | aggtttaattaga | 347 | TTCCCATC | tevopreQ1 |
| xq22 peg spacer1b attP41 | gattaaatgaggc | 348 | CGAAATGC | tevopreQ1 |
| xq22 peg spacer2a attP41 | tctagagtttgct | 349 | AAGGGACA | tevopreQ1 |
| xq22 peg spacer2b attP41 | gtaacctggggac | 350 | AAGAATTA | tevopreQ1 |
| rosa peg spacer1a attB35 | actagagttggtg | 351 | AATAGTAC | tevopreQ1 |
| rosa peg spacer1b attB35 | ataatcgagaagc | 352 | AAATAACG | tevopreQ1 |
| rosa peg spacer2b attB35 | cagggcaacgccc | 353 | GCTTAACT | tevopreQ1 |
| rosa peg spacer3a attB35 | ttttataccattt | 354 | TACACACG | tevopreQ1 |
| rosa peg spacer3b attB35 | gtgaatgactaag | 355 | AGAAAGAT | tevopreQ1 |
| rosa peg spacer4a attB35 | gcatccctaccag | 356 | ATCTCAGA | tevopreQ1 |
| rosa peg spacer4b attB35 | gctacactttggt | 357 | TATTATAA | tevopreQ1 |
| rosa peg spacer1a attP41 | actagagttggtg | 358 | AAACCCTT | tevopreQ1 |
| rosa peg spacer1b attP41 | ataatcgagaagc | 359 | AAATATAA | tevopreQ1 |
| rosa peg spacer2b attP41 | cagggcaacgccc | 360 | CTTTCAAA | tevopreQ1 |
| rosa peg spacer3a attP41 | ttttataccattt | 361 | TTCTACTA | tevopreQ1 |
| rosa peg spacer3b attP41 | gtgaatgactaag | 362 | AATAAGAA | tevopreQ1 |
| rosa peg spacer4a attP41 | gcatccctaccag | 363 | ATCTAATG | tevopreQ1 |
| rosa peg spacer4b attP41 | gctacactttggt | 364 | TAATAACG | tevopreQ1 |
| rosa peg spacer4a | gcatccctaccag | 365 | AAACCACA | tevopreQ1 |
| Bxb1attP48 | | | | |
| rosa peg spacer4b | gctacactttggt | 366 | CTCATCTT | tevopreQ1 |
| Bxb1attP48 | | | | |
| rosa peg spacer4a | gcatccctaccag | 367 | AACTTCGT | tevopreQ1 |
| Bxb1attB38 | | | | |
| rosa peg spacer4b | gctacactttggt | 368 | CCATATAA | tevopreQ1 |
| Bxb1attP38 | | | | |
| rosa peg spacer 4a pa1 attB | gcatccctaccag | 369 | ATCCTACA | tevopreQ1 |
| rosa peg spacer 4b pa1 attB | gctacactttggt | 370 | CCATATAA | tevopreQ1 |

### Example 3: Further Directed Evolution of Bxb1

The Bxb1 integrase of SEQ ID N: was further evolved as described herein to generate additional mutant Bxb1 integrases including combinations of amino acid substitutions (Table 17).

TABLE 17

| Bxb1 Mutant | Mutations |
|---|---|
| Bxb1-c35 | I87L, D45G |
| Bxb1-c36 | I87L, R57K, R63K |
| Bxb1-c37 | I87L, D36N |
| Bxb1-c38 | I87L, A341D |
| Bxb1-c39 | I87L, R287H |
| Bxb1-c40 | I87L, A49S |
| Bxb1-c41 | I87L, A341D, R287H |
| Bxb1-c42 | H95Y, D45G |
| Bxb1-c43 | H95Y, R57K, R63K |
| Bxb1-c44 | H95Y, D36N |
| Bxb1-c45 | H95Y, A341D |
| Bxb1-c46 | H95Y, R287H |
| Bxb1-c47 | H95Y, A49S |
| Bxb1-c48 | H95Y, A341D, R287H |
| Bxb1-c49 | A369P, E434G, D45G |
| Bxb1-c50 | A369P, E434G, R57K, R63K |
| Bxb1-c51 | A369P, E434G, D36N |
| Bxb1-c52 | A369P, E434G, A341D |
| Bxb1-c53 | A369P, E434G, R287H |
| Bxb1-c54 | A369P, E434G, A49S |
| Bxb1-c55 | A369P, E434G, A341D, R287H |
| Bxb1-c56 | I87L, A369P, E434G, D45G |
| Bxb1-c57 | I87L, A369P, E434G, R57K, R63K |
| Bxb1-c58 | I87L, A369P, E434G, D36N |
| Bxb1-c59 | I87L, A369P, E434G, A341D |
| Bxb1-c60 | I87L, A369P, E434G, R287H |
| Bxb1-c61 | I87L, A369P, E434G, A49S |
| Bxb1-c62 | I87L, A369P, E434G, A341D, R287H |
| Bxb1-c63 | V122M, A369P, E434G, D45G |
| Bxb1-c64 | V122M, A369P, E434G, R57K, R63K |
| Bxb1-c65 | V122M, A369P, E434G, D36N |
| Bxb1-c66 | V122M, A369P, E434G, A341D |
| Bxb1-c67 | V122M, A369P, E434G, R287H |
| Bxb1-c68 | V122M, A369P, E434G, A49S |
| Bxb1-c69 | V122M, A369P, E434G, A341D, R287H |
| Bxb1-c71 | V76I, E434G |
| Bxb1-c72 | V76I, A369P, E434G |
| Bxb1-c73 | I87L, E434G, D36E |
| Bxb1-c74 | V122M, E434G, D36E |
| Bxb1-c75 | V76I, I87L |
| Bxb1-c76 | V76I, I87L, A369P, E434G |
| Bxb1-c77 | V76I, V122M, A369P, E434G |
| Bxb1-c78 | I87L, V175A |
| Bxb1-c79 | I87L, V175A, A369P, E434G |
| Bxb1-c80 | V122M, V175A, A369P, E434G |
| Bxb1-c81 | R63K, I87L |
| Bxb1-c82 | R63K, I87L, A369P, E434G |
| Bxb1-c83 | R63K, V122M, A369P, E434G |
| Bxb1-c84 | I87L, N194D |
| Bxb1-c85 | I87L, N194D, A369P, E434G |
| Bxb1-c86 | V122M, N194D, A369P, E434G |
| Bxb1-c87 | I87L, V175A, N194D |
| Bxb1-c88 | I87L, V175A, N194D, A369P, E434G |
| Bxb1-c89 | V122M, V175A, N194D, A369P, E434G |
| Bxb1-c90 | I87L, V175A, D359A |
| Bxb1-c91 | I87L, V175A, D359A, A369P, E434G |
| Bxb1-c92 | V122M, V175A, D359A, A369P, E434G |
| Bxb1-c93 | R57K, I87L |
| Bxb1-c94 | R57K, I87L, A369P, E434G |
| Bxb1-c95 | R57K, V122M, A369P, E434G |
| Bxb1-c96 | D36E, V76I |
| Bxb1-c97 | V175A, N194D |
| Bxb1-c98 | R63K, V76I |
| Bxb1-c99 | D36N, R63K |
| Bxb1-c100 | V76I, N194D |
| Bxb1-c101 | D36N, V76I, I87L |
| Bxb1-c103 | V76I, I87L, R63K |
| Bxb1-c105 | I87L, A369P, E434G, D36E, V76I |
| Bxb1-c107 | D36N, R63K, V76I, I87L, A369P, E434G |
| Bxb1-c108 | D36N, R63K, V76I, V175A |
| Bxb1-c109 | V74I, I75V |
| Bxb1-c110 | V74I, V76I |
| Bxb1-c111 | V74I |
| Bxb1-c113 | I75V, V76I |
| Bxb1-c114 | V76I |
| Bxb1-c116 | I75V |
| Bxb1-c117 | I87L, D359A |
| Bxb1-c118 | I87L, D359A, A369P, E434G |
| Bxb1-c119 | V122M, D359A, A369P, E434G |
| Bxb1-c120 | L4I, I87L |
| Bxb1-c121 | I87L, H111P |
| Bxb1-c122 | I87L, A119S |
| Bxb1-c123 | I87L, R319K |
| Bxb1-c124 | I87L, A425T |
| Bxb1-c125 | I87L, V179A |
| Bxb1-c126 | V401, I87L |
| Bxb1-c127 | I87L, V466M |
| Bxb1-c128 | I87L, K313R |
| Bxb1-c129 | E42K, I87L |
| Bxb1-c130 | I87L, L479I |
| Bxb1-c131 | L4I, H95Y, A369P, E434G |
| Bxb1-c132 | H95Y, H111P, A369P, E434G |
| Bxb1-c133 | H95Y, A119S, A369P, E434G |
| Bxb1-c134 | H95Y, R319K, A369P, E434G |
| Bxb1-c135 | H95Y, A369P, A425T, E434G |
| Bxb1-c136 | H95Y, V179A, A369P, E434G |
| Bxb1-c137 | V40I, H95Y, A369P, E434G |
| Bxb1-c138 | H95Y, A369P, E434G, V466M |
| Bxb1-c139 | H95Y, K313R, A369P, E434G |
| Bxb1-c140 | E42K, H95Y, A369P, E434G |
| Bxb1-c141 | H95Y, A369P, E434G, L479I |
| Bxb1-c142 | L4I, I87L, A369P, E434G |
| Bxb1-c143 | I87L, H111P, A369P, E434G |
| Bxb1-c144 | I87L, A119S, A369P, E434G |
| Bxb1-c145 | I87L, R319K, A369P, E434G |
| Bxb1-c146 | I87L, A369P, A425T, E434G |
| Bxb1-c147 | L4I, V122M, A369P, E434G |
| Bxb1-c148 | H111P, V122M, A369P, E434G |
| Bxb1-c149 | A119S, V122M, A369P, E434G |
| Bxb1-c150 | V122M, R319K, A369P, E434G |
| Bxb1-c151 | V122M, A369P, A425T, E434G |
| Bxb1-c152 | L4I, V76I |
| Bxb1-c153 | V76I, H111P |
| Bxb1-c154 | V76I, A119S |
| Bxb1-c155 | L4I, D36N |
| Bxb1-c156 | D36N, H111P |
| Bxb1-c157 | D36N, A119S |
| Bxb1-c158 | L4I, A369P, E434G |
| Bxb1-c159 | H111P, A369P, E434G |
| Bxb1-c160 | A119S, A369P, E434G |
| Bxb1-c161 | V40I, A369P, E434G |
| Bxb1-c162 | E42K, A369P, E434G |

As demonstrated elsewhere herein, evolved Bxb1 integrases outcompeted wild-type Bxb1 (Tables 18 and 19).

TABLE 18

| | |
|---|---|
| Prime Editing | Integration |

| | Editing | | Editing | |
|---|---|---|---|---|
| Integrase | Frequency | Stdev. | Frequency | Stdev. |
| No Integrase | 49.11% | 1.55% | 0.00% | 0.00% |
| WT Bxb1 | 50.93% | 4.08% | 9.61% | 0.94% |
| Bxb1-c2 | 39.79% | 2.58% | 12.92% | 1.17% |
| Bxb1-c35 | 29.81% | 2.84% | 3.09% | 0.41% |
| Bxb1-c36 | 55.21% | 2.78% | 29.04% | 0.90% |
| Bxb1-c37 | 52.28% | 3.22% | 28.64% | 2.10% |
| Bxb1-c38 | 50.63% | 1.92% | 21.92% | 0.94% |
| Bxb1-c39 | 49.15% | 2.21% | 22.38% | 1.63% |
| Bxb1-c40 | 53.21% | 3.72% | 26.07% | 1.06% |
| Bxb1-c41 | 50.73% | 2.06% | 16.11% | 0.54% |
| Bxb1-c42 | 35.91% | 0.41% | 4.63% | 0.23% |
| Bxb1-c43 | 55.77% | 1.08% | 24.76% | 0.83% |
| Bxb1-c44 | 54.69% | 2.76% | 23.67% | 2.02% |
| Bxb1-c45 | 54.19% | 2.12% | 17.62% | 1.15% |
| Bxb1-c46 | 49.82% | 0.73% | 15.92% | 0.46% |
| Bxb1-c47 | 50.15% | 0.74% | 23.59% | 1.26% |
| Bxb1-c48 | 45.72% | 1.09% | 7.43% | 0.18% |
| Bxb1-c16 | 43.40% | 1.77% | 10.93% | 1.50% |
| Bxb1-c49 | 44.39% | 0.95% | 16.74% | 0.50% |
| Bxb1-c50 | 47.31% | 2.01% | 12.75% | 0.77% |
| Bxb1-c51 | 47.59% | 1.27% | 14.16% | 0.10% |
| Bxb1-c52 | 46.47% | 1.18% | 8.66% | 0.44% |
| Bxb1-c53 | 49.94% | 1.10% | 10.19% | 0.45% |
| Bxb1-c54 | 46.19% | 0.77% | 16.48% | 0.34% |
| Bxb1-c55 | 47.00% | 1.99% | 4.78% | 0.44% |
| Bxb1-c22 | 51.54% | 1.39% | 26.97% | 0.68% |
| Bxb1-c56 | 24.92% | 2.63% | 1.72% | 0.30% |
| Bxb1-c57 | 47.79% | 0.38% | 25.21% | 1.07% |
| Bxb1-c58 | 47.36% | 0.68% | 24.71% | 1.37% |
| Bxb1-c59 | 51.57% | 0.38% | 23.59% | 0.45% |
| Bxb1-c60 | 47.67% | 2.70% | 22.02% | 1.68% |
| Bxb1-c61 | 50.78% | 3.08% | 26.73% | 1.82% |
| Bxb1-c62 | 45.81% | 2.34% | 19.16% | 1.77% |
| Bxb1-c26 | 44.72% | 1.59% | 23.23% | 1.33% |
| Bxb1-c63 | 23.13% | 1.47% | 2.53% | 0.14% |
| Bxb1-c64 | 45.74% | 0.55% | 23.05% | 0.40% |
| Bxb1-c65 | 46.96% | 0.48% | 24.99% | 0.38% |
| Bxb1-c66 | 44.30% | 2.64% | 18.19% | 2.53% |
| Bxb1-c67 | 47.63% | 0.85% | 23.04% | 0.80% |
| Bxb1-c68 | 43.37% | 1.94% | 17.42% | 1.63% |
| Bxb1-c69 | 47.23% | 0.31% | 16.53% | 1.05% |

TABLE 19

| | |
|---|---|
| Prime Editing | Integration |

| | Editing | | Editing | |
|---|---|---|---|---|
| Integrase | Frequency | Stdev. | Frequency | Stdev. |
| No Integrase | 79.24% | 1.04% | 0.00% | 0.00% |
| WT Bxb1 | 79.17% | 0.07% | 25.85% | 0.29% |
| Bxb1-c22 | 87.65% | 0.67% | 50.61% | 1.92% |
| Bxb1-c2 | 88.07% | 2.33% | 46.18% | 4.19% |
| Bxb1-c26 | 75.74% | 0.55% | 35.22% | 1.61% |
| Bxb1-c71 | 81.38% | 0.61% | 37.69% | 0.03% |
| Bxb1-c72 | 85.05% | 0.18% | 44.18% | 1.95% |
| Bxb1-c73 | 72.33% | 0.35% | 32.08% | 0.41% |
| Bxb1-c74 | 76.91% | 2.32% | 34.79% | 1.70% |
| Bxb1-c75 | 67.89% | 3.40% | 25.16% | 2.68% |
| Bxb1-c76 | 69.08% | 2.28% | 25.26% | 1.07% |
| Bxb1-c77 | 65.78% | 1.04% | 25.70% | 0.31% |
| Bxb1-c78 | 66.80% | 1.81% | 26.65% | 2.02% |
| Bxb1-c79 | 74.70% | 1.77% | 33.37% | 1.80% |
| Bxb1-c80 | 87.37% | 1.06% | 51.83% | 1.83% |
| Bxb1-c81 | 85.81% | 0.04% | 48.94% | 1.29% |
| Bxb1-c82 | 82.12% | 1.06% | 44.28% | 0.53% |
| Bxb1-c83 | 86.04% | 1.78% | 46.53% | 0.44% |
| Bxb1-c84 | 81.58% | 0.96% | 38.90% | 0.32% |
| Bxb1-c85 | 81.61% | 1.81% | 36.61% | 0.35% |
| Bxb1-c86 | 68.62% | 0.37% | 22.77% | 0.77% |
| Bxb1-c87 | 70.83% | 0.22% | 17.80% | 0.12% |
| Bxb1-c88 | 73.89% | 1.14% | 23.31% | 0.63% |
| Bxb1-c89 | 69.51% | 0.31% | 21.08% | 0.64% |
| Bxb1-c90 | 83.90% | 0.69% | 46.54% | 1.72% |
| Bxb1-c91 | 68.32% | 1.58% | 27.84% | 0.48% |
| Bxb1-c92 | 78.52% | 1.04% | 42.75% | 0.20% |
| Bxb1-c93 | 73.92% | 1.58% | 32.51% | 0.87% |
| Bxb1-c94 | 82.06% | 3.61% | 42.67% | 3.28% |
| Bxb1-c95 | 78.00% | 2.56% | 36.61% | 0.83% |
| Bxb1-c96 | 74.01% | 1.88% | 25.84% | 1.38% |
| Bxb1-c97 | 72.68% | 1.11% | 11.24% | 0.16% |
| Bxb1-c98 | 83.42% | 1.93% | 33.31% | 0.05% |
| Bxb1-c99 | 82.67% | 1.95% | 29.75% | 1.38% |
| Bxb1-c100 | 75.64% | 2.78% | 20.30% | 0.53% |
| Bxb1-c101 | 78.73% | 1.28% | 33.54% | 0.20% |
| Bxb1-c103 | 75.27% | 2.89% | 36.69% | 2.84% |
| Bxb1-c105 | 72.70% | 0.37% | 33.25% | 0.56% |
| Bxb1-c107 | 70.06% | 3.06% | 25.34% | 1.83% |
| Bxb1-c108 | 73.37% | 1.33% | 30.09% | 0.61% |
| Bxb1-c109 | 83.45% | 0.23% | 43.86% | 1.17% |
| Bxb1-c110 | 84.97% | 0.84% | 45.47% | 0.66% |
| Bxb1-c111 | 79.40% | 1.13% | 33.49% | 2.61% |
| Bxb1-c113 | 80.24% | 1.37% | 41.60% | 2.32% |
| Bxb1-c114 | 81.91% | 1.16% | 31.52% | 0.45% |
| Bxb1-c116 | 83.41% | 1.98% | 41.69% | 0.07% |
| Bxb1-c117 | 82.20% | 0.73% | 45.68% | 0.56% |
| Bxb1-c118 | 83.11% | 0.76% | 48.18% | 2.50% |
| Bxb1-c119 | 82.72% | 0.92% | 49.67% | 0.55% |
| Bxb1-c120 | 80.14% | 1.65% | 45.48% | 2.48% |
| Bxb1-c121 | 60.79% | 2.14% | 8.57% | 0.03% |
| Bxb1-c122 | 74.89% | 2.82% | 33.19% | 0.48% |
| Bxb1-c123 | 85.72% | 2.20% | 44.23% | 3.63% |
| Bxb1-c124 | 81.55% | 0.13% | 44.94% | 0.84% |
| Bxb1-c125 | 82.44% | 1.38% | 41.34% | 0.80% |
| Bxb1-c126 | 80.87% | 2.55% | 44.45% | 2.57% |
| Bxb1-c127 | 84.60% | 2.55% | 48.58% | 0.88% |
| Bxb1-c128 | 84.93% | 1.12% | 46.44% | 2.28% |
| Bxb1-c129 | 84.29% | 1.76% | 44.91% | 3.18% |
| Bxb1-c130 | 84.49% | 1.19% | 44.77% | 2.61% |
| Bxb1-c131 | 82.52% | 0.20% | 47.52% | 1.06% |
| Bxb1-c132 | 57.30% | 0.86% | 11.07% | 0.28% |
| Bxb1-c133 | 66.87% | 0.43% | 24.46% | 0.56% |
| Bxb1-c134 | 83.49% | 0.23% | 43.57% | 1.16% |
| Bxb1-c135 | 82.88% | 0.13% | 44.72% | 0.06% |
| Bxb1-c136 | 80.87% | 0.69% | 42.13% | 0.67% |
| Bxb1-c137 | 81.64% | 0.84% | 47.90% | 1.32% |
| Bxb1-c138 | 85.77% | 1.48% | 46.63% | 2.67% |
| Bxb1-c139 | 81.46% | 0.15% | 40.54% | 2.15% |
| Bxb1-c140 | 83.43% | 0.55% | 42.39% | 0.17% |
| Bxb1-c141 | 80.05% | 2.03% | 44.04% | 0.33% |
| Bxb1-c142 | 80.59% | 1.45% | 43.43% | 1.06% |
| Bxb1-c143 | 56.52% | 1.71% | 8.72% | 0.81% |
| Bxb1-c144 | 66.03% | 0.09% | 24.49% | 0.54% |
| Bxb1-c145 | 86.33% | 1.44% | 45.19% | 0.09% |
| Bxb1-c146 | 85.79% | 1.16% | 48.03% | 2.63% |
| Bxb1-c147 | 79.61% | 1.01% | 45.01% | 1.26% |
| Bxb1-c148 | 57.59% | 1.75% | 9.46% | 0.23% |
| Bxb1-c149 | 77.38% | 1.64% | 37.42% | 0.46% |
| Bxb1-c150 | 83.43% | 0.50% | 45.31% | 1.17% |
| Bxb1-c151 | 82.98% | 2.07% | 48.04% | 2.11% |
| Bxb1-c152 | 79.90% | 1.67% | 40.97% | 2.78% |
| Bxb1-c153 | 64.72% | 2.12% | 17.22% | 1.27% |
| Bxb1-c154 | 83.55% | 0.71% | 50.51% | 3.56% |
| Bxb1-c155 | 82.06% | 1.53% | 33.17% | 0.87% |
| Bxb1-c156 | 71.23% | 4.05% | 25.56% | 1.41% |
| Bxb1-c157 | 83.86% | 2.41% | 49.37% | 2.50% |
| Bxb1-c158 | 80.07% | 2.53% | 40.24% | 1.36% |
| Bxb1-c159 | 70.51% | 4.18% | 28.53% | 0.16% |
| Bxb1-c160 | 82.10% | 0.89% | 47.74% | 2.00% |
| Bxb1-c161 | 80.71% | 1.13% | 36.46% | 1.23% |
| Bxb1-c162 | 78.70% | 1.38% | 36.73% | 2.97% |

Additional amino acid substitutions were introduced at positions 87, 91, 95 and 98 of Bxb1 of SEQ ID NO:1. Prime editing efficiency and integration of these mutants were analyzed (Table 20).

TABLE 20

| | Prime Editing | | Integration | |
|---|---|---|---|---|
| | Editing | | Editing | |
| Integrase mutations | Frequency | Stdev. | Frequency | Stdev. |
| No Integrase | 70.57% | 57.85% | 0.01% | 0.01% |
| WT Bxb1 | 73.27% | 60.05% | 20.48% | 16.79% |
| I87L A369P E434G | 74.27% | 60.87% | 46.44% | 38.07% |
| I87D | 41.97% | 34.40% | 1.23% | 1.01% |
| I87K | 41.60% | 34.10% | 1.56% | 1.28% |
| I87H | 43.94% | 36.01% | 4.19% | 3.43% |
| I87Y | 39.06% | 32.02% | 1.93% | 1.58% |
| I87L | 75.71% | 62.06% | 45.44% | 37.24% |
| I87A | 69.71% | 57.14% | 41.93% | 34.37% |
| I87S | 61.76% | 50.62% | 29.38% | 24.08% |
| Q91E | 72.63% | 59.53% | 0.31% | 0.26% |
| Q91K | 68.04% | 55.77% | 0.02% | 0.01% |
| Q91Y | 69.42% | 56.90% | 0.04% | 0.03% |
| Q91L | 73.04% | 59.87% | 2.12% | 1.74% |
| Q91A | 70.04% | 57.41% | 0.00% | 0.00% |
| Q91S | 69.48% | 56.95% | 0.02% | 0.01% |
| H95D | 42.61% | 34.93% | 2.17% | 1.78% |
| H95K | 70.13% | 57.48% | 0.16% | 0.13% |
| H95Y | 73.54% | 60.28% | 37.62% | 30.84% |
| H95L | 67.35% | 55.21% | 2.57% | 2.11% |
| H95A | 74.70% | 61.23% | 1.84% | 1.51% |
| H95S | 69.82% | 57.23% | 0.54% | 0.45% |
| E98D | 76.67% | 62.85% | 18.48% | 15.15% |
| E98K | 76.11% | 62.39% | 3.16% | 2.59% |
| E98H | 75.89% | 62.21% | 4.55% | 3.73% |
| E98Y | 70.86% | 58.09% | 12.20% | 10.00% |
| E98L | 71.81% | 58.86% | 4.73% | 3.88% |
| E98A | 72.45% | 59.38% | 3.18% | 2.61% |
| E98S | 75.12% | 61.57% | 14.99% | 12.29% |

### Example 4: Combinations of Mutations of Bxb1

In some aspects, an evolved Bxb1 integrase may include a mutation (e.g., an amino acid substitution) at one or more of positions 187 to H100 of SEQ ID NO:1, or any position or range of positions therein. In certain aspects, an evolved Bxb1 integrase includes an amino acid substitution at position 187, H89, Q91, Q92, H95, E98, D99 and/or H100 of SEQ ID NO:1, e.g., I87L, I87V, I87A, I87S, I87D, I87K, I87H, I87Y, H89Y, H89G, Q91E, Q91K, Q91Y, Q91L, Q91A, Q91S, Q92H, H95Y, H95D, H95K, H95L, H95A, H95S, E98D, E98K, E98H, E98Y, E98L, E98A, E98S, D99N, and/or H100N.

In some aspects, an evolved Bxb1 integrase may include a mutation (e.g., an amino acid substitution) at one or more of positions K101 to P117 of SEQ ID NO:1, or any position or range of positions therein. In certain aspects, an evolved Bxb1 integrase includes an amino acid substitution at position V105, A110, and/or H111 of SEQ ID NO:1, e.g., V105A, A110T and/or H111P.

In some aspects, an evolved Bxb1 integrase may include a mutation (e.g., an amino acid substitution) at one or more of positions F118 to 1137 of SEQ ID NO:1, or any position or range of positions therein. In certain aspects, an evolved Bxb1 integrase includes an amino acid substitution at positions A119 and/or V122 of SEQ ID NO:1, e.g., A119S and/or V122M.

In some aspects, an evolved Bxb1 integrase may include a mutation (e.g., an amino acid substitution) at one or more of positions A3 to 17, V74 to V76, and/or L103 to V105 of SEQ ID NO:1, or any position or range of positions therein. In certain aspects, an evolved Bxb1 integrase includes an amino acid substitution, e.g., a conservative amino acid substitution, at position L4, V74, 175, V76, and/or V105 of SEQ ID NO:1, e.g., L4I, V74I, I75V, V76I, and/or V105A.

In some aspects, an evolved Bxb1 integrase may include a mutation (e.g., an amino acid substitution) at one or more of positions P19 to Q32, P59 to A66, and/or V80 to L83 of SEQ ID NO:1, or any position or range of positions therein. In certain aspects, an evolved Bxb1 integrase includes an amino acid substitution at position L61 and/or R63 of SEQ ID NO:1, e.g., L61F and/or R63K.

In some aspects, an evolved Bxb1 integrase may include a mutation (e.g., an amino acid substitution) at one or more of positions G39 to L44 of SEQ ID NO:1, or any position or range of positions therein. In certain aspects, an evolved Bxb1 integrase includes an amino acid substitution at position V40 and/or E42 of SEQ ID NO:1, e.g., V40I, V40A and/or E42K.

In some aspects, an evolved Bxb1 integrase may include a mutation (e.g., an amino acid substitution) at one or more of positions R33 to V38, F67 to D73, and/or S106 to P117 of SEQ ID NO:1, or any position or range of positions therein. In certain aspects, an evolved Bxb1 integrase includes an amino acid substitution at position G34, D36, F67, E69, Q70, V105, and/or S106, A110, and/or H111 of SEQ ID NO:1, e.g., G34D, D36A, D36N, F67S, E69A, Q70H, V105A, A110T and/or H111P.

In some aspects, an evolved Bxb1 integrase may include a mutation (e.g., an amino acid substitution) at one or more of positions A374 to G422 of SEQ ID NO:1, or any position or range of positions therein. In certain aspects, an evolved Bxb1 integrase includes an amino acid substitution at position V380, T388, A398, R409, A411, A414, A415 and/or R416 of SEQ ID NO:1, e.g., V380I, T388M, A398S, R409H, A411V, A414V, A415S, and/or R416K.

In some aspects, an evolved Bxb1 integrase may include a mutation (e.g., an amino acid substitution) at one or more of positions D359 to A374 and/or L423 to R438 of SEQ ID NO:1, or any position or range of positions therein. In certain aspects, an evolved Bxb1 integrase includes an amino acid substitution at position D359, A360, R362, A369, A425, R426, R433 and/or E434 of SEQ ID NO:1, e.g., D359A, D359N, A360T, A360V, R362K, V366I, A369E, A369P, A369T, A369S, A425T, R426K, R433K, and/or E434G.

What is claimed is:

1. An evolved Bxb1 integrase comprising an amino acid sequence that is at least 90% identical to the amino acid sequence of SEQ ID NO:1, wherein the amino acid sequence of the evolved Bxb1 integrase comprises one or more mutations and exhibits at least a 2-fold increase in integration of exogenous nucleic acids into a genomic target site as compared to wild-type Bxb1 integrase.

2. The evolved Bxb1 integrase of claim 1, wherein the one or more mutations is at position 4, 5, 18, 24, 34, 36, 40, 42, 46, 51, 61, 62, 63, 67, 69, 79, 85, 87, 88, 89, 90, 92, 95, 99, 100, 105, 106, 110, 111, 119, 122, 130, 133, 137, 140, 145, 153, 156, 160, 164, 166, 174, 175, 178, 179, 181, 187, 189, 191, 203, 209, 218, 223, 229, 231, 239, 248, 251, 254, 261, 264, 268, 272, 278, 280, 281, 282, 283, 285, 287, 288, 292, 295, 302, 306, 307, 311, 313, 319, 321, 328, 331, 332, 333, 334, 347, 353, 355, 359, 360, 361, 362, 369, 370, 375, 380, 388, 397, 398, 405, 409, 411, 414, 415, 416, 419, 425, 428, 434, 435, 444, 453, 461, 463, 466, 468, 476, 479, 480, 483, 484, 487, 488, 489, 494, 496, and/or 499.

3. The evolved Bxb1 integrase of claim 1 or claim 2, wherein the one or more mutations comprise L4I, V5V, S18S, E24E, G34D, D36A, V40I, V40A, V40V, E42K, V46V, D51D, D51N, L61F, A62A, R63K, F67S, E69A, E69E, R79R, R85R, I87L, I87V, R88R, H89G, L90L, Q92H, Q92Q, H95Y, D99N, H100N, V105A, S106S, A110A, H111P, A119S, V122M, A130A, E133E, I137I, R140R, A145T, K153K, G156G, P160P, L164L, T166I, L174L, V175V, P178P, V179A, V179I, R181K, V187I, V187V, H189N, Q191Q, H203Y, G209G, A218A, R223R, E229K, S231S, M239I, A248T, N251K, N251N, T254S, A261T, V264A, P268P, R272Q, L278L, A280T, A280A, E281E, L282L, V283V, T285A, R287R, A288V, A288T, P292P, P295P, L302L, V306V, C307C, A311V, K313R, K313K, R319K, R319G, H321P, H321N, S328S, F331S, P332H, K333K, H334R, A347E, V353I, D355N, D359N, D359D, A360T, E361E, R362K, A369P, A369E, A369T, A369S, G370G, V375V, V380I, T388M, R397R, A398S, A405A, R409H, A411V, A414A, A414V, A415S, R416K, E419E, A425T, A425A, S428S, E434G, T435T, R444L, T453I, R461R, T463I, V466M, V466V, G468D, G468G, F476F, L479I, Q480STOP, E483E, Q484K, R487R, del L488, G489G, R494S, R494R, H496N, and/or M499T.

4. The evolved Bxb1 integrase of any one of the preceding claims, wherein the one or more mutations comprise D36N, D51G, D51E, H89Y, H100H, V175A, R181R, H189Y, A261S, R287H, V306A, D359A, A360V, and/or V466L.

5. The evolved Bxb1 integrase of any one of the preceding claims, wherein the one or more mutations comprise I87D, I87K, I87H, I87Y, I87A, I87S, H95D, H95K, H95L, H95A, and/or H95S.

6. The evolved Bxb1 integrase of any one of the preceding claims, wherein the one or more mutations is at position 45, 49, 50, 57, 70, 74, 75, 76, 168, 194, 263, 341, 366, 387, 426, and/or 445.

7. The evolved Bxb1 integrase of any one of the preceding claims, wherein the one or more mutations comprise D45G, A49S, V50I, R57K, Q70H, V74I, I75V, V76I, V168I, N194D, L263I, A341D, V366I, L387L, R426K, and/or E445Q.

8. The evolved Bxb1 integrase of any one of the preceding claims, wherein the one or more mutations is at position 91 and/or 98.

9. The evolved Bxb1 integrase of any one of the preceding claims, wherein the one or more mutations comprise Q91E, Q91K, Q91Y, Q91L, Q91A, Q91S, E98D, E98K, E98H, E98Y, E98L, E98A, and/or E98S.

10. The evolved Bxb1 integrase of any one of the preceding claims, wherein the one or more mutations are selected from one or more mutations of Table 1, Table 13, Table 17, and Table 20.

11. An evolved PhiC31 integrase comprising an amino acid sequence that is at least 90% identical to the amino acid sequence of SEQ ID NO:2, wherein the amino acid sequence of the evolved PhiC31 integrase comprises at least one mutation, the at least one mutation being at position 1, 2, 12, 14, 18, 24, 32, 36, 41, 43, 44, 45, 49, 51, 55, 58, 74, 77, 96, 103, 107, 117, 153, 176, 196, 197, 199, 200, 228, 229, 230, 231, 235, 238, 240, 252, 254, 255, 260, 262, 264, 266, 269, 274, 278, 302, 319, 320, 322, 331, 333, 340, 344, 345, 346, 347, 351, 353, 355, 359, 362, 364, 378, 382, 393, 396, 397, 399, 406, 410, 424, 429, 431, 436, 438, 445, 448, 449, 450, 452, 457, 468, 472, 478, 498, 499, 501, 505, 512, 514, 516, 517, 520, 527, 535, 536, 549, 551, 552, 553, 563, 568, 580, 585, 586, 587, 590, 592, 594, 600, 603, 604, 609, 612, and/or 616.

12. The evolved PhiC31 integrase of claim 11, wherein the at least one mutation comprises M1E, M1V, D2V, D2M, S12N, E14G, S18N, A24A, D32A, D36A, V41I, R43R, D44A, G45G, R49R, V51M, S55S, P58P, I74I, E77E, R96R, I103I, S107S, V117V, I153I, I153V, E176D, N196N, K197K, A199T, H200H, H228Y, L229L, P230S, F231L, S235S, A238A, H240R, D252G, D254D, A255G, G260G, T262T, G264G, K266K, S269N, P274S, M278L, T302A, L319L, R320R, V322V, E331E, A333T, A333S, A333D, A340V, G344V, G344D, R345R, G346S, R347K, L351V, L351Q, R353R, Q355Q, S359S, D362N, D362G, L364M, E378K, K382K, V393V, S396N, S396R, A397T, G399G, N406S, A410T, I424I, G429S, E431V, L436L, W438R, G445G, W448R, E449D, A450D, E452K, R457R, L468L, E472D, R478R, A498A, L499L, L501L, G505V, G505G, G505S, E512K, E514E, A516T, E517E, K520K, F527F, P535L, P535P, T536A, D549D, R551R, V552V, F553F, V563V, T568T, A580V, A585A, K586Q, P587P, D590G, D592G, D594N, D594D, T600S, T600T, V603I, V603A, V603V, A604S, P609S, V612V, and/or A616V.

13. The evolved PhiC31 integrase of claim 10 or claim 12, wherein the evolved PhiC31 integrase comprises at least one of the following mutations: M1E, D2V, D32A, D36A, V41I, and/or D44A.

14. The evolved PhiC31 integrase of any one of claims 11-13, wherein the evolved PhiC31 integrase further comprises an amino terminal sequence comprising any one of SEQ ID NOs:3-12.

15. The evolved PhiC31 integrase of any one of claims 11-14, wherein the one or more mutations are selected from one or more mutations of Table 2 and Table 12.

16. An isolated nucleic acid encoding the evolved Bxb1 or PhiC31 integrase of any one of claims 1-15.

17. A vector comprising the isolated nucleic acid of claim 16.

18. A cell comprising the evolved Bxb1 or PhiC31 integrase of any one of claims 1-15, the isolated nucleic acid of claim 16 or the vector of claim 17.

19. The cell of claim 18, wherein the cell is a human cell.

20. A method of altering the sequence of a genome of a cell comprising contacting a cell with the evolved Bxb1 or PhiC31 integrase of any one of claims 1-14, wherein the cell comprises in its genome at least one integrase recognition sequence recognized by the evolved Bxb1 or PhiC31 integrase so that the evolved Bxb1 or PhiC31 integrase binds to the integrase recognition sequence and alters the sequence of the genome of the cell.

21. The method of claim 20, wherein the alteration of the genome comprises a transversion, transition, deletion, insertion, inversion, replacement, or chromosomal translocation.

22. The method of claim 21, wherein the insertion comprises integration of an exogenous nucleic acid sequence into the genome of the cell at a safe harbor locus.

23. The method of claim 22, wherein the exogenous nucleic acid sequence encodes a therapeutic protein.

24. The method of 20, wherein the alteration delays development or progression of a disease or disorder or reduces disease severity.


SEQUENCE LISTING (372 sequences)

SEQ ID NO: 1 - protein, 500 aa, unidentified [sequence omitted]

SEQ ID NO: 2 - protein, 605 aa, unidentified
  source 1..605: note = Streptomyces phage C31
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 3 - protein, 8 aa, synthetic construct
MIQGVVSG

SEQ ID NO: 4 - protein, 8 aa, synthetic construct
MIQEVVSG

SEQ ID NO: 5 - protein, 40 aa, synthetic construct
MTMITPSAQLTLTKGNKSWSSLVTAASVLEFATMIQGVAG

SEQ ID NO: 6 - protein, 41 aa, synthetic construct
MTMIYPFPAQLTLTKGNKSWSSLVTAASVLEFATMIQGVAG

SEQ ID NO: 7 - protein, 40 aa, synthetic construct
MTMITPSAQLTLTKSNKSWNSLVTAASVLEFATMIQGVAG

SEQ ID NO: 8 - protein, 40 aa, synthetic construct; identical to SEQ ID NO: 5

SEQ ID NO: 9 - protein, 40 aa, synthetic construct
MTMITPSAQLTLTKSNKSWSSLVTAASVLEFATMIQGVTG

SEQ ID NO: 10 - protein, 40 aa, synthetic construct
MTMITPSAQLTLTKDNKSWSSLVTAASVLEFATMIQGVAG

SEQ ID NO: 11 - protein, 40 aa, synthetic construct
MTMITPSAQLTLTKSNKSWSSLVTAASVLEFATMIQGVAG

SEQ ID NO: 12 - protein, 40 aa, synthetic construct; identical to SEQ ID NO: 11

SEQ ID NO: 13 - DNA, 38 bp, synthetic construct
  source 1..38: mol_type = other DNA
ggcttgtcgacgacggcggtctccgtcgtcaggatcat

SEQ ID NO: 14 - DNA, 48 bp, synthetic construct
  source 1..48: mol_type = other DNA
ggtttgtctggtcaaccaccgcggtctcagtggtgtacggtacaaacc

SEQ ID NO: 15 - DNA, 35 bp, synthetic construct
  source 1..35: mol_type = other DNA
gtgccagggcgtgcccttgggctccccgggcgcgt

SEQ ID NO: 16 - DNA, 42 bp, synthetic construct
  source 1..42: mol_type = other DNA
tgccccaactggggtaacctttgagttctctcagttgggggc

SEQ ID NO: 17 - DNA, 63 bp, synthetic construct
  source 1..63: mol_type = other DNA
tcgtcggcagcgtcagatgtgtataagagacaggatgacctaactgcttctcctcttgggaag

SEQ ID NO: 18 - DNA, 63 bp, synthetic construct
  source 1..63: mol_type = other DNA
tcgtcggcagcgtcagatgtgtataagagacagctgtctgtaactgcttctcctcttgggaag

SEQ ID NO: 19 - DNA, 63 bp, synthetic construct
  source 1..63: mol_type = other DNA
tcgtcggcagcgtcagatgtgtataagagacagcacacagtaactgcttctcctcttgggaag

SEQ ID NO: 20 - DNA, 63 bp, synthetic construct
  source 1..63: mol_type = other DNA
tcgtcggcagcgtcagatgtgtataagagacagaaccggttaactgcttctcctcttgggaag

SEQ ID NO: 21 - DNA, 63 bp, synthetic construct
  source 1..63: mol_type = other DNA
tcgtcggcagcgtcagatgtgtataagagacagtctctcagaactgcttctcctcttgggaag

SEQ ID NO: 22 - DNA, 63 bp, synthetic construct
  source 1..63: mol_type = other DNA
tcgtcggcagcgtcagatgtgtataagagacaggagtcactaactgcttctcctcttgggaag

SEQ ID NO: 23 - DNA, 63 bp, synthetic construct
  source 1..63: mol_type = other DNA
tcgtcggcagcgtcagatgtgtataagagacagagactgagaactgcttctcctcttgggaag

SEQ ID NO: 24 - DNA, 63 bp, synthetic construct
  source 1..63: mol_type = other DNA
tcgtcggcagcgtcagatgtgtataagagacagggaatacgaactgcttctcctcttgggaag

SEQ ID NO: 25 - DNA, 63 bp, synthetic construct
  source 1..63: mol_type = other DNA
tcgtcggcagcgtcagatgtgtataagagacagagacctgtaactgcttctcctcttgggaag

SEQ ID NO: 26 - DNA, 63 bp, synthetic construct
  source 1..63: mol_type = other DNA
tcgtcggcagcgtcagatgtgtataagagacagtaggcgttaactgcttctcctcttgggaag

SEQ ID NO: 27 - DNA, 63 bp, synthetic construct
  source 1..63: mol_type = other DNA
tcgtcggcagcgtcagatgtgtataagagacagtgacgactaactgcttctcctcttgggaag

SEQ ID NO: 28 - DNA, 63 bp, synthetic construct
  source 1..63: mol_type = other DNA
tcgtcggcagcgtcagatgtgtataagagacagagagactgaactgcttctcctcttgggaag

SEQ ID NO: 29 - DNA, 63 bp, synthetic construct
  source 1..63: mol_type = other DNA
tcgtcggcagcgtcagatgtgtataagagacagatatgccgaactgcttctcctcttgggaag

SEQ ID NO: 30 - DNA, 61 bp, synthetic construct
  source 1..61: mol_type = other DNA
tcgtcggcagcgtcagatgtgtataagagacagcctacgaacactaaggcaattggggtgc

SEQ ID NO: 31 - DNA, 61 bp, synthetic construct
  source 1..61: mol_type = other DNA
tcgtcggcagcgtcagatgtgtataagagacagcgtaggaacactaaggcaattggggtgc

SEQ ID NO: 32 - DNA, 56 bp, synthetic construct
  source 1..56: mol_type = other DNA
tcgtcggcagcgtcagatgtgtataagagacagctctgactgccaggacggggctg

SEQ ID NO: 33 - DNA, 56 bp, synthetic construct
  source 1..56: mol_type = other DNA
tcgtcggcagcgtcagatgtgtataagagacaggtcatcaggccaggacggggctg

SEQ ID NO: 34 - DNA, 74 bp, synthetic construct
  source 1..74: mol_type = other DNA
tcgtcggcagcgtcagatgtgtataagagacagtcactctgaaggagaatgacagacaatagtatatatgaaat

SEQ ID NO: 35 - DNA, 74 bp, synthetic construct
  source 1..74: mol_type = other DNA
tcgtcggcagcgtcagatgtgtataagagacagctgacagtaaggagaatgacagacaatagtatatatgaaat

SEQ ID NO: 36 - DNA, 74 bp, synthetic construct
  source 1..74: mol_type = other DNA
tcgtcggcagcgtcagatgtgtataagagacagagtgacagaaggagaatgacagacaatagtatatatgaaat

SEQ ID NO: 37 - DNA, 74 bp, synthetic construct
  source 1..74: mol_type = other DNA
tcgtcggcagcgtcagatgtgtataagagacagacctacctaaggagaatgacagacaatagtatatatgaaat

SEQ ID NO: 38 - DNA, 74 bp, synthetic construct
  source 1..74: mol_type = other DNA
tcgtcggcagcgtcagatgtgtataagagacagcatgtggtaaggagaatgacagacaatagtatatatgaaat

SEQ ID NO: 39 - DNA, 74 bp, synthetic construct
  source 1..74: mol_type = other DNA
tcgtcggcagcgtcagatgtgtataagagacagttcgtaggaaggagaatgacagacaatagtatatatgaaat

SEQ ID NO: 40 - DNA, 74 bp, synthetic construct
  source 1..74: mol_type = other DNA
tcgtcggcagcgtcagatgtgtataagagacaggtcttgagaaggagaatgacagacaatagtatatatgaaat

SEQ ID NO: 41 - DNA, 74 bp, synthetic construct
  source 1..74: mol_type = other DNA
tcgtcggcagcgtcagatgtgtataagagacagacagagtgaaggagaatgacagacaatagtatatatgaaat

SEQ ID NO: 42 - DNA, 65 bp, synthetic construct
  source 1..65: mol_type = other DNA
tcgtcggcagcgtcagatgtgtataagagacagttcgaacggtgaatgactaagctccatttccc

SEQ ID NO: 43 - DNA, 65 bp, synthetic construct
  source 1..65: mol_type = other DNA
tcgtcggcagcgtcagatgtgtataagagacaggatgaggtgtgaatgactaagctccatttccc

SEQ ID NO: 44 - DNA, 65 bp, synthetic construct
  source 1..65: mol_type = other DNA
tcgtcggcagcgtcagatgtgtataagagacagcctacgttgtgaatgactaagctccatttccc

SEQ ID NO: 45 - DNA, 65 bp, synthetic construct
  source 1..65: mol_type = other DNA
tcgtcggcagcgtcagatgtgtataagagacaggagttgaggtgaatgactaagctccatttccc

SEQ ID NO: 46 - DNA, 59 bp, synthetic construct
  source 1..59: mol_type = other DNA
tcgtcggcagcgtcagatgtgtataagagacagttcgaacgctccatttccctacccca

SEQ ID NO: 47 - DNA, 59 bp, synthetic construct
  source 1..59: mol_type = other DNA
tcgtcggcagcgtcagatgtgtataagagacaggatgaggtctccatttccctacccca

SEQ ID NO: 48 - DNA, 59 bp, synthetic construct
  source 1..59: mol_type = other DNA
tcgtcggcagcgtcagatgtgtataagagacagcctacgttctccatttccctacccca

SEQ ID NO: 49 - DNA, 59 bp, synthetic construct
  source 1..59: mol_type = other DNA
tcgtcggcagcgtcagatgtgtataagagacaggagttgagctccatttccctacccca

SEQ ID NO: 50 - DNA, 60 bp, synthetic construct
  source 1..60: mol_type = other DNA
tcgtcggcagcgtcagatgtgtataagagacagggttcgatgggagggaagcactggttt

SEQ ID NO: 51 - DNA, 60 bp, synthetic construct
  source 1..60: mol_type = other DNA
tcgtcggcagcgtcagatgtgtataagagacagatcgttgggggagggaagcactggttt

SEQ ID NO: 52 - DNA, 64 bp, synthetic construct
  source 1..64: mol_type = other DNA
tcgtcggcagcgtcagatgtgtataagagacagagactctggagagggagaaagctagtgctat

SEQ ID NO: 53 - DNA, 64 bp, synthetic construct
  source 1..64: mol_type = other DNA
tcgtcggcagcgtcagatgtgtataagagacagcagtactggagagggagaaagctagtgctat

SEQ ID NO: 54 - DNA, 60 bp, synthetic construct
  source 1..60: mol_type = other DNA
gtctcgtgggctcggagatgtgtataagagacagcatccaagcccccatttcctggagcc

SEQ ID NO: 55 - DNA, 60 bp, synthetic construct
  source 1..60: mol_type = other DNA
gtctcgtgggctcggagatgtgtataagagacagcgttggatcccccatttcctggagcc

SEQ ID NO: 56 - DNA, 60 bp, synthetic construct
  source 1..60: mol_type = other DNA
gtctcgtgggctcggagatgtgtataagagacaggatctggtcccccatttcctggagcc

SEQ ID NO: 57 - DNA, 60 bp, synthetic construct
  source 1..60: mol_type = other DNA
gtctcgtgggctcggagatgtgtataagagacaggttccttgcccccatttcctggagcc

SEQ ID NO: 58 - DNA, 60 bp, synthetic construct
  source 1..60: mol_type = other DNA
gtctcgtgggctcggagatgtgtataagagacagctagtggtcccccatttcctggagcc

SEQ ID NO: 59 - DNA, 60 bp, synthetic construct
  source 1..60: mol_type = other DNA
gtctcgtgggctcggagatgtgtataagagacagcagtcagtcccccatttcctggagcc

SEQ ID NO: 60 - DNA, 60 bp, synthetic construct
  source 1..60: mol_type = other DNA
gtctcgtgggctcggagatgtgtataagagacaggtgtactgcccccatttcctggagcc

SEQ ID NO: 61 - DNA, 60 bp, synthetic construct
  source 1..60: mol_type = other DNA
gtctcgtgggctcggagatgtgtataagagacaggttgtcctcccccatttcctggagcc

SEQ ID NO: 62 - DNA, 60 bp, synthetic construct
  source 1..60: mol_type = other DNA
gtctcgtgggctcggagatgtgtataagagacagaccatggtcccccatttcctggagcc

SEQ ID NO: 63 - DNA, 60 bp, synthetic construct
  source 1..60: mol_type = other DNA
gtctcgtgggctcggagatgtgtataagagacagcacaactgcccccatttcctggagcc

SEQ ID NO: 64 - DNA, 63 bp, synthetic construct; identical to SEQ ID NO: 17
  source 1..63: mol_type = other DNA

SEQ ID NO: 65 - DNA, 63 bp, synthetic construct; identical to SEQ ID NO: 18
  source 1..63: mol_type = other DNA

SEQ ID NO: 66 - DNA, 63 bp, synthetic construct; identical to SEQ ID NO: 19
  source 1..63: mol_type = other DNA

SEQ ID NO: 67 - DNA, 63 bp, synthetic construct; identical to SEQ ID NO: 20
  source 1..63: mol_type = other DNA

SEQ ID NO: 68 - DNA, 63 bp, synthetic construct; identical to SEQ ID NO: 21
  source 1..63: mol_type = other DNA

SEQ ID NO: 69 - DNA, 63 bp, synthetic construct; identical to SEQ ID NO: 22
  source 1..63: mol_type = other DNA

SEQ ID NO: 70 - DNA, 63 bp, synthetic construct; identical to SEQ ID NO: 23
  source 1..63: mol_type = other DNA

SEQ ID NO: 71 - DNA, 63 bp, synthetic construct; identical to SEQ ID NO: 24
  source 1..63: mol_type = other DNA

SEQ ID NO: 72 - DNA, 63 bp, synthetic construct; identical to SEQ ID NO: 25
  source 1..63: mol_type = other DNA

SEQ ID NO: 73 - DNA, 63 bp, synthetic construct; identical to SEQ ID NO: 26
  source 1..63: mol_type = other DNA

SEQ ID NO: 74 - DNA, 63 bp, synthetic construct; identical to SEQ ID NO: 27
  source 1..63: mol_type = other DNA

SEQ ID NO: 75 - DNA, 63 bp, synthetic construct; identical to SEQ ID NO: 28
  source 1..63: mol_type = other DNA

SEQ ID NO: 76 - DNA, 63 bp, synthetic construct; identical to SEQ ID NO: 29
  source 1..63: mol_type = other DNA

SEQ ID NO: 77 - DNA, 61 bp, synthetic construct; identical to SEQ ID NO: 30
  source 1..61: mol_type = other DNA

SEQ ID NO: 78 - DNA, 61 bp, synthetic construct; identical to SEQ ID NO: 31
  source 1..61: mol_type = other DNA

SEQ ID NO: 79 - DNA, 56 bp, synthetic construct; identical to SEQ ID NO: 32
  source 1..56: mol_type = other DNA

SEQ ID NO: 80 - DNA, 56 bp, synthetic construct; identical to SEQ ID NO: 33
  source 1..56: mol_type = other DNA

SEQ ID NO: 81 - DNA, 74 bp, synthetic construct; identical to SEQ ID NO: 34
  source 1..74: mol_type = other DNA

SEQ ID NO: 82 - DNA, 74 bp, synthetic construct; identical to SEQ ID NO: 35
  source 1..74: mol_type = other DNA

SEQ ID NO: 83 - DNA, 74 bp, synthetic construct; identical to SEQ ID NO: 36
  source 1..74: mol_type = other DNA

SEQ ID NO: 84 - DNA, 74 bp, synthetic construct; identical to SEQ ID NO: 37
  source 1..74: mol_type = other DNA

SEQ ID NO: 85 - DNA, 74 bp, synthetic construct; identical to SEQ ID NO: 38
  source 1..74: mol_type = other DNA

SEQ ID NO: 86 - DNA, 74 bp, synthetic construct; identical to SEQ ID NO: 39
  source 1..74: mol_type = other DNA

SEQ ID NO: 87 - DNA, 74 bp, synthetic construct; identical to SEQ ID NO: 40
  source 1..74: mol_type = other DNA

SEQ ID NO: 88 - DNA, 74 bp, synthetic construct; identical to SEQ ID NO: 41
  source 1..74: mol_type = other DNA

SEQ ID NO: 89 - DNA, 65 bp, synthetic construct; identical to SEQ ID NO: 42
  source 1..65: mol_type = other DNA

SEQ ID NO: 90 - DNA, 65 bp, synthetic construct; identical to SEQ ID NO: 43
  source 1..65: mol_type = other DNA

SEQ ID NO: 91 - DNA, 65 bp, synthetic construct; identical to SEQ ID NO: 44
  source 1..65: mol_type = other DNA

SEQ ID NO: 92 - DNA, 65 bp, synthetic construct; identical to SEQ ID NO: 45
  source 1..65: mol_type = other DNA

SEQ ID NO: 93 - DNA, 59 bp, synthetic construct; identical to SEQ ID NO: 46
  source 1..59: mol_type = other DNA

SEQ ID NO: 94 - DNA, 59 bp, synthetic construct; identical to SEQ ID NO: 47
  source 1..59: mol_type = other DNA

SEQ ID NO: 95 - DNA, 59 bp, synthetic construct; identical to SEQ ID NO: 48
  source 1..59: mol_type = other DNA

SEQ ID NO: 96 - DNA, 59 bp, synthetic construct; identical to SEQ ID NO: 49
  source 1..59: mol_type = other DNA

SEQ ID NO: 97 - DNA, 60 bp, synthetic construct; identical to SEQ ID NO: 50
  source 1..60: mol_type = other DNA

SEQ ID NO: 98 - DNA, 60 bp, synthetic construct; identical to SEQ ID NO: 51
  source 1..60: mol_type = other DNA

SEQ ID NO: 99 - DNA, 64 bp, synthetic construct; identical to SEQ ID NO: 52
  source 1..64: mol_type = other DNA

SEQ ID NO: 100 - DNA, 64 bp, synthetic construct; identical to SEQ ID NO: 53
  source 1..64: mol_type = other DNA

SEQ ID NO: 101 - DNA, 60 bp, synthetic construct; identical to SEQ ID NO: 54
  source 1..60: mol_type = other DNA

SEQ ID NO: 102 - DNA, 60 bp, synthetic construct; identical to SEQ ID NO: 55
  source 1..60: mol_type = other DNA

SEQ ID NO: 103 - DNA, 60 bp, synthetic construct; identical to SEQ ID NO: 56
  source 1..60: mol_type = other DNA

SEQ ID NO: 104 - DNA, 60 bp, synthetic construct; identical to SEQ ID NO: 57
  source 1..60: mol_type = other DNA

SEQ ID NO: 105 - DNA, 60 bp, synthetic construct; identical to SEQ ID NO: 58
  source 1..60: mol_type = other DNA

SEQ ID NO: 106 - DNA, 60 bp, synthetic construct; identical to SEQ ID NO: 59
  source 1..60: mol_type = other DNA

SEQ ID NO: 107 - DNA, 60 bp, synthetic construct; identical to SEQ ID NO: 60
  source 1..60: mol_type = other DNA

SEQ ID NO: 108 - DNA, 60 bp, synthetic construct; identical to SEQ ID NO: 61
  source 1..60: mol_type = other DNA

SEQ ID NO: 109 - DNA, 60 bp, synthetic construct; identical to SEQ ID NO: 62
  source 1..60: mol_type = other DNA

SEQ ID NO: 110 - DNA, 60 bp, synthetic construct; identical to SEQ ID NO: 63
  source 1..60: mol_type = other DNA

SEQ ID NO: 111 - DNA, 60 bp, synthetic construct
  source 1..60: mol_type = other DNA
gtctcgtgggctcggagatgtgtataagagacagctctctctcccccatttcctggagcc

SEQ ID NO: 112 - DNA, 60 bp, synthetic construct
  source 1..60: mol_type = other DNA
gtctcgtgggctcggagatgtgtataagagacagtaggaacgcccccatttcctggagcc

SEQ ID NO: 113 - DNA, 60 bp, synthetic construct
  source 1..60: mol_type = other DNA
gtctcgtgggctcggagatgtgtataagagacagtctccagtcccccatttcctggagcc

SEQ ID NO: 114 - DNA, 65 bp, synthetic construct
  source 1..65: mol_type = other DNA
gtctcgtgggctcggagatgtgtataagagacagagtcgactgcctagttttagcactgaaaccc

SEQ ID NO: 115 - DNA, 65 bp, synthetic construct
  source 1..65: mol_type = other DNA
gtctcgtgggctcggagatgtgtataagagacagaacgtagggcctagttttagcactgaaaccc

SEQ ID NO: 116 - DNA, 58 bp, synthetic construct
  source 1..58: mol_type = other DNA
gtctcgtgggctcggagatgtgtataagagacagactggactggccaccctgcgctac

SEQ ID NO: 117 - DNA, 58 bp, synthetic construct
  source 1..58: mol_type = other DNA
gtctcgtgggctcggagatgtgtataagagacagactcacagggccaccctgcgctac

SEQ ID NO: 118 - DNA, 68 bp, synthetic construct
  source 1..68: mol_type = other DNA
gtctcgtgggctcggagatgtgtataagagacagctacgtagcccaggttacatttggttgatatgtc

SEQ ID NO: 119 - DNA, 68 bp, synthetic construct
  source 1..68: mol_type = other DNA
gtctcgtgggctcggagatgtgtataagagacagttccggttcccaggttacatttggttgatatgtc

SEQ ID NO: 120 - DNA, 66 bp, synthetic construct
  source 1..66: mol_type = other DNA
gtctcgtgggctcggagatgtgtataagagacaggacatgagcaggtattttaaatggtccccagg

SEQ ID NO: 121 - DNA, 66 bp, synthetic construct
  source 1..66: mol_type = other DNA
gtctcgtgggctcggagatgtgtataagagacagcaaccatgcaggtattttaaatggtccccagg

SEQ ID NO: 122 - DNA, 66 bp, synthetic construct
  source 1..66: mol_type = other DNA
gtctcgtgggctcggagatgtgtataagagacagacacctctcaggtattttaaatggtccccagg

SEQ ID NO: 123 - DNA, 66 bp, synthetic construct
  source 1..66: mol_type = other DNA
gtctcgtgggctcggagatgtgtataagagacagctcaacagcaggtattttaaatggtccccagg

SEQ ID NO: 124 - DNA, 66 bp, synthetic construct
  source 1..66: mol_type = other DNA
gtctcgtgggctcggagatgtgtataagagacagacgttggtcaggtattttaaatggtccccagg

SEQ ID NO: 125 - DNA, 66 bp, synthetic construct
  source 1..66: mol_type = other DNA
gtctcgtgggctcggagatgtgtataagagacagaacgatggcaggtattttaaatggtccccagg

SEQ ID NO: 126 - DNA, 64 bp, synthetic construct
  source 1..64: mol_type = other DNA
gtctcgtgggctcggagatgtgtataagagacagactgtgtgagaagaggtcagaaagccagtc

SEQ ID NO: 127 - DNA, 64 bp, synthetic construct
  source 1..64: mol_type = other DNA
gtctcgtgggctcggagatgtgtataagagacagtggtgaagagaagaggtcagaaagccagtc

SEQ ID NO: 128 - DNA, 64 bp, synthetic construct
  source 1..64: mol_type = other DNA
gtctcgtgggctcggagatgtgtataagagacagcttctcctagaagaggtcagaaagccagtc

SEQ ID NO: 129 - DNA, 64 bp, synthetic construct
  source 1..64: mol_type = other DNA
gtctcgtgggctcggagatgtgtataagagacaggagtgtctagaagaggtcagaaagccagtc

SEQ ID NO: 130 - DNA, 60 bp, synthetic construct
  source 1..60: mol_type = other DNA
gtctcgtgggctcggagatgtgtataagagacagactgtgtggtcagaaagccagtcgcg

SEQ ID NO: 131 - DNA, 60 bp, synthetic construct
  source 1..60: mol_type = other DNA
gtctcgtgggctcggagatgtgtataagagacagtggtgaaggtcagaaagccagtcgcg

SEQ ID NO: 132 - DNA, 60 bp, synthetic construct
  source 1..60: mol_type = other DNA
gtctcgtgggctcggagatgtgtataagagacagcttctcctgtcagaaagccagtcgcg

SEQ ID NO: 133 - DNA, 60 bp, synthetic construct
  source 1..60: mol_type = other DNA
gtctcgtgggctcggagatgtgtataagagacaggagtgtctgtcagaaagccagtcgcg

SEQ ID NO: 134 - DNA, 68 bp, synthetic construct
  source 1..68: mol_type = other DNA
gtctcgtgggctcggagatgtgtataagagacagtctcagagggttattgtaataagggtggggtagg

SEQ ID NO: 135 - DNA, 68 bp, synthetic construct
  source 1..68: mol_type = other DNA
gtctcgtgggctcggagatgtgtataagagacagcagttcagggttattgtaataagggtggggtagg

SEQ ID NO: 136 - DNA, 68 bp, synthetic construct
  source 1..68: mol_type = other DNA
gtctcgtgggctcggagatgtgtataagagacagagtgcactcctgagttttataccatttgagaccc

SEQ ID NO: 137 - DNA, 68 bp, synthetic construct
  source 1..68: mol_type = other DNA
gtctcgtgggctcggagatgtgtataagagacagacctaggtcctgagttttataccatttgagaccc

SEQ ID NO: 138 - DNA, 65 bp, synthetic construct; identical to SEQ ID NO: 42
  source 1..65: mol_type = other DNA

SEQ ID NO: 139 - DNA, 65 bp, synthetic construct; identical to SEQ ID NO: 43
  source 1..65: mol_type = other DNA

SEQ ID NO: 140 - DNA, 65 bp, synthetic construct; identical to SEQ ID NO: 44
  source 1..65: mol_type = other DNA

SEQ ID NO: 141 - DNA, 65 bp, synthetic construct; identical to SEQ ID NO: 45
  source 1..65: mol_type = other DNA

SEQ ID NO: 142 - DNA, 59 bp, synthetic construct; identical to SEQ ID NO: 46
  source 1..59: mol_type = other DNA

SEQ ID NO: 143 - DNA, 59 bp, synthetic construct; identical to SEQ ID NO: 47
  source 1..59: mol_type = other DNA

SEQ ID NO: 144 - DNA, 59 bp, synthetic construct; identical to SEQ ID NO: 48
  source 1..59: mol_type = other DNA

SEQ ID NO: 145 - DNA, 59 bp, synthetic construct; identical to SEQ ID NO: 49
  source 1..59: mol_type = other DNA

SEQ ID NO: 146 - DNA, 64 bp, synthetic construct; identical to SEQ ID NO: 126
  source 1..64: mol_type = other DNA

SEQ ID NO: 147 - DNA, 64 bp, synthetic construct; identical to SEQ ID NO: 127
  source 1..64: mol_type = other DNA

SEQ ID NO: 148 - DNA, 64 bp, synthetic construct; identical to SEQ ID NO: 128
  source 1..64: mol_type = other DNA

SEQ ID NO: 149 - DNA, 64 bp, synthetic construct; identical to SEQ ID NO: 129
  source 1..64: mol_type = other DNA

SEQ ID NO: 150 - DNA, 60 bp, synthetic construct; identical to SEQ ID NO: 130
  source 1..60: mol_type = other DNA

SEQ ID NO: 151 - DNA, 60 bp, synthetic construct; identical to SEQ ID NO: 131
  source 1..60: mol_type = other DNA

SEQ ID NO: 152 - DNA, 60 bp, synthetic construct; identical to SEQ ID NO: 132
  source 1..60: mol_type = other DNA

SEQ ID NO: 153 - DNA, 60 bp, synthetic construct; identical to SEQ ID NO: 133
  source 1..60: mol_type = other DNA

SEQ ID NO: 154 - DNA, 24 bp, synthetic construct
  source 1..24: mol_type = other DNA
gtgaatgactaagctccatttccc

SEQ ID NO: 155 - DNA, 22 bp, synthetic construct
  source 1..22: mol_type = other DNA
agaagaggtcagaaagccagtc

SEQ ID NO: 156 - DNA, 18 bp, synthetic construct
  source 1..18: mol_type = other DNA
ctccatttccctacccca

SEQ ID NO: 157 - DNA, 18 bp, synthetic construct
  source 1..18: mol_type = other DNA
gtcagaaagccagtcgcg

SEQ ID NO: 158 - DNA, 21 bp, synthetic construct
  source 1..21: mol_type = other DNA
ccctaccccacccttattaca

SEQ ID NO: 159 - DNA, 20 bp, synthetic construct
  source 1..20: mol_type = other DNA
gaggtcagaaagccagtcgc

SEQ ID NO: 160 - DNA, 259 bp, synthetic construct
  source 1..259: mol_type = other DNA
cactaaggcaattggggtgcaggaatgggggcagggtaccagcctcaccaagtggttgataaacccacgtggggtaccctaagaacttgggaacagccacagcaggggggcgatgcttggggacctgcctggagaaggatgcaggacgagaaacacagccccaggtggagaaactggccgggaatcaagagtcacccagagacagtgaccaaccatccctgttttcctaggactgagggtttcagtgctaaaactaggc

SEQ ID NO: 161 - DNA, 196 bp, synthetic construct
  source 1..196: mol_type = other DNA
cactaaggcaattggggtgcaggaatgggggcagggtaccagcctcaccaagtggttgataaacccgtgccagggcgtgcccttgggctccccgggcgcgtgtggagaaactggccgggaatcaagagtcacccagagacagtgaccaaccatccctgttttcctaggactgagggtttcagtgctaaaactaggc

SEQ ID NO: 162 - DNA, 202 bp, synthetic construct
  source 1..202: mol_type = other DNA
cactaaggcaattggggtgcaggaatgggggcagggtaccagcctcaccaagtggttgataaacccgcccccaactgagagaactcaaaggttaccccagttggggcgtggagaaactggccgggaatcaagagtcacccagagacagtgaccaaccatccctgttttcctaggactgagggtttcagtgctaaaactaggc

SEQ ID NO: 163 - DNA, 251 bp, synthetic construct
  source 1..251: mol_type = other DNA
gccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggcc

SEQ ID NO: 164 - DNA, 184 bp, synthetic construct
  source 1..184: mol_type = other DNA
gccaggacggggctggctactggccttatctcacaggtaaaactgacgcagtgccagggcgtgcccttgggctccccgggcgcgtgggaagtgtaaggaagctgcagcaccaggatcagtgaaacgcaccagacagccgcgtcagagcagctcaggttctgggagagggtagcgcagggtggcc

SEQ ID NO: 165 - DNA, 190 bp, synthetic construct
  source 1..190: mol_type = other DNA
gccaggacggggctggctactggccttatctcacaggtaaaactgacgcagcccccaactgagagaactcaaaggttaccccagttggggcgggaagtgtaaggaagctgcagcaccaggatcagtgaaacgcaccagacagccgcgtcagagcagctcaggttctgggagagggtagcgcagggtggcc

SEQ ID NO: 166 - DNA, 256 bp, synthetic construct
  source 1..256: mol_type = other DNA
aactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatggggg

SEQ ID NO: 167 - DNA, 182 bp, synthetic construct
  source 1..182: mol_type = other DNA
aactgcttctcctcttgggaagtgtaaggaagctgcagcaccaggatcagtgaaacgcaccaggtttgtctggtcaaccaccgcggtctcagtggtgtacggtacaaacctcctctctggctccatcgtaagcaaaccttagaggttctggcaaggagagagatggctccaggaaatggggg

SEQ ID NO: 168 - DNA, 168 bp, synthetic construct
  source 1..168: mol_type = other DNA
aactgcttctcctcttgggaagtgtaaggaagctgcagcaccaggatcagtgaaacgcaccgtgccagggcgtgcccttgggctccccgggcgcgttcctctctggctccatcgtaagcaaaccttagaggttctggcaaggagagagatggctccaggaaatggggg

SEQ ID NO: 169 - DNA, 174 bp, synthetic construct
  source 1..174: mol_type = other DNA
aactgcttctcctcttgggaagtgtaaggaagctgcagcaccaggatcagtgaaacgcaccgcccccaactgagagaactcaaaggttaccccagttggggctcctctctggctccatcgtaagcaaaccttagaggttctggcaaggagagagatggctccaggaaatggggg

SEQ ID NO: 170 - DNA, 256 bp, synthetic construct; identical to SEQ ID NO: 166
  source 1..256: mol_type = other DNA

SEQ ID NO: 171 - DNA, 215 bp, synthetic construct
  source 1..215: mol_type = other DNA
aactgcttctcctcttgggaagtgtaaggaagctgcagcaccaggatcagtgaaacgcaccagacagccgcgtcagagcagctcaggttctgggaggtttgtctggtcaaccaccgcggtctcagtggtgtacggtacaaacctcctctctggctccatcgtaagcaaaccttagaggttctggcaaggagagagatggctccaggaaatggggg

SEQ ID NO: 172 - DNA, 201 bp, synthetic construct
  source 1..201: mol_type = other DNA
aactgcttctcctcttgggaagtgtaaggaagctgcagcaccaggatcagtgaaacgcaccagacagccgcgtcagagcagctcaggttctggggtgccagggcgtgcccttgggctccccgggcgcgttcctctctggctccatcgtaagcaaaccttagaggttctggcaaggagagagatggctccaggaaatggggg

SEQ ID NO: 173 - DNA, 207 bp, synthetic construct
  source 1..207: mol_type = other DNA
aactgcttctcctcttgggaagtgtaaggaagctgcagcaccaggatcagtgaaacgcaccagacagccgcgtcagagcagctcaggttctggggcccccaactgagagaactcaaaggttaccccagttggggctcctctctggctccatcgtaagcaaaccttagaggttctggcaaggagagagatggctccaggaaatggggg

SEQ ID NO: 174 - DNA, 249 bp, synthetic construct
  source 1..249: mol_type = other DNA
gggagggaagcactggtttctcaagcaaaagctaaaatttttctattaagatttaacctgatgctacactttggtggtgcagcaagggtctcaaatggtataaaactcaggtgatcatgctttatgtctgtctctagaaaaatgctccaaaaatgataagtagtgataatccgcagtctcgttgcataaaatcagccccaggtgaatgactaagctccatttccctaccccacccttattacaataacc

SEQ ID NO: 175 - DNA, 188 bp, synthetic construct
  source 1..188: mol_type = other DNA
gggagggaagcactggtttctcaagcaaaagctaaaatttttctattaagatttaacctgatgctacactttggtggtgcagcaagggtctcaaatggtataaaagtgccagggcgtgcccttgggctccccgggcgcgtgtgaatgactaagctccatttccctaccccacccttattacaataacc

SEQ ID NO: 176 - DNA, 194 bp, synthetic construct
  source 1..194: mol_type = other DNA
gggagggaagcactggtttctcaagcaaaagctaaaatttttctattaagatttaacctgatgctacactttggtggtgcagcaagggtctcaaatggtataaaagcccccaactgagagaactcaaaggttaccccagttggggcgtgaatgactaagctccatttccctaccccacccttattacaataacc

SEQ ID NO: 177 - DNA, 251 bp, synthetic construct
  source 1..251: mol_type = other DNA
gagagggagaaagctagtgctatgtctgaatactagaggagcaagtacaacaaatggaaaatgggatcaagtatgagtgagagttgctaagatgcctggtagggatgcaaaggggtagagagcctggggagagagggtgagggagggaagcactggtttctcaagcaaaagctaaaatttttctattaagatttaacctgatgctacactttggtggtgcagcaagggtctcaaatggtataaaactcagg

SEQ ID NO: 178 - DNA, 192 bp, synthetic construct
  source 1..192: mol_type = other DNA
gagagggagaaagctagtgctatgtctgaatactagaggagcaagtacaacaaatggaaaatgggatcaagtatgagtgagagttgctaagatgcctggtagggatgcgtgccagggcgtgcccttgggctccccgggcgcgtgctacactttggtggtgcagcaagggtctcaaatggtataaaactcagg

SEQ ID NO: 179 - DNA, 198 bp, synthetic construct
  source 1..198: mol_type = other DNA
gagagggagaaagctagtgctatgtctgaatactagaggagcaagtacaacaaatggaaaatgggatcaagtatgagtgagagttgctaagatgcctggtagggatgcgcccccaactgagagaactcaaaggttaccccagttggggcgctacactttggtggtgcagcaagggtctcaaatggtataaaactcagg

SEQ ID NO: 180 - DNA, 261 bp, synthetic construct
  source 1..261: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagaatgtgggaaacttaggaagacacccagtttcttcaacaaaaaaattgcaaaaataaaaaggaacaaggagaacctatagattaaatgaggcattaaagacatatcaaccaaatgtaacctggg

SEQ ID NO: 181 - DNA, 151 bp, synthetic construct
  source 1..151: mol_type = other DNA
aaggagaatgacagacaatagtatatatgaaatattcttatcaaaagataagatctactctaattaaacctgtgccagggcgtgcccttgggctccccgggcgcgtgattaaatgaggcattaaagacatatcaaccaaatgtaacctggg

SEQ ID NO: 182 - DNA, 157 bp, synthetic construct
  source 1..157: mol_type = other DNA
aaggagaatgacagacaatagtatatatgaaatattcttatcaaaagataagatctactctaattaaacctgcccccaactgagagaactcaaaggttaccccagttggggcgattaaatgaggcattaaagacatatcaaccaaatgtaacctggg

SEQ ID NO: 183 - DNA, 279 bp, synthetic construct
  source 1..279: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagaatgtgggaaacttaggaagacacccagtttcttcaacaaaaaaattgcaaaaataaaaaggaacaaggagaacctatagattaaatgaggcattaaagacatatcaaccaaatgtaacctggggaccatttaaaatacctg

SEQ ID NO: 184 - DNA, 134 bp, synthetic construct
  source 1..134: mol_type = other DNA
aaggagaatgacagacaatagtatatatgaaatattcttatcaaaagataagatctactctaattaaacctgtgccagggcgtgcccttgggctccccgggcgcgtgtaacctggggaccatttaaaatacctg

SEQ ID NO: 185 - DNA, 140 bp, synthetic construct
  source 1..140: mol_type = other DNA
aaggagaatgacagacaatagtatatatgaaatattcttatcaaaagataagatctactctaattaaacctgcccccaactgagagaactcaaaggttaccccagttggggcgtaacctggggaccatttaaaatacctg

SEQ ID NO: 186 - DNA, 279 bp, synthetic construct; identical to SEQ ID NO: 183
  source 1..279: mol_type = other DNA

SEQ ID NO: 187 - DNA, 236 bp, synthetic construct
  source 1..236: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagagtgccagggcgtgcccttgggctccccgggcgcgtgattaaatgaggcattaaagacatatcaaccaaatgtaacctggggaccatttaaaatacctg

SEQ ID NO: 188 - DNA, 242 bp, synthetic construct
  source 1..242: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagagcccccaactgagagaactcaaaggttaccccagttggggcgattaaatgaggcattaaagacatatcaaccaaatgtaacctggggaccatttaaaatacctg

SEQ ID NO: 189 - DNA, 279 bp, synthetic construct; identical to SEQ ID NO: 183
  source 1..279: mol_type = other DNA

SEQ ID NO: 190 - DNA, 201 bp, synthetic construct
  source 1..201: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagagtgccagggcgtgcccttgggctccccgggcgcgtgtaacctggggaccatttaaaatacctg

SEQ ID NO: 191 - DNA, 207 bp, synthetic construct
  source 1..207: mol_type = other DNA
aaggagaatgacagacaatagtatatatgaaatattcttatcaaaagataagatctactctaattaaacctgtagggaaaaaagggacaaaggagcatgttaaacaatatcatgagaatagaatgagcaaactctagagcccccaactgagagaactcaaaggttaccccagttggggcgtaacctggggaccatttaaaatacctg

SEQ ID NO: 192 - DNA, 20 bp, synthetic construct
  source 1..20: mol_type = other DNA
gtttgtccaaactcagcggc

SEQ ID NO: 193 - DNA, 20 bp, synthetic construct
  source 1..20: mol_type = other DNA
gaaggtacgcctgcaggtac

SEQ ID NO: 194 - DNA, 20 bp, synthetic construct
  source 1..20: mol_type = other DNA
atccagcctccggactctag

SEQ ID NO: 195 - DNA, 16 bp, synthetic construct
  source 1..16: mol_type = other DNA
ccggggagcccaaggg

SEQ ID NO: 196 - DNA, 20 bp, synthetic construct
  source 1..20: mol_type = other DNA
gagttctctcagttgggggc

SEQ ID NO: 197 - DNA, 22 bp, synthetic construct
  source 1..22: mol_type = other DNA
ccaactggggtaacctttgagt

SEQ ID NO: 198 - DNA, 20 bp, synthetic construct
  source 1..20: mol_type = other DNA
tccgcagtctcgttgcataa

SEQ ID NO: 199 - DNA, 23 bp, synthetic construct
  source 1..23: mol_type = other DNA
acaacaaatggaaaatgggatca

SEQ ID NO: 200 - DNA, 20 bp, synthetic construct
  source 1..20: mol_type = other DNA
agaacgtgaactagggagga

SEQ ID NO: 201 - DNA, 25 bp, synthetic construct
  source 1..25: mol_type = other DNA
agcctctatattctaatccacttgt

SEQ ID NO: 202 - DNA, 20 bp, synthetic construct
  source 1..20: mol_type = other DNA
ctgtgcctagcctaagcctc

SEQ ID NO: 203 - DNA, 20 bp, synthetic construct
  source 1..20: mol_type = other DNA
tgctggaattacaggcgtga

SEQ ID NO: 204 - DNA, 23 bp, synthetic construct
  source 1..23: mol_type = other DNA
gcttctcctcttgggaagtgtaa

SEQ ID NO: 205 - DNA, 20 bp, synthetic construct
  source 1..20: mol_type = other DNA
aagggagttttccacacgga

SEQ ID NO: 206 - DNA, 20 bp, synthetic construct
  source 1..20: mol_type = other DNA
cactaaggcaattggggtgc

SEQ ID NO: 207 - DNA, 18 bp, synthetic construct
  source 1..18: mol_type = other DNA
gtcgacgacggcggtctc

SEQ ID NO: 208 - DNA, 20 bp, synthetic construct
  source 1..20: mol_type = other DNA
ctccgtcgtcaggatcatcc

SEQ ID NO: 209 - DNA, 19 bp, synthetic construct
  source 1..19: mol_type = other DNA
gtacaccactgagaccgcg

SEQ ID NO: 210 - DNA, 19 bp, synthetic construct
  source 1..19: mol_type = other DNA
ccgtgacctacatgctcgc

SEQ ID NO: 211 - DNA, 18 bp, synthetic construct
  source 1..18: mol_type = other DNA
ctcgaagggcgtatgcgc

SEQ ID NO: 212 - DNA, 33 bp, synthetic construct
  source 1..33: mol_type = other DNA
ccccaggtgaatgactaagctccatttccctac

SEQ ID NO: 213 - DNA, 20 bp, synthetic construct
  source 1..20: mol_type = other DNA
ggggagtgagcagctgtaag

SEQ ID NO: 214 - DNA, 33 bp, synthetic construct
  source 1..33: mol_type = other DNA
agtgagagttgctaagatgcctggtagggatgc

SEQ ID NO: 215 - DNA, 20 bp, synthetic construct
  source 1..20: mol_type = other DNA
tgctgcaccaccaaagtgta

SEQ ID NO: 216 - DNA, 33 bp, synthetic construct
  source 1..33: mol_type = other DNA
tagggccctgatatgggcacccaaatgtagctt

SEQ ID NO: 217 - DNA, 20 bp, synthetic construct
  source 1..20: mol_type = other DNA
aacatgctcctttgtccctt

SEQ ID NO: 218 - DNA, 28 bp, synthetic construct
  source 1..28: mol_type = other DNA
ctgcagcaccaggatcagtgaaacgcac

SEQ ID NO: 219 - DNA, 19 bp, synthetic construct
  source 1..19: mol_type = other DNA
gttctcagtggccaccctg

SEQ ID NO: 220 - DNA, 24 bp, synthetic construct
  source 1..24: mol_type = other DNA
cccctcctcaccacagccctgcca

SEQ ID NO: 221 - DNA, 22 bp, synthetic construct
  source 1..22: mol_type = other DNA
ggctcttcacctttctagtccc

SEQ ID NO: 222 - DNA, 33 bp, synthetic construct
  source 1..33: mol_type = other DNA
taccagcctcaccaagtggttgataaacccacg

SEQ ID NO: 223 - DNA, 20 bp, synthetic construct
  source 1..20: mol_type = other DNA
ctcgtcctgcatccttctcc

SEQ ID NO: 224 - DNA, 17 bp, synthetic construct
  source 1..17: mol_type = other DNA
acactgtgcccatctac

SEQ ID NO: 225 - DNA, 18 bp, synthetic construct
  source 1..18: mol_type = other DNA
aatgtcacgcacgatttc

SEQ ID NO: 226 - DNA, 19 bp, synthetic construct
  source 1..19: mol_type = other DNA
agatttggacctgcgagcg

SEQ ID NO: 227 - DNA, 20 bp, synthetic construct
  source 1..20: mol_type = other DNA
gagcggctgtctccacaagt

SEQ ID NO: 228 - DNA, 24 bp, synthetic construct
  source 1..24: mol_type = other DNA
gtactcttgtccatctgttctctg

SEQ ID NO: 229 - DNA, 22 bp, synthetic construct
  source 1..22: mol_type = other DNA
ggccgatattcagcccttaaac

SEQ ID NO: 230 - DNA, 33 bp, synthetic construct
  source 1..33: mol_type = other DNA
ccgtgacctacatgctcgaagggcgtatgcgcc

SEQ ID NO: 231 - DNA, 104 bp, synthetic construct
  source 1..104: mol_type = other DNA
gtaacgctcttcgagaaagcagattctcatatccatcttgagtcttctttctcgcaagacaacacgaaatagacacagtctcttccctagctgtacactgagcc

SEQ ID NO: 232 - DNA, 28 bp, synthetic construct
  source 1..28: mol_type = other DNA
ttctagagacagacataaagcatgatca

SEQ ID NO: 233 - DNA, 26 bp, synthetic construct
  source 1..26: mol_type = other DNA
ggaaataagttatcacaatgggaaat

SEQ ID NO: 234 - DNA, 22 bp, synthetic construct
  source 1..22: mol_type = other DNA
tcgcgattcttaaaaggagagg

SEQ ID NO: 235 - DNA, 24 bp, synthetic construct
  source 1..24: mol_type = other DNA
ccattcatattttgaaacaaaagg

SEQ ID NO: 236 - DNA, 23 bp, synthetic construct
  source 1..23: mol_type = other DNA
gcattgcactcctacatacaaca

SEQ ID NO: 237 - DNA, 20 bp, synthetic construct
  source 1..20: mol_type = other DNA
gctgtggttattcccagctc

SEQ ID NO: 238 - DNA, 22 bp, synthetic construct
  source 1..22: mol_type = other DNA
ctgggaacactggacaaaatcc

SEQ ID NO: 239 - DNA, 21 bp, synthetic construct
  source 1..21: mol_type = other DNA
ggaaagctttgacaagtggaa

SEQ ID NO: 240 - DNA, 20 bp, synthetic construct
  source 1..20: mol_type = other DNA
gcctacttgcccttcttcct

SEQ ID NO: 241 - DNA, 23 bp, synthetic construct
  source 1..23: mol_type = other DNA
agggacctttgcctgtgtgagtc

SEQ ID NO: 242 - DNA, 20 bp, synthetic construct
  source 1..20: mol_type = other DNA
cactcacacagacagaggcc

SEQ ID NO: 243 - DNA, 26 bp, synthetic construct
  source 1..26: mol_type = other DNA
ccaggtgagagtcagggtagtgttca

SEQ ID NO: 244 - DNA, 17 bp, synthetic construct
  source 1..17: mol_type = other DNA
gcttgcgcggcctacgt

SEQ ID NO: 245 - DNA, 21 bp, synthetic construct
  source 1..21: mol_type = other DNA
tcagctctgtgctgaggcgaa

SEQ ID NO: 246 - DNA, 18 bp, synthetic construct
  source 1..18: mol_type = other DNA
gcacaaccctggctgtcc

SEQ ID NO: 247 - DNA, 27 bp, synthetic construct
  source 1..27: mol_type = other DNA
tgaatgcaattgttgttgttaacttgt

SEQ ID NO: 248 - DNA, 20 bp, synthetic construct
  source 1..20: mol_type = other DNA
aagtggttgataaacccacg

SEQ ID NO: 249 - DNA, 20 bp, synthetic construct
  source 1..20: mol_type = other DNA
ccggccagtttctccacctg

SEQ ID NO: 250 - DNA, 20 bp, synthetic construct
  source 1..20: mol_type = other DNA
caggtaaaactgacgcacgg

SEQ ID NO: 251 - DNA, 20 bp, synthetic construct
  source 1..20: mol_type = other DNA
gcttccttacacttcccaag

SEQ ID NO: 252 - DNA, 20 bp, synthetic construct
  source 1..20: mol_type = other DNA
gatcagtgaaacgcaccaga

SEQ ID NO: 253 - DNA, 20 bp, synthetic construct
  source 1..20: mol_type = other DNA
gatggagccagagaggatcc

SEQ ID NO: 254 - DNA, 20 bp, synthetic construct
  source 1..20: mol_type = other DNA
gcagctcaggttctgggaga

SEQ ID NO: 255 - DNA, 20 bp, synthetic construct
  source 1..20: mol_type = other DNA
aagtggttgataaacccacg

SEQ ID NO: 256 - DNA, 20 bp, synthetic construct
  source 1..20: mol_type = other DNA
ccggccagtttctccacctg

SEQ ID NO: 257 - DNA, 20 bp, synthetic construct
  source 1..20: mol_type = other DNA
caggtaaaactgacgcacgg

SEQ ID NO: 258 - DNA, 20 bp, synthetic construct
  source 1..20: mol_type = other DNA
gcttccttacacttcccaag

SEQ ID NO: 259 - DNA, 20 bp, synthetic construct
  source 1..20: mol_type = other DNA
gatcagtgaaacgcaccaga

SEQ ID NO: 260 - DNA, 20 bp, synthetic construct
  source 1..20: mol_type = other DNA
gatggagccagagaggatcc

SEQ ID NO: 261 - DNA, 20 bp, synthetic construct
  source 1..20: mol_type = other DNA
ctactctaattaaacctgta

SEQ ID NO: 262 - DNA, 20 bp, synthetic construct
  source 1..20: mol_type = other DNA
taatgcctcatttaatctat

SEQ ID NO: 263 - DNA, 20 bp, synthetic construct
  source 1..20: mol_type = other DNA
aatgagcaaactctagaatg

SEQ ID NO: 264 - DNA, 20 bp, synthetic construct
  source 1..20: mol_type = other DNA
aatggtccccaggttacatt

SEQ ID NO: 265 - DNA, 20 bp, synthetic construct
  source 1..20: mol_type = other DNA
ctactctaattaaacctgta

SEQ ID NO: 266 - DNA, 20 bp, synthetic construct
  source 1..20: mol_type = other DNA
taatgcctcatttaatctat

SEQ ID NO: 267 - DNA, 20 bp, synthetic construct
  source 1..20: mol_type = other DNA
aatgagcaaactctagaatg

SEQ ID NO: 268 - DNA, 20 bp, synthetic construct
  source 1..20: mol_type = other DNA
aatggtccccaggttacatt

SEQ ID NO: 269 - DNA, 20 bp, synthetic construct
  source 1..20: mol_type = other DNA
tcgacaccaactctagtccg

SEQ ID NO: 270 - DNA, 20 bp, synthetic construct
  source 1..20: mol_type = other DNA
agtcgcttctcgattatggg

SEQ ID NO: 271 - DNA, 20 bp, synthetic construct
  source 1..20: mol_type = other DNA
ccctgggcgttgccctgcag

SEQ ID NO: 272 - DNA, 20 bp, synthetic construct
  source 1..20: mol_type = other DNA
tctcaaatggtataaaactc

SEQ ID NO: 273 - DNA, 20 bp, synthetic construct
  source 1..20: mol_type = other DNA
ggagcttagtcattcacctg

SEQ ID NO: 274 - DNA, 20 bp, synthetic construct
  source 1..20: mol_type = other DNA
atgcctggtagggatgcaaa

SEQ ID NO: 275 - DNA, 20 bp, synthetic construct
  source 1..20: mol_type = other DNA
caccaccaaagtgtagcatc

SEQ ID NO: 276 - DNA, 20 bp, synthetic construct
  source 1..20: mol_type = other DNA
tcgacaccaactctagtccg

SEQ ID NO: 277 - DNA, 20 bp, synthetic construct
  source 1..20: mol_type = other DNA
agtcgcttctcgattatggg

SEQ ID NO: 278 - DNA, 20 bp, synthetic construct
  source 1..20: mol_type = other DNA
ccctgggcgttgccctgcag

SEQ ID NO: 279 - DNA, 20 bp, synthetic construct
  source 1..20: mol_type = other DNA
tctcaaatggtataaaactc

SEQ ID NO: 280 - DNA, 20 bp, synthetic construct
  source 1..20: mol_type = other DNA
ggagcttagtcattcacctg

SEQ ID NO: 281 - DNA, 20 bp, synthetic construct
  source 1..20: mol_type = other DNA
atgcctggtagggatgcaaa

SEQ ID NO: 282 - DNA, 20 bp, synthetic construct
  source 1..20: mol_type = other DNA
caccaccaaagtgtagcatc

SEQ ID NO: 283 - DNA, 20 bp, synthetic construct
  source 1..20: mol_type = other DNA
atgcctggtagggatgcaaa

SEQ ID NO: 284 - DNA, 20 bp, synthetic construct
  source 1..20: mol_type = other DNA
caccaccaaagtgtagcatc

SEQ ID NO: 285 - DNA, 20 bp, synthetic construct
  source 1..20: mol_type = other DNA
atgcctggtagggatgcaaa

SEQ ID NO: 286 - DNA, 20 bp, synthetic construct
  source 1..20: mol_type = other DNA
caccaccaaagtgtagcatc

SEQ ID NO: 287 - DNA, 20 bp, synthetic construct
  source 1..20: mol_type = other DNA
atgcctggtagggatgcaaa

SEQ ID NO: 288 - DNA, 20 bp, synthetic construct
  source 1..20: mol_type = other DNA
caccaccaaagtgtagcatc

SEQ ID NO: 289 - DNA, 27 bp, synthetic construct
  source 1..27: mol_type = other DNA
ggggagcccaagggcacgccctggcac

SEQ ID NO: 290 - DNA, 28 bp, synthetic construct
  source 1..28: mol_type = other DNA
ggcgtgcccttgggctccccgggcgcgt

SEQ ID NO: 291 - DNA, 27 bp, synthetic construct
  source 1..27: mol_type = other DNA
ggggagcccaagggcacgccctggcac

SEQ ID NO: 292 - DNA, 28 bp, synthetic construct
  source 1..28: mol_type = other DNA
ggcgtgcccttgggctccccgggcgcgt

SEQ ID NO: 293 - DNA, 27 bp, synthetic construct
  source 1..27: mol_type = other DNA
ggggagcccaagggcacgccctggcac

SEQ ID NO: 294 - DNA, 28 bp, synthetic construct
  source 1..28: mol_type = other DNA
ggcgtgcccttgggctccccgggcgcgt

SEQ ID NO: 295 - DNA, 27 bp, synthetic construct
  source 1..27: mol_type = other DNA
ggggagcccaagggcacgccctggcac

SEQ ID NO: 296 - DNA, 33 bp, synthetic construct
  source 1..33: mol_type = other DNA
tggggtaacctttgagttctctcagttgggggc

SEQ ID NO: 297 - DNA, 34 bp, synthetic construct
  source 1..34: mol_type = other DNA
actgagagaactcaaaggttaccccagttggggc

SEQ ID NO: 298 - DNA, 33 bp, synthetic construct; identical to SEQ ID NO: 296
  source 1..33: mol_type = other DNA

SEQ ID NO: 299 - DNA, 34 bp, synthetic construct; identical to SEQ ID NO: 297
  source 1..34: mol_type = other DNA

SEQ ID NO: 300 - DNA, 33 bp, synthetic construct; identical to SEQ ID NO: 296
  source 1..33: mol_type = other DNA

SEQ ID NO: 301 - DNA, 34 bp, synthetic construct; identical to SEQ ID NO: 297
  source 1..34: mol_type = other DNA

SEQ ID NO: 302 - DNA, 27 bp, synthetic construct
  source 1..27: mol_type = other DNA
ggggagcccaagggcacgccctggcac

SEQ ID NO: 303 - DNA, 28 bp, synthetic construct
  source 1..28: mol_type = other DNA
ggcgtgcccttgggctccccgggcgcgt

SEQ ID NO: 304 - DNA, 27 bp, synthetic construct
  source 1..27: mol_type = other DNA
ggggagcccaagggcacgccctggcac

SEQ ID NO: 305 - DNA, 28 bp, synthetic construct
  source 1..28: mol_type = other DNA
ggcgtgcccttgggctccccgggcgcgt

SEQ ID NO: 306 - DNA, 33 bp, synthetic construct; identical to SEQ ID NO: 296
  source 1..33: mol_type = other DNA

SEQ ID NO: 307 - DNA, 34 bp, synthetic construct; identical to SEQ ID NO: 297
  source 1..34: mol_type = other DNA

SEQ ID NO: 308 - DNA, 33 bp, synthetic construct; identical to SEQ ID NO: 296
  source 1..33: mol_type = other DNA

SEQ ID NO: 309 - DNA, 34 bp, synthetic construct; identical to SEQ ID NO: 297
  source 1..34: mol_type = other DNA

SEQ ID NO: 310 - DNA, 27 bp, synthetic construct
  source 1..27: mol_type = other DNA
ggggagcccaagggcacgccctggcac

SEQ ID NO: 311 - DNA, 28 bp, synthetic construct
  source 1..28: mol_type = other DNA
ggcgtgcccttgggctccccgggcgcgt

SEQ ID NO: 312 - DNA, 28 bp, synthetic construct
  source 1..28: mol_type = other DNA
ggcgtgcccttgggctccccgggcgcgt

SEQ ID NO: 313 - DNA, 27 bp, synthetic construct
  source 1..27: mol_type = other DNA
ggggagcccaagggcacgccctggcac

SEQ ID NO: 314 - DNA, 28 bp, synthetic construct
  source 1..28: mol_type = other DNA
ggcgtgcccttgggctccccgggcgcgt

SEQ ID NO: 315 - DNA, 27 bp, synthetic construct
  source 1..27: mol_type = other DNA
ggggagcccaagggcacgccctggcac

SEQ ID NO: 316 - DNA, 28 bp, synthetic construct
  source 1..28: mol_type = other DNA
ggcgtgcccttgggctccccgggcgcgt

SEQ ID NO: 317 - DNA, 33 bp, synthetic construct; identical to SEQ ID NO: 296
  source 1..33: mol_type = other DNA

SEQ ID NO: 318 - DNA, 34 bp, synthetic construct; identical to SEQ ID NO: 297
  source 1..34: mol_type = other DNA

SEQ ID NO: 319 - DNA, 34 bp, synthetic construct; identical to SEQ ID NO: 297
  source 1..34: mol_type = other DNA

SEQ ID NO: 320 - DNA, 33 bp, synthetic construct; identical to SEQ ID NO: 296
  source 1..33: mol_type = other DNA

SEQ ID NO: 321 - DNA, 34 bp, synthetic construct; identical to SEQ ID NO: 297
  source 1..34: mol_type = other DNA

SEQ ID NO: 322 - DNA, 33 bp, synthetic construct; identical to SEQ ID NO: 296
  source 1..33: mol_type = other DNA

SEQ ID NO: 323 - DNA, 34 bp, synthetic construct; identical to SEQ ID NO: 297
  source 1..34: mol_type = other DNA

SEQ ID NO: 324 - DNA, 43 bp, synthetic construct
  source 1..43: mol_type = other DNA
taccgtacaccactgagaccgcggtggttgaccagacaaacct

SEQ ID NO: 325 - DNA, 44 bp, synthetic construct
  source 1..44: mol_type = other DNA
gtctggtcaaccaccgcggtctcagtggtgtacggtacaaacct

SEQ ID NO: 326 - DNA, 29 bp, synthetic construct
  source 1..29: mol_type = other DNA
acgacggcggtctccgtcgtcaggatcat

SEQ ID NO: 327 - DNA, 29 bp, synthetic construct
  source 1..29: mol_type = other DNA
acgacggagaccgccgtcgtcgacaagcc

SEQ ID NO: 328 - DNA, 27 bp, synthetic construct
  source 1..27: mol_type = other DNA
cctacatgctcgaagggcgtatgcgcc

SEQ ID NO: 329 - DNA, 26 bp, synthetic construct
  source 1..26: mol_type = other DNA
acgcccttcgagcatgtaggtcacgg

SEQ ID NO: 330 - DNA, 13 bp, synthetic construct
  source 1..13: mol_type = other DNA
gggtttatcaacc

SEQ ID NO: 331 - DNA, 13 bp, synthetic construct
  source 1..13: mol_type = other DNA
gtggagaaactgg

SEQ ID NO: 332 - DNA, 13 bp, synthetic construct
  source 1..13: mol_type = other DNA
tgcgtcagtttta

SEQ ID NO: 333 - DNA, 13 bp, synthetic construct
  source 1..13: mol_type = other DNA
gggaagtgtaagg

SEQ ID NO: 334 - DNA, 13 bp, synthetic construct
  source 1..13: mol_type = other DNA
ggtgcgtttcact

SEQ ID NO: 335 - DNA, 13 bp, synthetic construct
  source 1..13: mol_type = other DNA
tcctctctggctc

SEQ ID NO: 336 - DNA, 13 bp, synthetic construct
  source 1..13: mol_type = other DNA
cccagaacctgag

SEQ ID NO: 337 - DNA, 13 bp, synthetic construct
  source 1..13: mol_type = other DNA
gggtttatcaacc

SEQ ID NO: 338 - DNA, 13 bp, synthetic construct
  source 1..13: mol_type = other DNA
gtggagaaactgg

SEQ ID NO: 339 - DNA, 13 bp, synthetic construct
  source 1..13: mol_type = other DNA
tgcgtcagtttta

SEQ ID NO: 340 - DNA, 13 bp, synthetic construct
  source 1..13: mol_type = other DNA
gggaagtgtaagg

SEQ ID NO: 341 - DNA, 13 bp, synthetic construct
  source 1..13: mol_type = other DNA
ggtgcgtttcact

SEQ ID NO: 342 - DNA, 13 bp, synthetic construct
  source 1..13: mol_type = other DNA
tcctctctggctc

SEQ ID NO: 343 - DNA, 13 bp, synthetic construct
  source 1..13: mol_type = other DNA
aggtttaattaga

SEQ ID NO: 344 - DNA, 13 bp, synthetic construct
  source 1..13: mol_type = other DNA
gattaaatgaggc

SEQ ID NO: 345 - DNA, 13 bp, synthetic construct
  source 1..13: mol_type = other DNA
tctagagtttgct

SEQ ID NO: 346 - DNA, 13 bp, synthetic construct
  source 1..13: mol_type = other DNA
gtaacctggggac

SEQ ID NO: 347 - DNA, 13 bp, synthetic construct
  source 1..13: mol_type = other DNA
aggtttaattaga

SEQ ID NO: 348 - DNA, 13 bp, synthetic construct
  source 1..13: mol_type = other DNA
gattaaatgaggc

SEQ ID NO: 349 - DNA, 13 bp, synthetic construct
  source 1..13: mol_type = other DNA
tctagagtttgct

SEQ ID NO: 350 - DNA, 13 bp, synthetic construct
  source 1..13: mol_type = other DNA
gtaacctggggac

SEQ ID NO: 351 - DNA, 13 bp, synthetic construct
  source 1..13: mol_type = other DNA
actagagttggtg

SEQ ID NO: 352 - DNA, 13 bp, synthetic construct
  source 1..13: mol_type = other DNA
ataatcgagaagc

SEQ ID NO: 353 - DNA, 13 bp, synthetic construct
  source 1..13: mol_type = other DNA
cagggcaacgccc

SEQ ID NO: 354 - DNA, 13 bp, synthetic construct
  source 1..13: mol_type = other DNA
ttttataccattt

SEQ ID NO: 355 - DNA, 13 bp, synthetic construct
  source 1..13: mol_type = other DNA
gtgaatgactaag

SEQ ID NO: 356 - DNA, 13 bp, synthetic construct
  source 1..13: mol_type = other DNA
gcatccctaccag

SEQ ID NO: 357 - DNA, 13 bp, synthetic construct
  source 1..13: mol_type = other DNA
gctacactttggt

SEQ ID NO: 358 - DNA, 13 bp, synthetic construct
  source 1..13: mol_type = other DNA
actagagttggtg

SEQ ID NO: 359 - DNA, 13 bp, synthetic construct
  source 1..13: mol_type = other DNA
ataatcgagaagc

SEQ ID NO: 360 - DNA, 13 bp, synthetic construct
  source 1..13: mol_type = other DNA
cagggcaacgccc

SEQ ID NO: 361 - DNA, 13 bp, synthetic construct
  source 1..13: mol_type = other DNA
ttttataccattt

SEQ ID NO: 362 - DNA, 13 bp, synthetic construct
  source 1..13: mol_type = other DNA
gtgaatgactaag

SEQ ID NO: 363 - DNA, 13 bp, synthetic construct
  source 1..13: mol_type = other DNA
gcatccctaccag

SEQ ID NO: 364 - DNA, 13 bp, synthetic construct
  source 1..13: mol_type = other DNA
gctacactttggt

SEQ ID NO: 365 - DNA, 13 bp, synthetic construct
  source 1..13: mol_type = other DNA
gcatccctaccag

SEQ ID NO: 366 - DNA, 13 bp, synthetic construct
  source 1..13: mol_type = other DNA
gctacactttggt

SEQ ID NO: 367 - DNA, 13 bp, synthetic construct
  source 1..13: mol_type = other DNA
gcatccctaccag

SEQ ID NO: 368 - DNA, 13 bp, synthetic construct
  source 1..13: mol_type = other DNA
gctacactttggt

SEQ ID NO: 369 - DNA, 13 bp, synthetic construct
  source 1..13: mol_type = other DNA
gcatccctaccag

SEQ ID NO: 370 - DNA, 13 bp, synthetic construct
  source 1..13: mol_type = other DNA
gctacactttggt

SEQ ID NO: 371 - DNA, 86 bp, synthetic construct
  source 1..86: mol_type = other DNA
gtttaagagctaagctggaaacagcatagcaagtttaaataaggctagtccgttatcaactcgaaagagtggcaccgagtcggtgc

SEQ ID NO: 372 - DNA, 37 bp, synthetic construct
  source 1..37: mol_type = other DNA
cgcggttctatctagttacgcgttaaaccaactagaa